(19) United States
(12) Patent Application Publication
(10) Pub. No.: US 2026/0076918 A1
(43) Pub. Date: Mar. 19, 2026

(54) HETEROTELECHELIC COMPOUNDS

(71) Applicant: KAWASAKI INSTITUTE OF INDUSTRIAL PROMOTION, Kawasaki-shi, Kanagawa (JP)

(72) Inventors: Joachim VAN GUYSE, Kanagawa (JP); Saed ABBASI, Kanagawa (JP); Kazunori KATAOKA, Kanagawa (JP); Satoshi UCHIDA, Kanagawa (JP)

(73) Assignee: KAWASAKI INSTITUTE OF INDUSTRIAL PROMOTION, Kawasaki-shi, Kanagawa (JP)

(21) Appl. No.: 19/109,632

(22) PCT Filed: Sep. 8, 2023
(86) PCT No.: PCT/JP2023/034382

§ 371 Date: Mar. 7, 2025

(30) Foreign Application Priority Data
Sep. 9, 2022 (JP) 2022-143544

(51) Int. Cl. A61K 9/51; A61K 31/7088; A61K 39/385; A61K 49/00; C08G 69/48

(52) U.S. Cl. CPC A61K 9/5146; A61K 31/7088; A61K 39/385; A61K 49/0002; C08G 69/48

(57) ABSTRACT

The present invention relates to biocompatible polymers suitable for drugs and gene delivery, and producing methods thereof. The present invention also enables the creation of compounds with diverse structures by a concise method.

The present invention relates to polycyclic iminoether compounds having different functional groups in both sides, such as a pentafluorophenyl group at one end and an azide group at the other end, derivatives thereof and producing methods thereof.

The present invention relates to heterotelechelic poly(2-oxazoline) derivative compounds.

### BACKGROUND

Polymeric materials are being developed as medical materials, including biomaterials in direct contact with the living body. To be used as biomaterials, it is required to meet various requirements, including functionality, physical properties, non-toxicity, stealth properties, and biocompatibility. As such polymers, applications such as functional particulates, functionalized surfaces, and functional drug carriers using PEG have been attempted, so far. Patent document 1 discloses polymer compositions for forming surfaces for biosensors applying surface plasmon resonance (SPR), having a polyethylene glycol segment with a mercapto group at one end and a functional group or ligand at the other end. Also, patent document 2 discloses heterotelechelic block copolymers with different functional groups at both ends and polyethylene oxide as the hydrophilic polymer, combined with a hydrophobic polymer, which can be used for materials directly applied to the living body. On the other hand, PEG has disadvantages, such as the fact that anti-PEG antibodies are detected in a certain percentage of subjects with no experience of treatment with PEG drugs and that the polyether structure of PEG is easily oxidized and degraded. Therefore, the development of polymer materials other than PEG is also expected. Then, medical materials based on poly(2-oxazoline) (POx) derivative compounds are expected to be developed as new, more stable polymer materials that maintain the above conditions of stealth property and biocompatibility (Non-Patent Document 1 and Non-Patent Document 2).

### CITATION LIST

### Patent Literature

[Patent Document 1] WO2001/086301
[Patent Document 2] WO1996/033233

### Non-Patent Literature

[Non-Patent Document 1] Macromol.Rapid Commun. 33(19), 2012.
[Non-Patent Document 2] J Mater Sci: Mater Med. 25(5):1211-1225.
[Non-Patent Document 3] Polym. Chem., 2021,12, 6392-6403.

### SUMMARY OF INVENTION

### Problems to be Solved by the Invention

Research has been conducted on poly(2-oxazoline) (POx) derivative compounds (Non-Patent Document 3), but it has yet to control the reaction of both ends of POx to create a wide variety of compounds.

Therefore, the present disclosure provides a technique to control the reaction of both ends of POx to create a wide variety of compounds. Specifically, the inventors provide compounds and derivatives that can freely introduce desired functional groups at each of the two ends of a polymer by utilizing the difference in reactivity of the two ends, as well as their production methods thereof.

The inventors have investigated how to terminate 2-oxazoline polymerisation initiated with pentafluorobenzyl bromide or tosylate as starting materials under different experimental conditions (reaction time, temperature and solvent) and have succeeded in selectively terminating the 2-oxazolinium chain end with various nucleophiles such as N-, O- and S-nucleophiles without reacting para fluoro group. In addition, the inventors have investigated the derivatization of heterotelechelic compounds via para-fluoro substitution and yielding novel heterotelechelic compounds as exemplified in FIG. 2.

As polymeric materials that can be used as biomaterials, the development of new polymeric materials with low immunogenicity and high stability, which can be converted by precisely controlling the functional group to be introduced at the end, is expected. Without limitation, the compounds of the present disclosure may also be useful in the development of such polymeric materials.

### Means for Solving the Problems

The present disclosure provides compounds of formula (I) and producing methods thereof.

As a result of their intensive research, the inventors found that a variety of functional groups can be suitably induced from polymerized poly(2-oxazoline) compounds with a pentafluoro group at one end, depending on the reaction conditions. In other words, the present invention in one aspect, is as follows.

[1]A compound of formula (I)

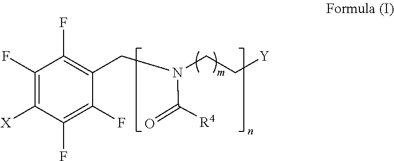

wherein X is halogen, —SR², —OR², —CN, —NHR², —NR²R³, —NHNHR², —N═NR², —N₃, —C≡C—R², —NHOR², —ONR²R³, heteroaryl which may be substituted, a group which can form lipid based nano-particle (e.g., a formulation containing a lipid nano-particle (LNP), liposome, or lipoplex) or micelle including polycationic group, polyanionic group, or lipid soluble groups such as groups derived from fatty acid, preferably F, —SR², —OR², —CN, —NHR², —N₃,

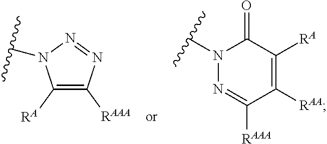

Y is SR², —OR² (provided that when X is F, it is not —OH), —CN, —NHR², —NR²R³, —NHNHR², —N═NR², —N₃, or heteroaryl which may be substituted, preferably —SR², —OR² (provided that when X is F, it is not —OH), —NHR², —NR² R³, —N₃, or

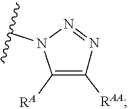

more preferably —SR², —OR² (provided that when X is F, it is not —OH), —NHR², or

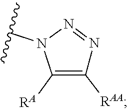

wherein
repeating unit

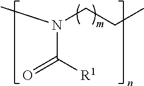

is independent of each occurrence and may be identical or different;
R¹ is selected from the group consisting of alkyl group having 1-40 carbon atoms which may be substituted, alkenyl group having 2-40 carbon atoms which may be substituted, alkynyl group having 2-40 carbon atoms which may be substituted, aryl which may be substituted, heteroaryl which may be substituted;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted, preferably —CH₂CH(OR^{C})CH₂OR^{C},
R^{A}, R^{AA} and R^{AAA} are, independent of each other, selected from the group consisting of hydrogen, alkyl group having 1-20 carbon atoms which may be substituted, alkenyl group having 2-20 carbon atoms which may be substituted, and alkynyl group having 2-20 carbon atoms which may be substituted, or R^{A} and R^{AA} may be linked together to form ring, such as carbocycle, heterocycle, aryl, heterocyclyl, lactone, or lactam, and R^{A}, R^{AA} and/or R^{AAA} may be further linked to functional molecules such as labels, or biofunctional molecules including proteins, nucleic acid, etc.;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, lipid soluble groups such as alkyl group having 1-20 carbon atoms which may be substituted, alkenyl group having 2-20 carbon atoms which may be substituted, and alkynyl group having 2-20 carbon atoms which may be substituted, and oxygen protective groups including acyl groups such as acetyl, ether groups such as methoxymethyl etc.;
said alkyl, alkenyl, and alkynyl is straight chain, branched chain or cyclic chain;
m is integer from 1 to 3;
and
n is integer from 1 to 2000,
or a pharmaceutically acceptable salt thereof.
   [2] The compound according to [1],
wherein X is halogen, —SR², —OR², —NHR², —NR² R³, —NHNHR², —N═NR², —N₃, —NHOR², —ONR²R³, heteroaryl which may be substituted,
Y is SR², —OR², —NHR², —NR² R³, —NHNHR², —N═NR², —N₃, or

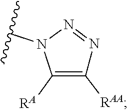

more preferably —SR², —OR², —NHR², or

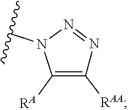

wherein
R¹ is selected from the group consisting of alkyl group having 1-40 carbon atoms which may be substituted, alkenyl group having 2-40 carbon atoms which may be substituted, and alkynyl group having 2-40 carbon atoms which may be substituted, aryl which may be substituted, heteroaryl which may be substituted;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted, preferably —CH₂CH(OR^{C})CH₂OR^{C},
R^{A}, and R^{AA} are, independent of each other, selected from the group consisting of hydrogen, alkyl group having 1-20 carbon atoms which may be substituted, alkenyl group having 2-20 carbon atoms which may be substituted, and alkynyl group having 2-20 carbon atoms which may be substituted, or R^{A} and R^{AA} may be linked together to form carbocycle, heterocycle, aryl, heterocyclyl, lactone, or lactam, and R^{A} and/or R^{AA} may be further linked to labels, or biofunctional molecules including proteins, nucleic acid, etc.;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, lipid soluble groups such as alkyl group having 1-20 carbon atoms which may be substituted, alkenyl group having 2-20 carbon atoms which may be substituted, and alkynyl group having 2-20 carbon atoms which may be substituted, and oxygen protective groups;
said alkyl, alkenyl and alkynyl is straight chain, or branched chain; substituent(s) of alkyl, alkenyl and alkynyl is (are), independent of each other, and also not limited to, but ester group, amino group, azide group, and
n is an integer from 2 to 1000,
or a pharmaceutically acceptable salt thereof.
   [3] The compound according to [1],
wherein X is F, —SR², —OR², —NHR², —N₃, or

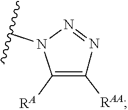

Y is —SR², —OR², —NHR², —N₃, or

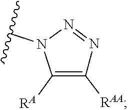

wherein
R¹ is selected from the group consisting of methyl, ethyl, propyl, butyl, —CH₂CH₂COOCH₃, —CH₂CH₂CH₂COOCH₃, —CH₂CH₂CH₂CH₂COOCH₃, butenyl, butynyl, or —CH₂OCH₃;
R² is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives,
R^{A} and R^{AA} are, independent of each other, selected from the group consisting of hydrogen, alkyl group having 1-20 carbon atoms which may be substituted, alkenyl group having 2-20 carbon atoms which may be substituted, and alkynyl group having 2-20 carbon atoms which may be substituted, or R^{A} and R^{AA} may be linked together to form carbocycle, heterocycle, aryl, heterocyclyl, lactone, or lactam, and R^{A} and/or R^{AA} may be further linked to functional molecules such as labels, or biofunctional molecules including proteins, nucleic acid, etc.;
R^{B} is selected from the group consisting of alkyl group having 1-6 carbon atoms which may be substituted;
R^{C} is selected from the group consisting of hydrogen, alkyl group having 1-20 carbon atoms which may be substituted, and alkenyl group having 2-20 carbon atoms which may be substituted;
said alkyl, and alkenyl are straight chain; and
n is an integer from 2 to 2000,
or a pharmaceutically acceptable salt thereof.
   [4] The compound according to [1],
wherein Y is

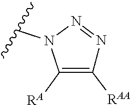

and R^{A} and/or R^{AA} may be linked to functional molecule selecting from the groups consisting of labels, or biofunctional molecules including proteins, and nucleic acid.
   [5] The compound according to [1],
wherein X is —OR² or —SR²,
Y is

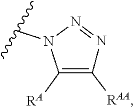

R² is —CH₂CH(OR^{C})CH₂OR^{C}, and
R^{C} is selected from the group consisting of hydrogen, alkyl group having 1-20 carbon atoms which may be substituted, and alkenyl group having 2-20 carbon atoms which may be substituted.
   [6] The compound according to [1],
wherein X is —OR² or —SR²,
R² is —CH₂CH(OR^{C})CH₂OR^{C}, and
R^{C} is selected from the group consisting of alkyl group having 1-20 carbon atoms which may be substituted, and alkenyl group having 2-20 carbon atoms which may be substituted.
   [7] The compound according to [1],
wherein X is —OR² SR², or —NHR²,
R² is —CH₂CH(OR^{C})CH₂OR^{C}, and
R^{C} is H.
   [8] The compound according to [1],
wherein X is —OR², —SR², or —NHR²,
R² is —CH₂CH(OR^{C})CH₂OR^{C}, and
R^{C} is oxygen protective group.
   [9] The compound according to [1],
wherein X is a group which can form lipid based nano-particle (e.g., a formulation containing a lipid nano-particle (LNP), liposome, or lipoplex) or micelle.
   [10] The compound according to [1],
wherein sugar derivative is glucose derivative.
   [11]A composition comprising the compound according to any one of [1]-[10].
   [12]A composition comprising the compound according to any one of [1]-[10],
wherein the composition is vaccine.
   [13]A composition comprising the compound according to any one of [1]-[10],
wherein the composition is used for the diagnostic probes.
   [14]A compound of formula (II)

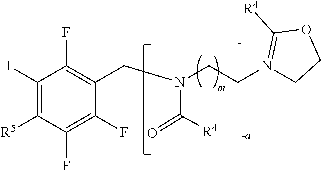

wherein
R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R⁵ is selected from the group consisting of —SR², —OR², —CN, —NHR², —NR² R³, —NHNHR², —N═NR², and —N₃, —NHOR², -and —ONR²R³;
R² and R³ are, independent of each other, selected from the group consisting of hydrogen, alkyl group having 1-6 carbon atoms which may be substituted, alkenyl group having 2-6 carbon atoms which may be substituted, alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case that R⁵ is —NR² R³, R² and R³ may be linked together to form heterocyclyl which may be substituted, preferably —CH₂CH(OR^{C})CH₂OR^{C};
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, alkyl group having 1-20 carbon atoms which may be substituted, or alkenyl group having 2-20 carbon atoms which may be substituted;
said alkyl, and alkenyl are straight chain;
m is integer from 1 to 3;
and
n is integer from 2 to 2000.
   [15] The compound according to [14],
R⁵ is —OR², —SR², or —NHR²,
R² is —CH₂CH(OR^{C})CH₂OR^{C}, and
R^{C} is selected from the group consisting of alkyl group having 1-20 carbon atoms which may be substituted, or alkenyl group having 2-20 carbon atoms which may be substituted.
   [16] The compound according to [14],
R⁵ is —OR², —SR², or —NHR²
R² is —CH₂CH(OR^{C})CH₂OR^{C}, and
R^{C} is H.
   [17] The compound according to [14],
R⁵ is —OR², —SR², or —NHR²
R² is —CH₂CH(OR^{C})CH₂OR^{C}, and
R^{C} is oxygen protective group.
   [18]A method for producing a compound of Formula (III),

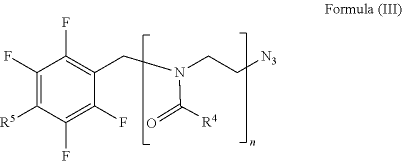

wherein R⁴ is selected from the group consisting of alkyl group having 1-6 carbon atoms which may be substituted, alkenyl group having 2-6 carbon atoms which may be substituted, alkynyl group having 2-6 carbon atoms which may be substituted, aryl which may be substituted, heteroaryl which may be substituted;
R⁵ is selected from the group consisting of halogen, —SR², —OR², —CN, —NHR², —NR² R³, —NHNHR², —N═NR², —N₃, —NHOR², -and —ONR²R³;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, alkyl group having 1-20 carbon atoms which may be substituted, or alkenyl group having 2-20 carbon atoms which may be substituted;
said alkyl, and alkenyl are straight chain; and
n is integer from 2 to 2000,
comprising the following steps;
(a) a compound of Formula (IV)

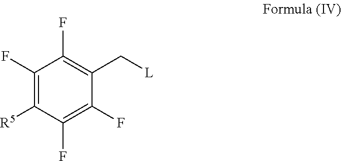

wherein L is a leaving group;
is reacted with a compound of Formula (V)

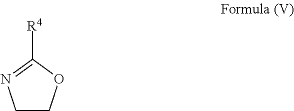

wherein R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
for example, in the presence of base to obtain a compound of Formula (II)

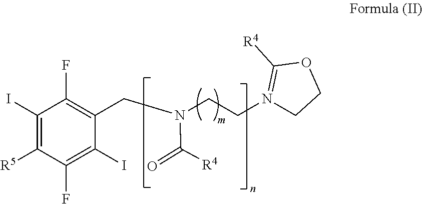

(b) a compound of Formula (II) is reacted with an azide reagent to obtain a compound of Formula (III).
   [19] The method for producing the compound of formula (III) according to [18], wherein the leaving group is selected from the group consisting of halogen, OTf, ON₃, and OTs.
   [20] The method for producing the compound of formula (III) according to [18], wherein the base is selected from the group consisting of Et₃N, DBU, NMP, TBD, KOH, iPr₂NEt, and t-BuOK.
   [21] The method for producing the compound of formula (III) according to [18], wherein the azide reagent is selected from the group consisting of NaN₃, TMSN₃, TsN₃, tetrabutylammonium azide, and diphenyl phosphoryl azide.
   [22]A method for producing the compound of formula (III),

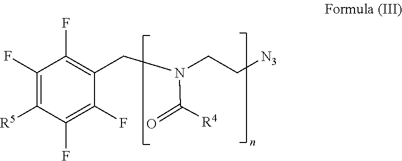

wherein R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R⁵ is selected from the group consisting of halogen, —SR², —OR², —CN, —NHR², —NR² R³, —NHNHR², —N═NR², and —N₃, —NHOR², —ONR²R³;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, alkyl group having 1-20 carbon atoms which may be substituted, or alkenyl group having 2-20 carbon atoms which may be substituted;
said alkyl, and alkenyl are straight chain; and
n is integer from 2 to 2000,
comprising the following steps;
(a) a compound of Formula(IIIa)

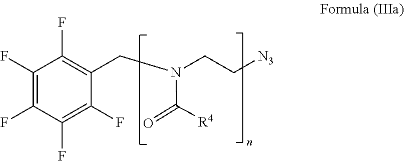

wherein R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
n is integer from 2 to 2000,
is reacted with a nucleophile to obtain a compound of Formula (III)

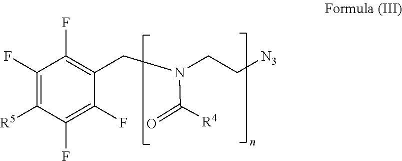

wherein R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R⁵ is selected from the group consisting of halogen, —SR², —OR², —CN, —NHR², —NR² R³, —NHNHR², —N═NR², and —N₃, —NHOR², and —ONR²R³;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted;
R^{C} is selected from the group consisting of hydrogen, alkyl group having 1-20 carbon atoms which may be substituted, or alkenyl group having 2-20 carbon atoms which may be substituted;
said alkyl, and alkenyl are straight chain; and
n is integer from 2 to 2000.
   [23] The method for producing the compound of formula (III) according to [22], wherein the nucleophile is alcohol, phenol, carboxylic acid, amine, azide, thiol, cyan, or alkyne type nucleophile.
   [24] The method for producing the compound of formula (VI),

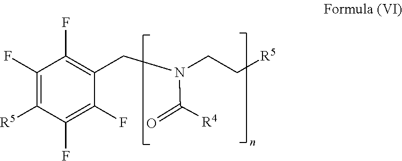

wherein R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R⁵ is selected from the group consisting of halogen, —SR², —OR², —CN, —NHR², —NR² R³, —NHNHR², —N═NR², —N₃, —NHOR², and —ONR²R³;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, alkyl group having 1-20 carbon atoms which may be substituted, or alkenyl group having 2-20 carbon atoms which may be substituted;
said alkyl, and alkenyl are straight chain; and
n is integer from 2 to 100,
in which a compound of formula (IIa)

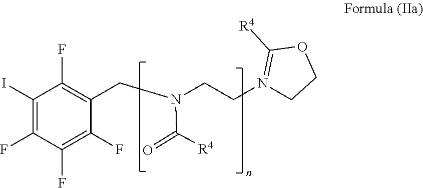

wherein R⁴ is selected from the group consisting of alkyl group having 1-6 carbon atoms which may be substituted, alkenyl group having 2-6 carbon atoms which may be substituted, and alkynyl group having 2-6 carbon atoms which may be substituted;
n is integer from 2 to 2000,
is reacted with nucleophile to obtain the compound of Formula (VI).
   [25]A method for producing the compound of Formula (Ia)

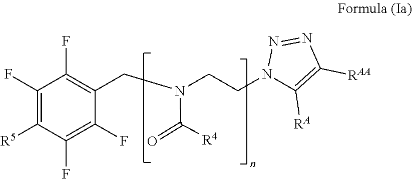

wherein R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R⁵ is selected from the group consisting of —SR², —OR², —CN, —NHR², —NR²R³, —NHNHR², —N═NR², —N₃, —NHOR², and —ONR²R³;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted, preferably —CH₂CH(OR^{C})CH₂OR^{C};
R^{A} and R^{AA} are, independent of each other, selected from the group consisting of hydrogen, alkyl group having 1-20 carbon atoms which may be substituted, alkenyl group having 2-20 carbon atoms which may be substituted, and alkynyl group having 2-20 carbon atoms which may be substituted, or R^{A} and R^{AA} may be linked together to form ring, such as carbocycle, heterocycle, aryl, heterocyclyl, lactone, or lactam, and R^{A} and/or R^{AA} may be further linked to functional molecules such as labels, biofunctional molecules including proteins, nucleic acid, etc.;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, lipid soluble groups such as alkyl group having 1-20 carbon atoms which may be substituted, alkenyl group having 2-20 carbon atoms which may be substituted, and alkynyl group having 2-20 carbon atoms which may be substituted, or oxygen protective groups including acyl groups such as acetyl, ether groups such as methoxymethyl etc.;
said alkyl, alkenyl and alkynyl is straight chain, branched chain or cyclic chain; and
n is integer from 1 to 2000,
wherein the compound of Formula (III)

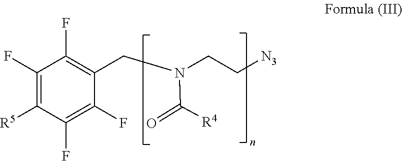

wherein R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R⁵ is selected from the group consisting of halogen, —SR², —OR², —CN, —NHR², —NR² R³, —NHNHR², —N═NR², and —N₃, —NHOR², —ONR²R³;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, alkyl group having 1-20 carbon atoms which may be substituted, or alkenyl group having 2-20 carbon atoms which may be substituted;
said alkyl, and alkenyl are straight chain; and
n is integer from 2 to 2000
is reacted with formula (VII)

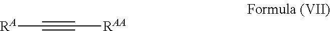

wherein R^{A} and R^{AA} are as shown above,
to obtain the compound of Formula (Ia).
   [26] The method for producing the compound of formula (Ia) according to [25], wherein the reaction carried out with metal catalyst such as Cu catalyst.
   [27] The method for producing the compound of formula (Ia) according to [25], wherein the reaction is carried out under the condition of metal catalyst free.
   [28] The method for producing the compound of formula (Ia) according to [25], wherein the reaction is carried out in water.

### Effect of the Invention

The compounds of the invention are useful as polymeric materials for, drug therapy, gene therapy, laboratory diagnostics, regenerative medicine and pharmaceutical ingredient such as excipients, part of protein conjugate, protein delivery etc., but not limited to.

### BRIEF DESCRIPTION OF THE DRAWINGS

FIG. 1 shows an overview of the pentafluorobenzyl poly(2-substituted-2-oxazoline) compounds in the present invention.

FIG. 2 shows an overview of the heterocyclic benzyl poly(2-substituted-2-oxazoline) compounds in the present invention.

FIG. 3 shows the change over time during the polymerization reaction of ethyl oxazoline with pentafluorobenzyl bromide as starting material.

FIG. 4 shows the spectrum of the polymerization reaction of 2-ethyl propionate oxazoline monomer with pentafluorobenzyl bromide as a starting material and then terminating the reaction with sodium azide.

FIG. 5 shows the ¹⁹FNMR and mass spectrum of the reaction of the para-position fluorine of pentafluorobenzyl poly(2-ethyl-2-oxazoline) azide with sodium azide.

FIG. 6 shows the ¹⁹FNMR and mass spectrum of the reaction of the para-position fluorine of pentafluorobenzyl poly(2-ethyl-2-oxazoline) azide with thioglycerol.

FIG. 7 shows the ¹⁹FNMR and mass spectrum of the reaction of the para-position fluorine of pentafluorobenzyl poly(2-ethyl-2-oxazoline) azide with 3,3-diethoxypropylamine.

FIG. 8 shows the ¹⁹FNMR and mass spectrum of the reaction of the para-position fluorine of pentafluorobenzyl poly(2-ethyl-2-oxazoline) azide with phenol.

FIG. 9 shows the spectrum of the polymerization reaction of 2-ethyloxazoline monomer with pentafluorobenzyl bromide as a starting material and then terminating the reaction with acetic acid.

FIG. 10 shows the change over time during the reaction of 2-ethyl-2-oxazoline with piperidine.

FIG. 11 shows the spectrum of the polymerization reaction of 2-ethyloxazoline monomer with pentafluorobenzyl tosylate as a starting material and then terminating the reaction with sodium azide.

FIG. 12 shows the spectrum of the polymerization reaction of 2-ethyloxazoline monomer with pentafluorobenzyl tosylate as a starting material and then terminating the reaction with 2-Boc-aminoethanethiol.

FIG. 13 shows ¹HNMR of PFP-PEtOx prepared in chlorobenzene and selectively terminated with tert-butyl N-(2-mercaptoethyl) carbamate.

FIG. 14 shows A) a figure showing the shift in retention time of polymers extended by block copolymer synthesis and B) a figure showing the ¹HNMR of PFP-PEtOx prepared in chlorobenzene and selectively terminated with tert-butyl N-(2-mercaptoethyl) carbamate.

FIG. 15 shows the measurement results of C18-PMeOx liposomes.

FIG. 16 shows the molecular weight distributions of C14-PEG, C14-POx and C18-Pox, and firefly luciferase expression in Balb/C mice 4 h after intravenous administration of C14-PEG, C14-POx and C18-POx, respectively.

FIG. 17 shows the results of quantification of firefly luciferase expression in Balb/C mice 4 and 24 h after intravenous administration of C14-PEG, C14-POx and C18-POx respectively, and of quantification of firefly luciferase expression in major organs 24 h after intravenous administration, as assessed by ex-vivo measurements.

FIG. 18 shows the results when mice were immunized with a C14-POxLNP preparation containing mRNA encoding the COVID-19 spike protein.

FIG. 19 shows the ¹HNMR of PFP-PEtOx-N₃.

FIG. 20 shows the ¹⁹FNMR and mass spectrum of the polymerization reaction of 2-ethyloxazoline monomer with pentafluorobenzyl bromide as a starting material and then terminating the reaction with sodium azide.

FIG. 21 shows the ¹⁹FNMR and mass spectrum of the polymerization reaction of 2-methyloxazoline monomer with pentafluorobenzyl bromide as a starting material and then terminating the reaction with sodium azide.

FIG. 22 shows the ¹⁹FNMR and mass spectrum of the polymerization reaction of 2-propyloxazoline monomer with pentafluorobenzyl bromide as a starting material and then terminating the reaction with sodium azide.

FIG. 23 shows the ¹⁹FNMR and mass spectrum of the polymerization reaction of 2-methoxycaeboxyethyl-2-oxazoline monomer with pentafluorobenzyl bromide as a starting material and then terminating the reaction with sodium azide.

FIG. 24 shows the ¹⁹FNMR and mass spectrum of the reaction of the para-position fluorine of pentafluorobenzyl poly(2-ethyl-2-oxazoline) azide with thioglycerol.

FIG. 25 shows the ¹⁹FNMR and mass spectrum of the reaction of the para-position fluorine of pentafluorobenzyl poly(2-ethyl-2-oxazoline) azide with 2-hydroxy ethanethiol.

FIG. 26 shows the ¹⁹FNMR and mass spectrum of the reaction of the para-position fluorine of pentafluorobenzyl poly(2-ethyl-2-oxazoline) azide with 2-amino ethanethiol.

FIG. 27 shows the ¹⁹FNMR and mass spectrum of the reaction of the para-position fluorine of pentafluorobenzyl poly(2-ethyl-2-oxazoline) azide with 3-mercapto propionic acid.

FIG. 28 shows the ¹⁹FNMR and mass spectrum of the reaction of the para-position fluorine of pentafluorobenzyl poly(2-ethyl-2-oxazoline) azide with 2-aminoethane thiol.

FIG. 29 shows the ¹⁹FNMR and mass spectrum of the reaction of the para-position fluorine of pentafluorobenzyl poly(2-ethyl-2-oxazoline) azide with thioglycerol.

FIG. 30 shows the ¹⁹FNMR and mass spectrum of the reaction of the para-position fluorine of pentafluorobenzyl poly(2-ethyl-2-oxazoline) azide with thioglycerol.

FIG. 31 shows the ¹⁹FNMR and mass spectrum of the thioglycoside reaction at the para-position fluorine of pentafluorobenzyl poly(2-ethyl-2-oxazoline) azide.

FIG. 32 shows GPC results for pentafluorobenzyl poly(2-ethyl-2-oxazoline) azide modified with Cysteamine.

FIG. 33 shows the ¹⁹FNMR and mass spectrum of the reaction of the para-position fluorine of pentafluorobenzyl poly(2-methylpropionate-2-oxazoline) azide with 2-aminoethanethiol hydrochloride.

FIG. 34 shows a comparison of para-fluoroamine substitution in DMF when varying equivalents of ethanolamine.

FIG. 35 shows the ¹⁹FNMR and mass spectrum of the reaction of the para-position fluorine of pentafluorobenzyl poly(2-ethyl-2-oxazoline) azide with hydrazine.

FIG. 36 shows the ¹⁹FNMR and mass spectrum of the reaction of the para-position fluorine of pentafluorobenzyl poly(2-ethyl-2-oxazoline) azide with 2-aminoethanol.

FIG. 37 shows the ¹⁹FNMR and mass spectrum of the reaction of the para-position fluorine of pentafluorobenzyl poly(2-ethyl-2-oxazoline) azide with piperidine.

FIG. 38 shows the ¹⁹FNMR and mass spectrum of the reaction of the para-position fluorine of pentafluorobenzyl poly(2-ethyl-2-oxazoline) azide with ethylenediamine.

FIG. 39 shows GPC results for ethylenediamine-modified pentafluorobenzyl poly(2-ethyl-2-oxazoline) azide.

FIG. 40 shows the ¹⁹FNMR and mass spectrum of the reaction of the para-position fluorine of pentafluorobenzyl poly(2-ethyl-2-oxazoline) azide with ethylenediamine.

FIG. 41 shows the ¹⁹FNMR and mass spectrum of the reaction of the para-position fluorine of pentafluorobenzyl poly(2-methyl-2-oxazoline) azide with ethylenediamine.

FIG. 42 shows the ¹HNMR and mass spectrum of p-(3-thiopropane-1,2-diyl difatty acid ester) tetrafluorobenzyl poly(2-methyl-2-oxazoline) azide.

FIG. 43 shows the results of the toxicity tests.

FIG. 44 shows the spectrum of the polymerization reaction of 2-ethyloxazoline monomer with pentafluorobenzyl bromide as a starting material and then terminating the reaction with sodium azide.

FIG. 45 shows the ¹⁹FNMR and mass spectrum of the reaction of the para-position fluorine of pentafluorobenzyl poly(2-ethyl-2-oxazoline) azide with sodium azide.

FIG. 46 shows the ¹⁹FNMR and mass spectrum of the reaction of the para-position fluorine of pentafluorobenzyl poly(2-ethyl-2-oxazoline) azide with thioglycerol.

FIG. 47 shows the ¹⁹FNMR and mass spectrum of the reaction of the para-position fluorine of pentafluorobenzyl poly(2-ethyl-2-oxazoline) azide with thioglycerol.

FIG. 48 shows the ¹⁹FNMR and mass spectrum of the reaction of the para-position fluorine of pentafluorobenzyl poly(2-ethyl-2-oxazoline) azide with various amines.

FIG. 49 shows the mass spectrum of the compound aminated in FIG. 48.

FIG. 50 shows the mass spectrum of the reaction of the para-position fluorine of pentafluorobenzyl poly(2-ethyl-2-oxazoline) azide with phenol.

FIG. 51 shows the ¹⁹FNMR spectrum of pentafluorobenzyl poly(2-ethyl-2-oxazoline) when the reaction was terminated with piperidine.

FIG. 52 shows the ¹⁹FNMR spectrum of pentafluorobenzyl poly(2-ethyl-2-oxazoline) when the reaction was terminated with piperidine.

FIG. 53 shows the ¹⁹FNMR spectrum and mass spectrum of pentafluorobenzyl poly(2-ethyl-2-oxazoline) when the reaction was terminated with piperidine.

FIG. 54 shows the ¹⁹FNMR spectrum of pentafluorobenzyl poly(2-ethyl-2-oxazoline) when the reaction was terminated with 2-(Boc-amino) ethanethiol.

FIG. 55 shows the ¹⁹FNMR spectrum of pentafluorobenzyl poly(2-ethyl-2-oxazoline) when the reaction was terminated with acetic acid.

FIG. 56 shows liposome size and fluorescence intensity of compounds for nanomedicine applications.

FIG. 57 shows firefly luciferase expressions in Balb/C mice injected intravenously with LMPs 4 hours post-injection.

FIG. 58 shows an example application of nanomedicine.

FIG. 59 shows an example application of COVID-19 for vaccines.

FIG. 60 shows the mass spectrum of the reaction of the para-position fluorine of pentafluorobenzyl poly(2-ethyl-2-oxazoline) azide with thioglycerol.

FIG. 61 shows the ¹⁹FNMR spectrum of the reaction of the para-position fluorine of pentafluorobenzyl poly(2-ethyl-2-oxazoline) azide with thioglycerol.

FIG. 62 shows the ¹⁹FNMR spectrum of the reaction of the para-position fluorine of pentafluorobenzyl poly(2-ethyl-2-oxazoline) azide with thioglycerol.

FIG. 63 shows the ¹⁹FNMR spectrum of the reaction of the para-position fluorine of pentafluorobenzyl poly(2-ethyl-2-oxazoline) azide with 2-aminoethanethiol hydrochloride.

FIG. 64 shows ¹⁹FNMR spectrum and mass spectrum of the reaction of the para-position fluorine of pentafluorobenzyl poly(2-ethyl-2-oxazoline) azide with 2,3,4,6-tetra-o-acetyl-1-thioglucose.

FIG. 65 shows the spectrum of the polymerization reaction of 2-methyl propionate-2-oxazoline monomer with pentafluorobenzyl bromide as a starting material and then terminating the reaction with sodium azide.

FIG. 66 shows the ¹HNMR spectrum of the polymerization reaction of 2-methylpropionate-2-oxazoline monomer with pentafluorobenzyl bromide as a starting material and then terminating the reaction with sodium azide.

FIG. 67 shows mass spectrum of the reaction of the para-position fluorine of pentafluorobenzyl poly(2-methylpropionate-2-oxazoline) azide with 2-aminoethanethiol hydrochloride.

FIG. 68 shows mass spectrum of the reaction of the para-position fluorine of pentafluorobenzyl poly(2-methylpropionate-2-oxazoline) azide with 2-aminoethanethiol hydrochloride.

FIG. 68 shows ¹⁹FNMR spectrum of the reaction of the para-position fluorine of pentafluorobenzyl poly(2-methylpropionate-2-oxazoline) azide with 2-aminoethanethiol hydrochloride.

FIG. 70 shows the spectrum of the polymerization reaction of 2-methyloxazoline monomer with pentafluorobenzyl bromide as a starting material and then terminating the reaction with sodium azide.

FIG. 71 shows the spectrum of the polymerization reaction of 2-methyloxazoline monomer with pentafluorobenzyl bromide as a starting material and then terminating the reaction with sodium azide.

FIG. 72 shows NMR spectrum of the reaction of the para-position fluorine of pentafluorobenzyl poly(2-methyl-2-oxazoline) azide with 1-thioglycerol.

FIG. 73 shows the mass spectrum of the reaction of the para-position fluorine of pentafluorobenzyl poly(2-methyl-2-oxazoline) azide with 3-mercapto propionic acid.

FIG. 74 shows the spectrum of the reaction of the para-position fluorine of pentafluorobenzyl poly(2-methyl-2-oxazoline) azide with 3-mercapto propionic acid.

FIG. 75 shows the mass spectrum of the purified PMeOx-lipid conjugate.

FIG. 76 shows the spectrum of the reaction of the para-position fluorine of pentafluorobenzyl poly(2-ethyl-2-oxazoline) azide with ethylene diamine.

FIG. 77 shows the spectrum of the polymerization reaction of 2-propyloxazoline monomer with pentafluorobenzyl bromide as a starting material and then terminating the reaction with sodium azide.

FIG. 78 shows the spectrum of the polymerization reaction of 2-propyloxazoline monomer with pentafluorobenzyl bromide as a starting material and then terminating the reaction with sodium azide.

FIG. 79 shows the spectrum of the polymerization reaction of 2-methoxymethyloxazoline monomer with pentafluorobenzyl bromide as a starting material and then terminating the reaction with sodium azide.

FIG. 80 shows ¹⁹FNMR spectrum of the termination reaction with 2 equivalent of methyl 3-mercaptopropionate in MeCN with various bases.

FIG. 81 shows ¹⁹FNMR spectrum of the termination reaction with 2 equivalents of boc-aminoethanethiol in MeCN with triethylamine.

FIG. 82 shows ¹⁹FNMR and mass spectrum of dioleate-TFP-PEtOx-N₃.

FIG. 83 shows ¹HNMR spectrum of dioleate-TFP-PEtOx-N₃ in dmso-d6.

FIG. 84 shows ¹⁹FNMR and mass spectrum of FIG. 85 shows ¹⁹FNMR and mass spectrum of dioleate-TFP-PMeOx-N₃.

### DESCRIPTION OF EMBODIMENT

In the present specification, halogen is any of F, Cl, Br, or I, preferably F and Cl.

In the present specification, alkyl refers to a linear or branched monovalent hydrocarbon chain comprising only carbon and hydrogen, wherein the carbon-carbon bonds consist of only single bonds, and the hydrocarbon chain is preferably linear.

In the present specification, alkenyl refers to a straight or branched monovalent hydrocarbon chain comprising only carbon and hydrogen and having at least one carbon-carbon double bond in any position. Examples include but are not limited to, vinyl, allyl, 1-propenyl, isopropenyl, butenyl, and decenyl.

In the present specification, alkynyl refers to a straight or branched monovalent hydrocarbon chain comprising only carbon and hydrogen and having at least one carbon-carbon triple bond in any position. Examples include, but are not limited to, ethynyl, propynyl, and butynyl.

In the present specification, carbocyclyl refers to a cyclic structure containing only carbon atoms in the ring, which may be fully or partially saturated. Examples include, but are not limited to, cyclopropyl, cyclopentyl, and cyclohexyl.

In the present specification, heterocyclyl refers to a cyclic structure containing heteroatoms in the ring, which may be fully or partially saturated. Examples include, but are not limited to, pyrrolyl, piperidinyl, morpholinyl, thiophenyl, pyridinyl, and piperazinyl.

In the present specification, substituents in alkyl, alkenyl, alkynyl, carbocyclic, heterocyclic, aryl and heteroaryl include, but are not limited to, halogen, alkyl groups with 1-3 carbon atoms, alkoxy groups with 1-3 carbon atoms, hydroxy groups, ether groups with 1-3 carbon atoms, thioether groups with 1-3 carbon atoms, ester groups with 1-3 carbon atoms in the alkyl group, amino group, amino group which may be substituted by any substituent, for example, polymerization moiety, formyl group, acetal group (such as —CH(OC₂H₅)₂), hemiacetal group, carboxyl group, oxycarbonyl group which may be substituted by any substituent, for example, C₁₋₃ alkyl etc., carbamate which may be substituted by any substituent, for example, C₁₋₃-alkyl, C₁₋₃-haloalkyl etc.(such as —NHCOOtBu), and amide groups which may be substituted by any substituent, for example, C₁₋₃-alkyl, C₁₋₃-haloalkyl et (such as —NHCOCF3).

Oxygen protecting groups herein include, but are not limited to, acetyl groups, methoxymethyl groups, benzyl groups, and benzoyl groups.

Oxygen protecting groups are also well known in the art and include those described in detail in Protecting Groups in Organic Synthesis, T. W. Greene and P. G. M. Wuts, fifth edition, John Wiley & Sons, 2014.

In one aspect of the invention, X is halogen, —SR², —OR², —CN, —NHR², —NR² R³, —NHNHR², —N═NR², —N₃, —C≡C—R², —NHOR², —ONR²R³, heteroaryl which may be substituted, preferably F, —SR², —OR², —NHR², —NR² R³, —NHNHR², —N₃.

In one aspect of the invention, the substituent of heteroaryl which may be substituted include, but are not limited to, for example, halogen, methyl, ethyl, methoxy, methylamino, dimethylamino, carboxy, etc.

In one aspect of the invention, Y is SR², —OR² (provided that when X is F, it is not —OH), —CN, —NHR², —NR² R³, —NHNHR², —N═NR², —N₃, or heteroaryl which may be substituted, preferably —SR², —OR² (provided that when X is F, it is not —OH), —NHR², —NR² R³, —NHNHR², —N₃, or

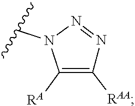

more preferably —SR², —OR² (provided that when X is F, it is not —OH), —NHR², —NR² R³, —N₃, or

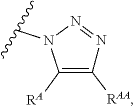

and most preferably —SR², —OR² (provided that when X is F, it is not —OH), —NHR², —NR² R³, —N₃.

In one aspect of the invention, Y is not OH. In one preferable aspect of the invention, when X is F, Y is not —OH.

In one aspect of the invention, R¹ is selected from the group consisting of alkyl group having 1-40 carbon atoms, preferably 1-20 carbon atoms, more preferably 1-18 carbon atoms, which may be substituted, alkenyl group having 2-40 carbon atoms, preferably 2-20 carbon atoms, more preferably 2-18 carbon atoms, which may be substituted, alkynyl group having 2-40 carbon atoms, preferably 2-20 carbon atoms, more preferably 2-18 carbon atoms, which may be substituted, aryl which may be substituted, heteroaryl which may be substituted.

In one aspect of the invention, R¹ is selected from the group consisting of methyl, ethyl, propyl, butyl, pentyl, —CH₂COOCH₃, —CH₂CH₂COOCH₃, —CH₂CH₂CH₂COOCH₃, alcohols and the acetates thereof, such as —CH₂OH, —CH₂CH₂OH, —CH₂OCOCH₃, —CH₂CH₂OCOCH₃.

In one aspect of the invention, R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted, preferably —CH₂CH(OR^{C})CH₂OR^{C}, preferably alkyl group having 1-6 carbon atoms which may be substituted, aryl which may be substituted, —C(═O)R^{B}, sugar derivative, and repeating unit (moiety having the polymerized structure), or in the case of —NR² R³, R² and R³ may be linked together to form heterocyclyl which may be substituted.

In one aspect of the invention, R^{A}, R^{AA} and R^{AAA} are, independent of each other, selected from the group consisting of hydrogen, alkyl group having 1-20 carbon atoms which may be substituted, alkenyl group having 2-20 carbon atoms which may be substituted, and alkynyl group having 2-20 carbon atoms which may be substituted, or R^{A} and R^{AA} may be linked together to form ring, such as carbocycle, heterocycle, aryl, heterocyclyl, lactone, or lactam, and R^{A}, R^{AA} and/or R^{AAA} may be further linked to functional molecules such as labels, or biofunctional molecules including proteins, nucleic acid, etc.;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, preferably alkyl group having 1-6 carbon atoms which may be substituted, alkenyl group having 2-6 carbon atoms which may be substituted, and more preferably alkyl group having 1-6 carbon atoms which may be substituted;
R^{C} is selected from the group consisting of hydrogen, lipid soluble groups such as alkyl group having 1-20 carbon atoms which may be substituted, alkenyl group having 2-20 carbon atoms which may be substituted, and alkynyl group having 2-20 carbon atoms which may be substituted, and oxygen protective groups including acyl groups such as acetyl, ether groups such as methoxymethyl etc., preferably hydrogen, alkyl group having 1-20 carbon atoms which may be substituted, alkenyl group having 2-20 carbon atoms which may be substituted.

In one aspect of the invention, R⁴ is methyl, ethyl, propyl, butyl, pentyl, —CH₂COOCH₃, —CH₂CH₂COOCH₃, —CH₂CH₂CH₂COOCH₃, alcohols and the acetates thereof, such as —CH₂OH, —CH₂CH₂OH, —CH₂OCOCH₃, —CH₂CH₂OCOCH₃, preferably methyl, ethyl, propyl, —CH₂CH₂COOCH₃.

In one aspect of the invention,

In one aspect of the invention, each repeating unit includes, but is not limited to, following,

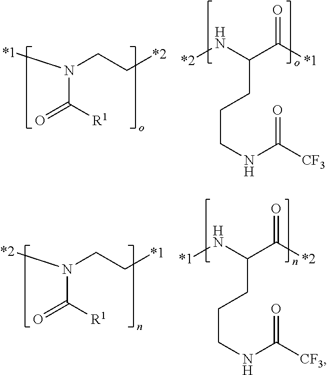

etc.
wherein o is 1 to 2000, preferably, n is 30 to 300; and
1 is ω-end side, and *2 is α-end side in the compound of the present invention.

Polycationic groups herein include, but are not limited to, quaternary ammonium salts, polyamines such as polylysine and polyornithine etc.

Polyanionic groups herein include, but are not limited to, polyaspartate, polyglutamate etc.

Biofunctional molecules herein include, but are not limited to, functional molecules encompassing proteins, nucleic acids, etc.

Functional molecules herein include, but are not limited to, labels, antibodies, antibody fragments, peptides, nucleic acids, aptamers, lipid, carbohydrate etc.

Lipid based nano-particle herein include, but are not limited to, formulations containing a lipid nano-particle (LNP), liposome, or lipoplex etc.

In copolymers herein, each repeating unit is independent of each occurrence and may be identical or different. Copolymers herein include, but are not limited to, block copolymers, statistical copolymer, etc.

Labels herein include, for example, detectable portions or markers for detection based on fluorescence emission, fluorescence polarisation, fluorescence lifetime, fluorescence wavelength, absorbance wavelength, absorbance, Raman, Stokes shift, light scattering, molecular weight, redox, magnetism, high frequency, enzyme reaction, or combinations thereof, but are not limited to them. The labels may also be, for example, fluorophores, chromophores, enzymes, redox labels, radioactive labels, Raman tags, mass tags, isotope elements, magnetic particles, microparticles, and nanoparticles.

In certain embodiments, the labels are preferably fluorophores. The labels used in the present invention may also be used in vivo or in vitro.

In the present specification, PFP denotes pentafluorobenzyl, and PEtOx denotes poly(2-ethyl-2-oxazoline). Thus, for example, “PFP-PEtOx45-N3” is pentafluorobenzyl poly(2-ethyl-2-oxazoline) azide, which is a compound wherein 45 units of (2-ethyl-2-oxazoline) are polymerized, and “PFP-PMeOx50-N3” is pentafluorobenzyl poly (2 (2-methyl-2-oxazoline) azide, which is a compound wherein 50 units of (2-methyl-2-oxazoline) are polymerized.

In the present specification, “α-end” refers to the left-hand end of FIG. 2, i.e. the Y moiety linked to the PFP, and “ω-end” refers to the right-hand end of FIG. 2, i.e. the X moiety.

Leaving groups herein are groups that are dissociated during the reaction, including but not limited to, halogens, OTf, ONs and OTs.

In the polymer compounds described in the specification, the number indicating their polymerization degree does not indicate only the exact number of polymerization degree, but within the range with errors of 10%.

In one aspect of the invention, the compound of Formula (I) includes, for example, following compounds;

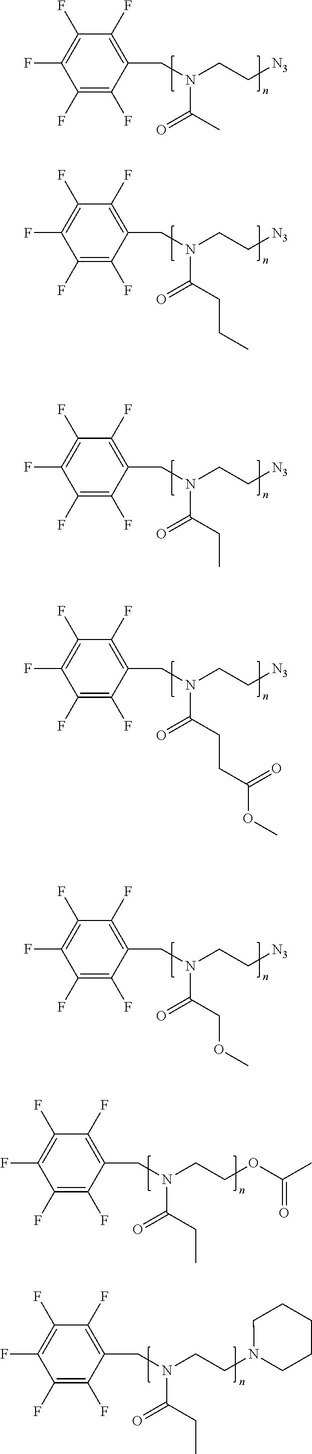

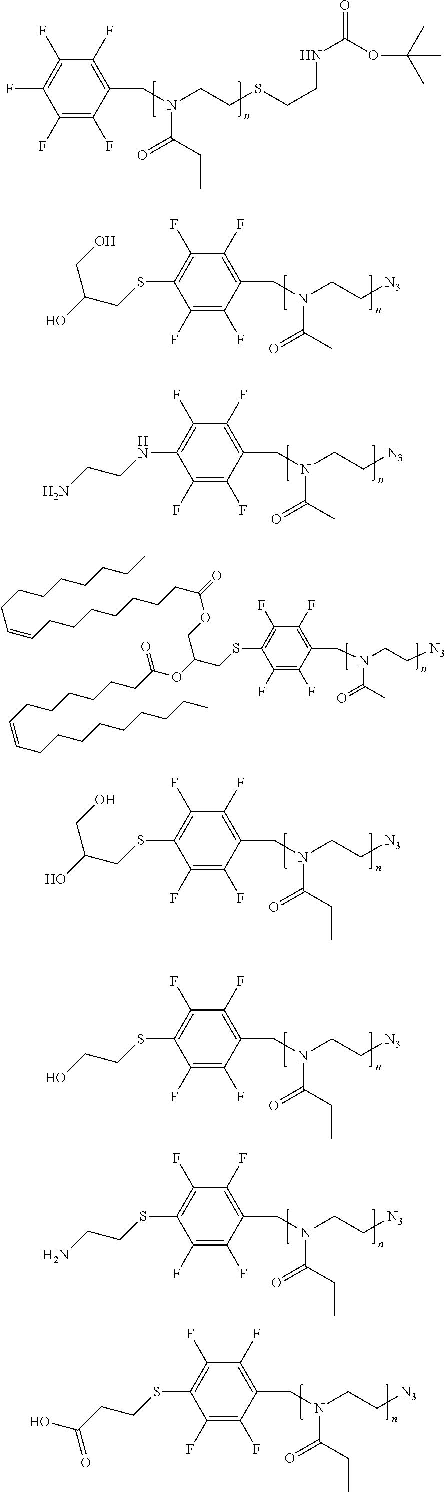

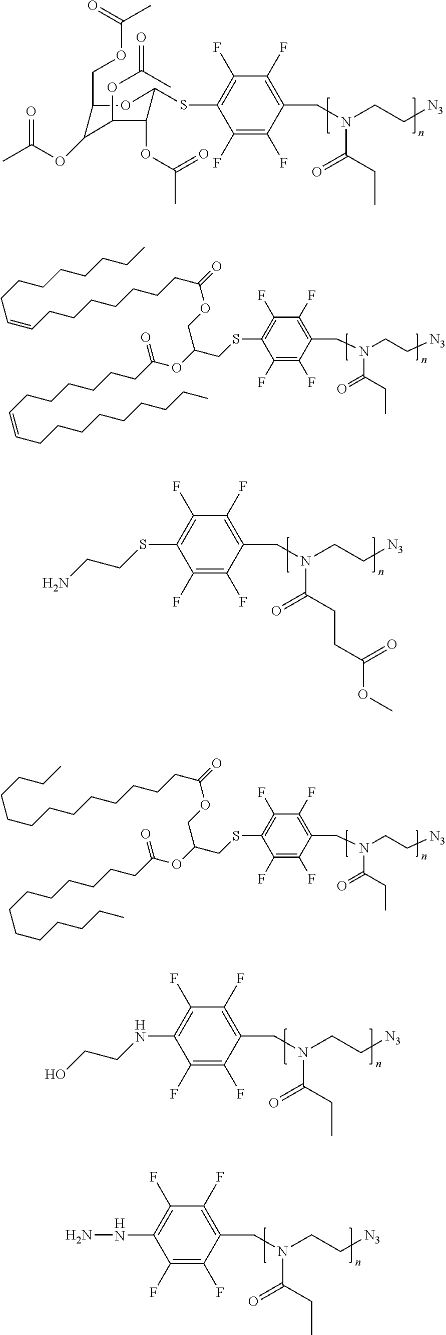

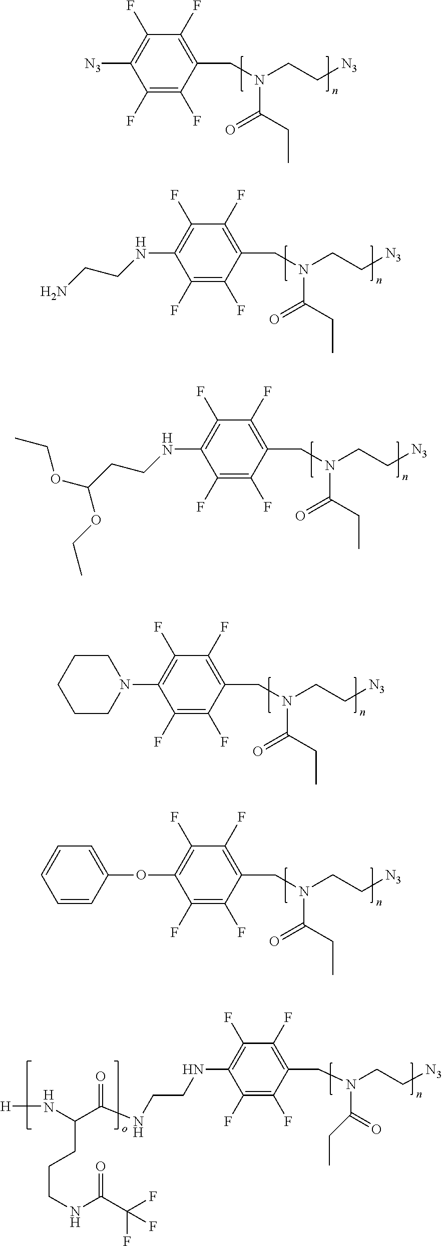

wherein n is 1 to 2000, preferably, n is 2 to 1000, more preferably 3 to 500, and most preferably 30 to 150;
o is 1 to 2000, preferably, n is 1 to 2000, more preferably 3 to 500, and most preferably 30 to 150.

In one aspect of the invention, the compound of Formula (I) includes, for example, following compounds;

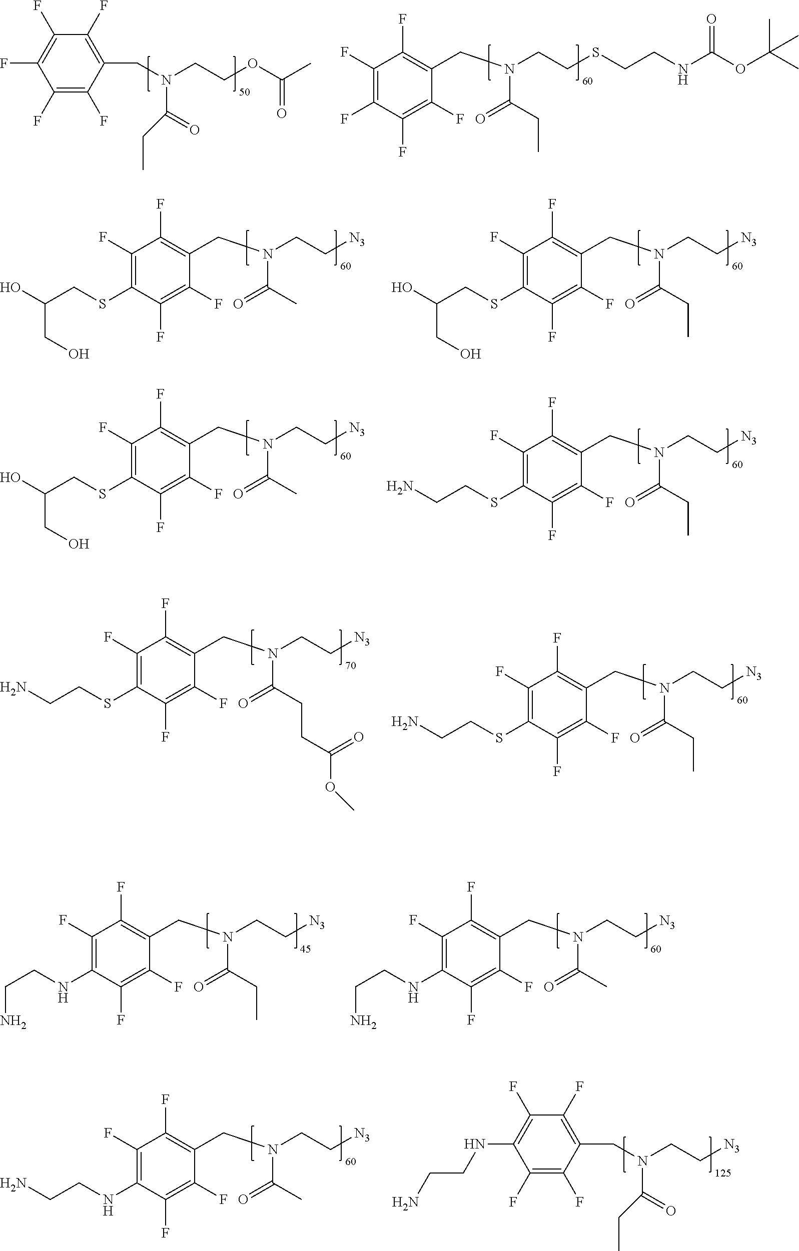

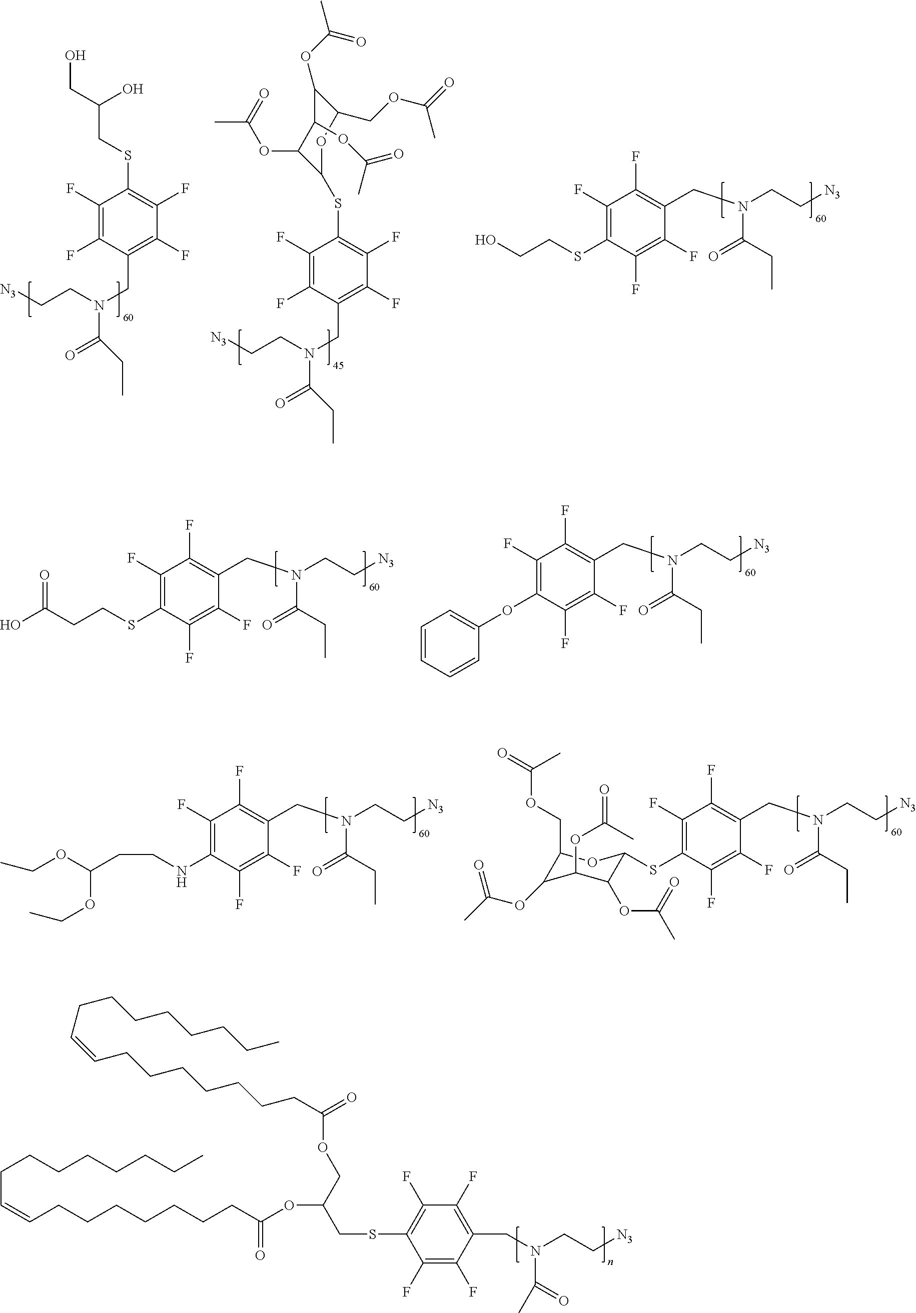

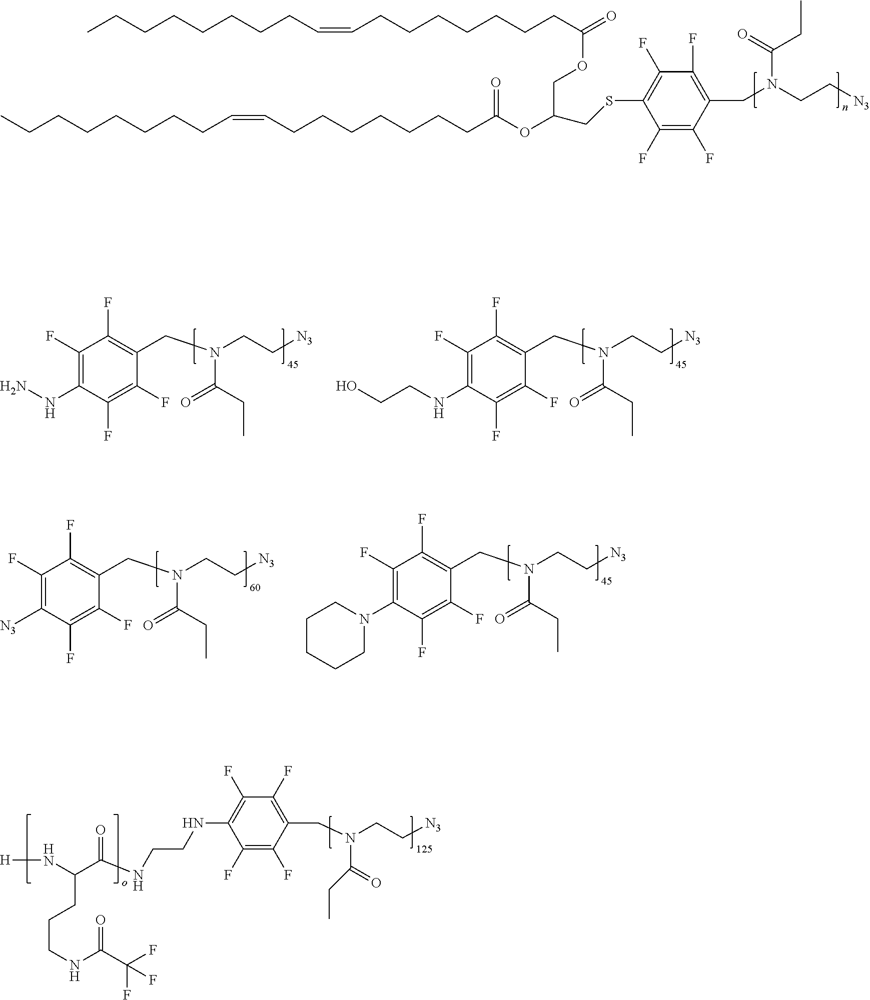

wherein o is 50 to 200.

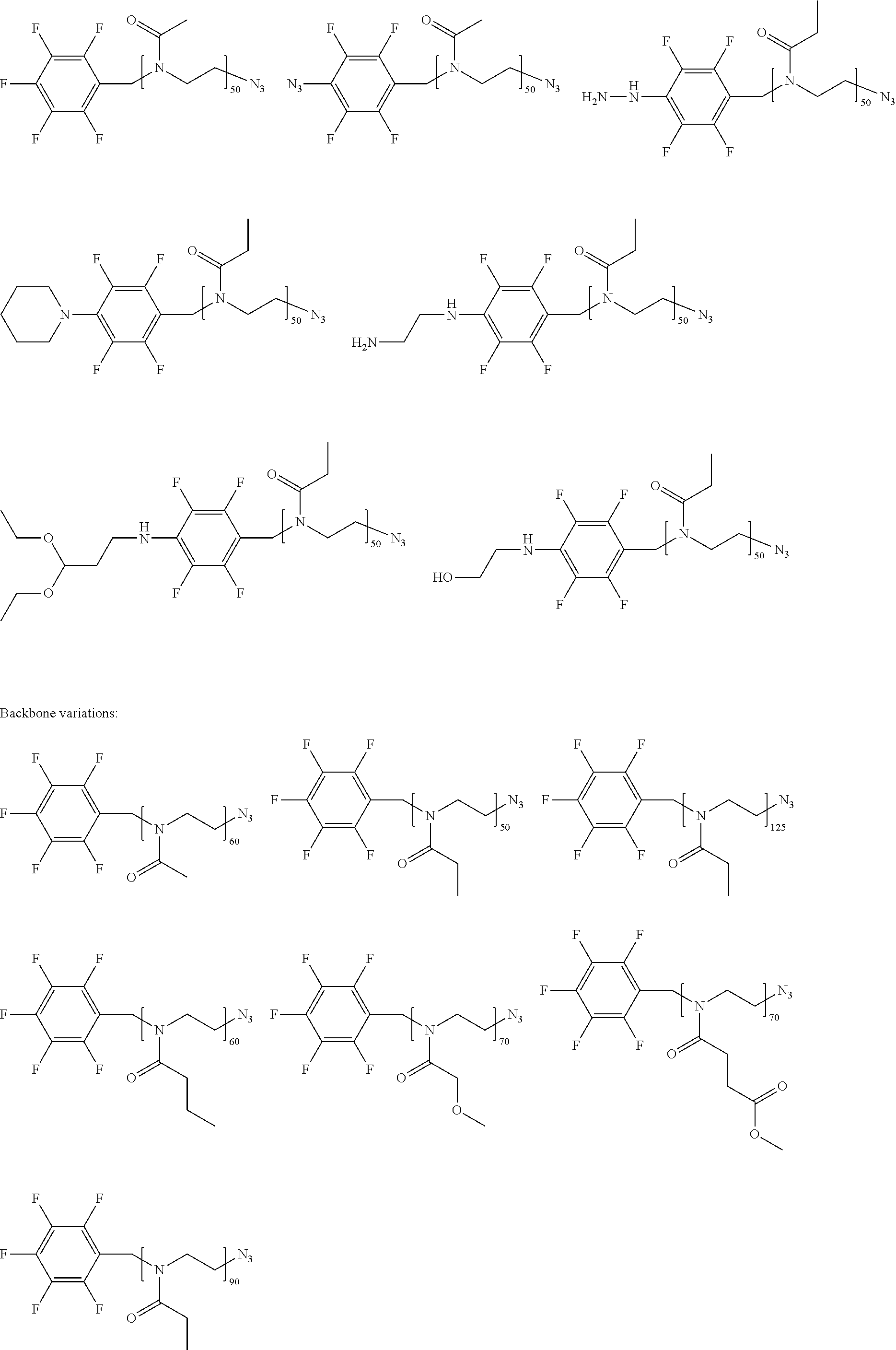

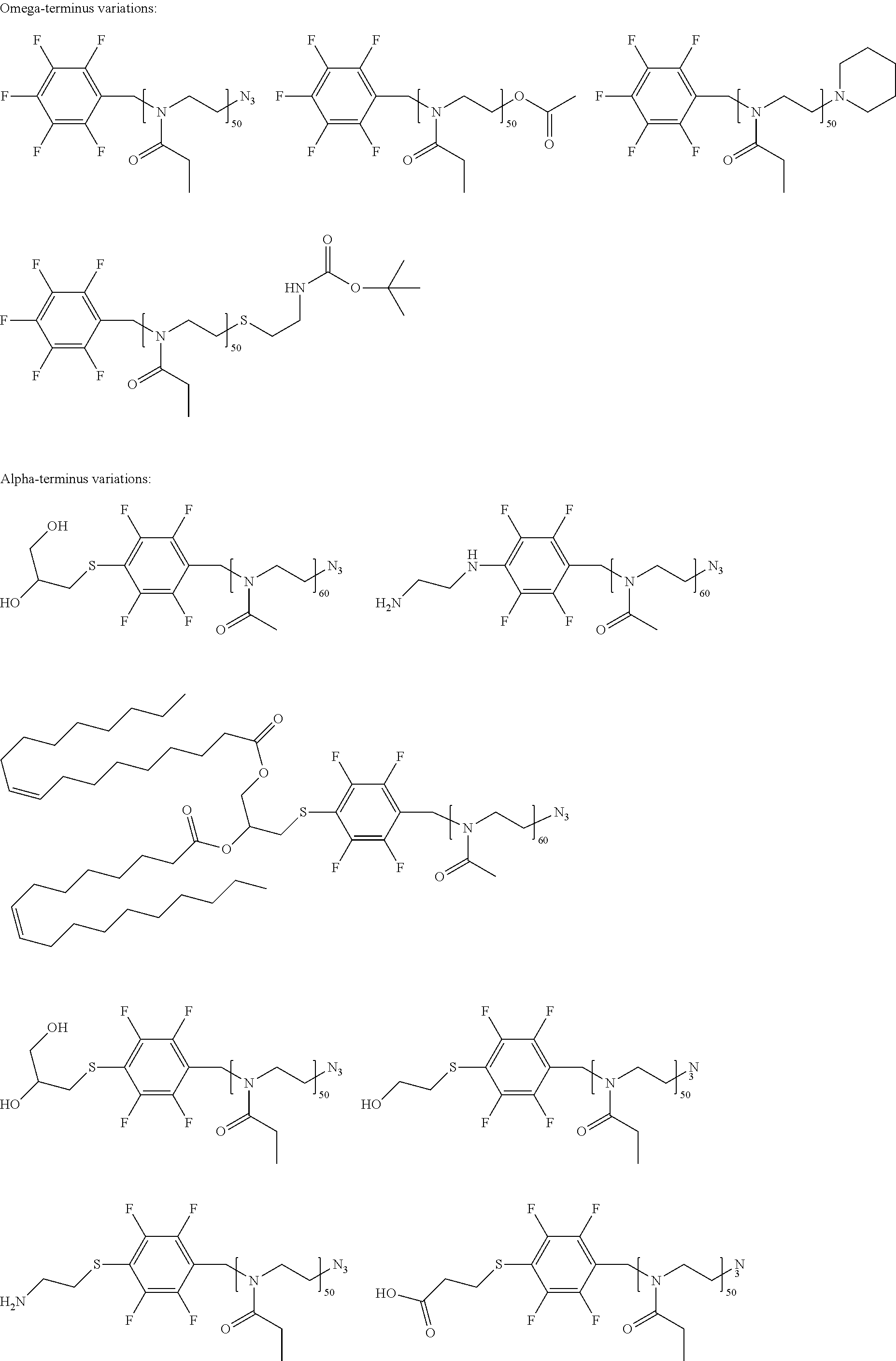

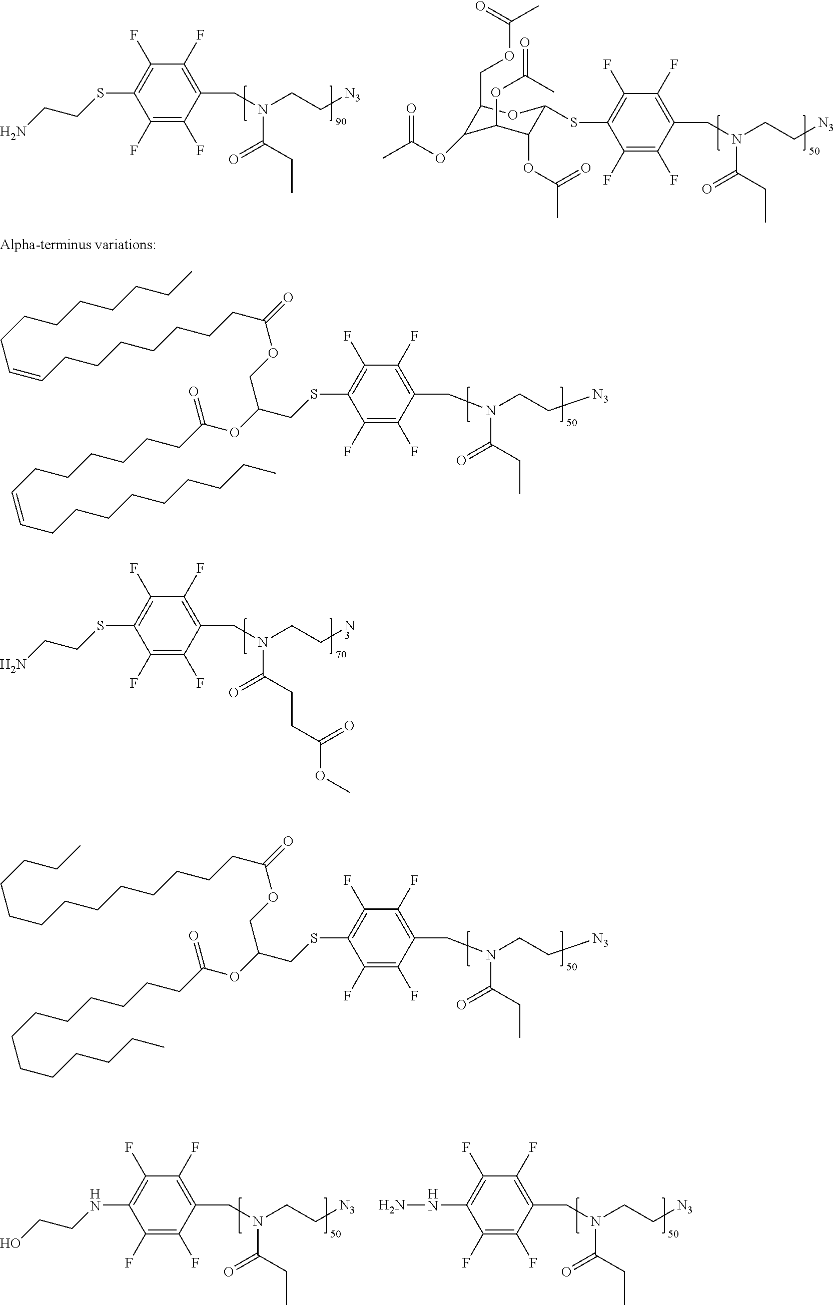

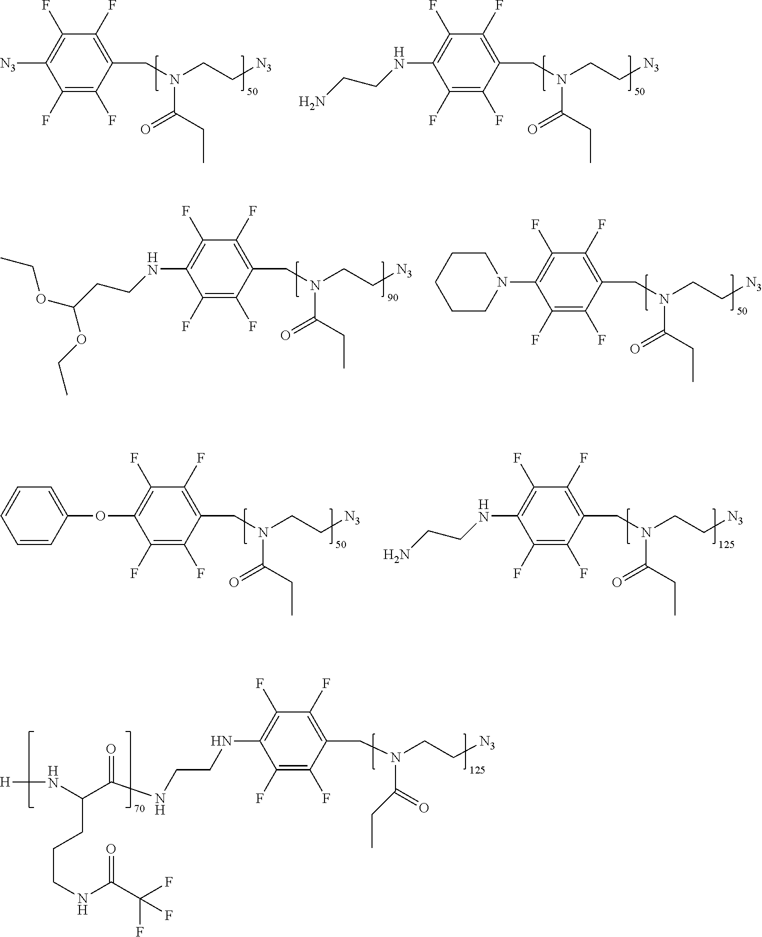

or a pharmaceutically acceptable salt thereof.

In one aspect of the invention, the compound of Formula (IIIa) includes, for example, following compounds;

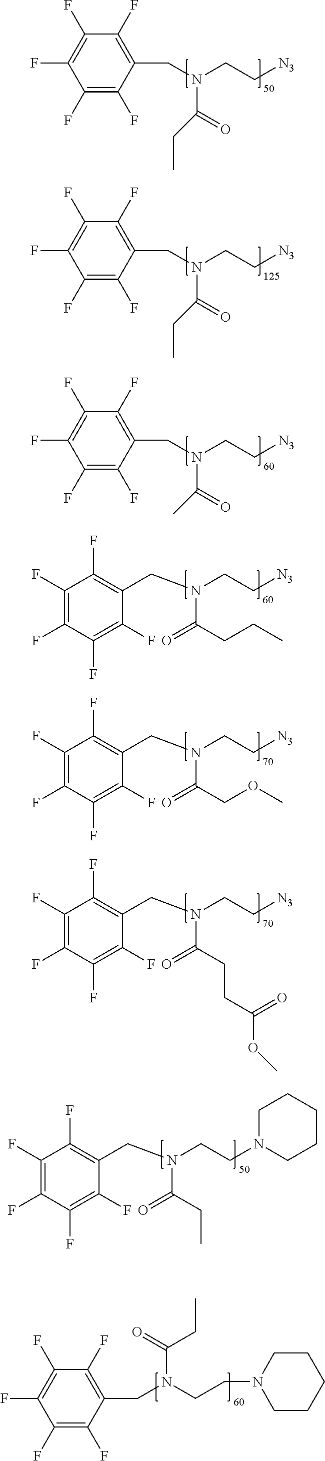

In one aspect of the invention, the copolymer includes, for example,

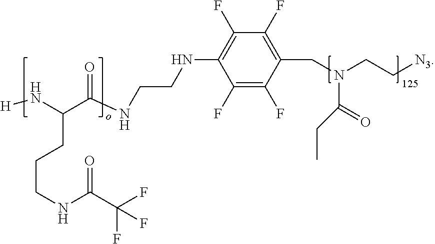

wherein o is 50 to 200.

Preferred nucleophiles used in the 2-oxazoline polymerisation of the present invention during the polymerisation terminating reaction at ω-end include, but are not limited to, azidising reagents, thiols, amines, and carboxylic acids, and salt thereof, such as NaN₃. The solvents used during this termination reaction at ω-end are preferably, but not limited to, chlorobenzene, acetonitrile, dimethylacetamide, CH₂Cl₂, N-methylpyrrolidone (NMP), sulfolane, nitrobenzene, benzonitrile, and ethyl acetate. The termination reaction at ω-end may carry out under neat conditions. The reaction temperature in the termination reaction at ω-end is from −70 to 140° C., preferably from 0 to 50° C., more preferably from 0 to 30° C. Bases can be used in the termination reaction at the a-end, including triethylamine (TEA), diisopropylethylamine (DIPEA), Na₂CO₃, and DBU, etc., but not limited to.

Preferred nucleophiles used in the fluorine substitution reaction at α-end of the present invention include, but are not limited to, azidation reagents, thiols, amines, carboxylic acids, alcohols, phenol, hydrazines, hydrazides, thio-carboxylic acids, etc., such as NaN₃, and phenol. Solvents used during this α-end reaction preferably include, but are not limited to, DMF, NMP, acetonitrile (ACN), water, alcohols, DMSO, and sulfane. The reaction temperature in the a-end fluorine substitution reaction is from 0 to 100° C., preferably from 20 to 80° C., more preferably from 20 to 70° C. Any bases can also be used in the a-end fluorine substitution reaction, including but not limited to triethylamine (TEA), diisopropylethylamine (DIPEA), potassium t-butoxide (KOtBu), diazabicycloundecene (DBU), pyrimid[1,2-a]pyrimidine (TBD), potassium hydroxide (KOH), sodium carbonate (Na₂CO₃), and potassium carbonate (Na₂CO₃).

In the present invention, all of the reactions described herein can be performed under suitable conditions. In the present invention, from the viewpoint of selectively carrying out the polymerization terminating reaction at the a-end and the fluorine substitution. When a base is used, it is preferable to be a weak base, preferably triethylamine, diisopropylethylamine and more preferably no base.

In order to make the reaction selective, the appropriate combination of solvent, temperature and base should be selected during the polymerisation terminating reaction at the wend.

### Selective Termination

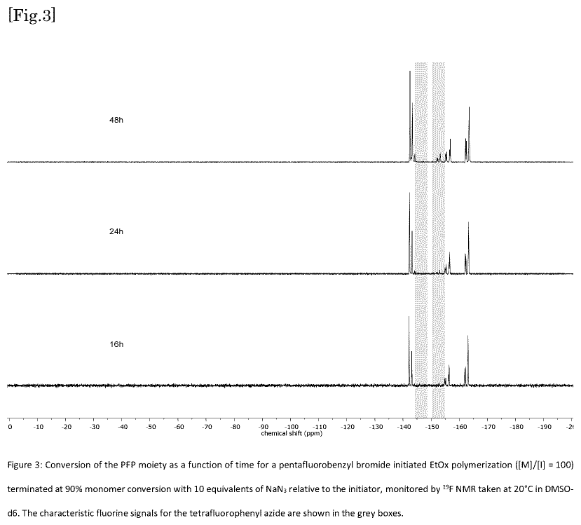

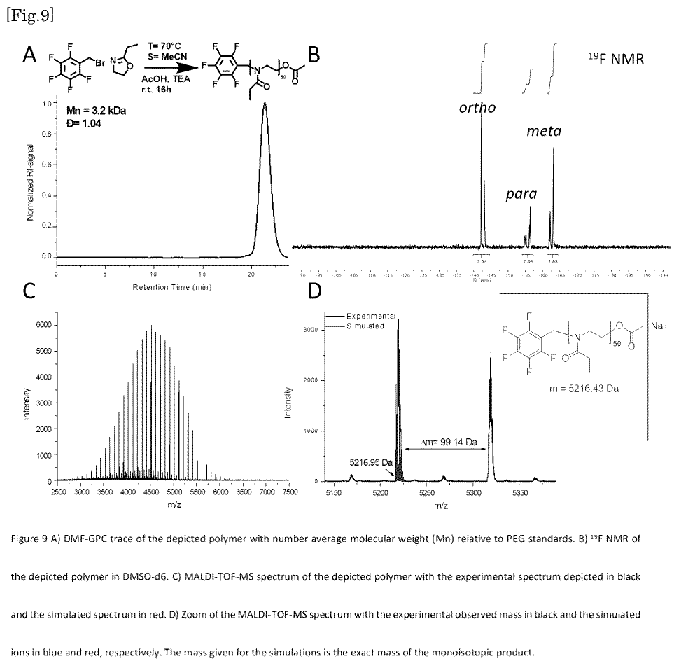

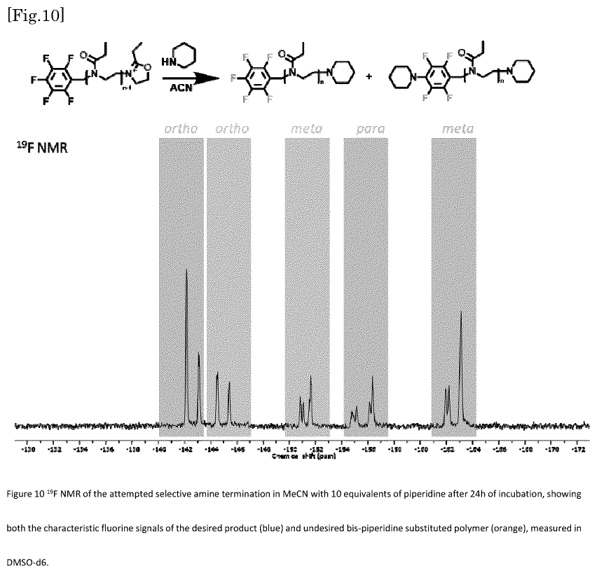

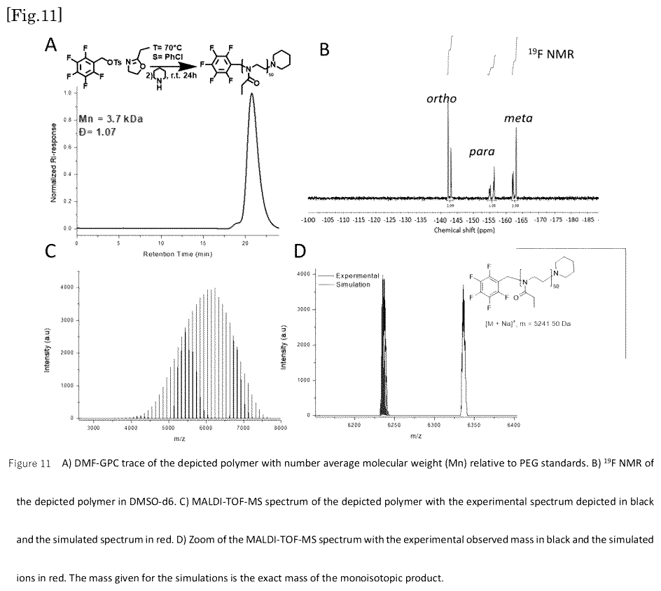

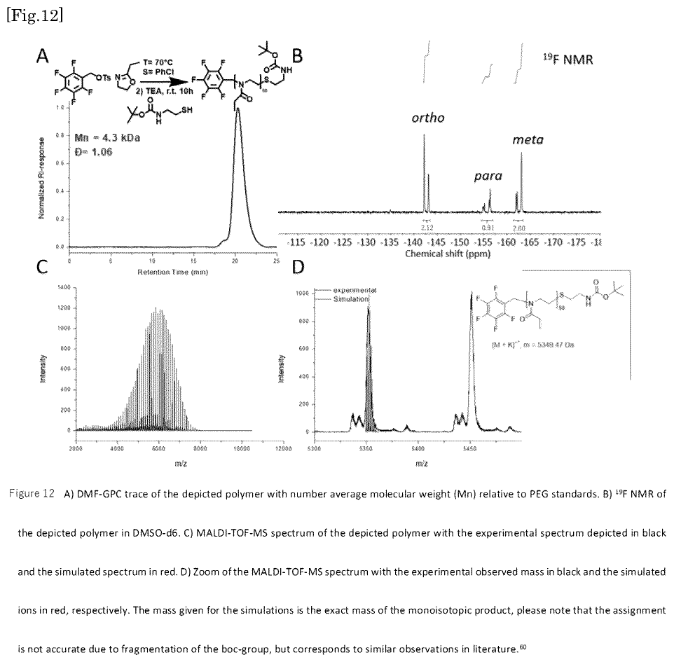

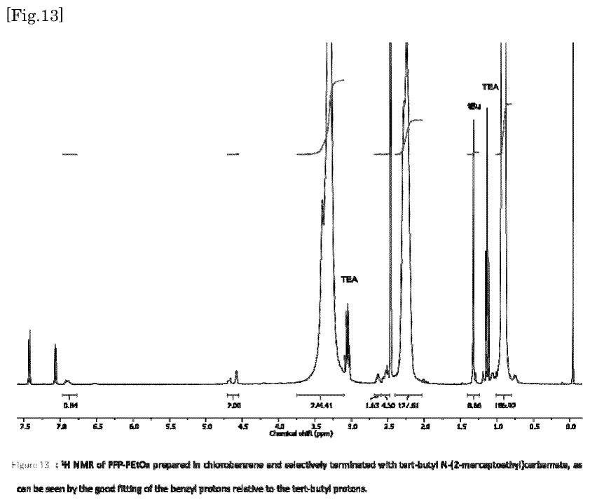

The termination step of the CROP of 2-oxazolines typically requires the addition of excess of the N-, O- or S-nucleophiles to ensure quantitative reaction, and avoid undesired side-reactions, such as overalkylation in the case of amines. Hence we investigated the termination of pentafluorobenzyl bromide/tosylate initiated 2-oxazoline polymerizations under different experimental conditions (reaction time, temperature and solvent), to enable selective termination of the living 2-oxazolinium chain end with different N-, O-, and S-nucleophiles, consequently affording heterotelechelic POx bearing the PFP-moiety at the a-end, and azide, amine, ester or thioether groups at the ω-end. As can be seen in FIG. 3 (in patent), the reaction of azide anion with the 2-oxazolinium requires careful control of reaction time in order to provide selectivity when performing the termination reaction at room temperature. Under these conditions acetate anions could also be utilized as nucleophiles to afford the desired heterotelechelics (FIG. 9). On the other hand, when amines (FIG. 10) or aliphatic thiols (FIG. XX2-3) were applied as nucleophiles to react with the 2-oxazolinium in the presence of the PFP moiety, no selectivity was observed under the applied conditions. Utilizing piperidine as a model amine, a mixture of products was obtained when performing the reaction in acetonitrile. When thiols were applied as the nucleophile and ACN as the solvent, a screening revealed that a similar situation occurred even with the application of mild bases to deprotonate the thiol. Even careful optimization of the reaction temperature and reaction time proved that in these situations, selective reaction could not be achieved in polar solvents, such as ACN. In order to facilitate selective termination of the 2-oxazolinium in the presence of the PFP moiety, we investigated the use of chlorobenzene as a solvent, in order to energetically favor the reaction with the 2-oxazolinium. Under these conditions and the applied reaction times, selective termination was afforded for amines and aliphatic thiols as seen in FIG. 11-13.

Preferred combination of reaction conditions includes,
Acetonitrile, 70° C., no base
NMP, 20° C., no base
Chlorobenzene, room temperature, no base
Chlorobenzene, 70° C., no base
Chlorobenzene, room temperature, no base,
Acetonitrile, 20° C., no base,
Acetonitrile, 20° C., base,
Chlorobenzene, 20° C., base,
   but not limited to.

Preferred embodiments of the invention are described below, but these are provided only for the understanding of the present disclosure and the scope of the present disclosure is not to be understood as limited to the following description.

### EXAMPLES

### Materials and Methods

All chemicals were used as received unless otherwise specified. Pentafluorobenzyl bromide (TCI, 99%), pentafluorobenzyl tosylate (TCI >98%), chlorobenzene (Wako 99%), acetonitrile (Merck, DNA-synthesis grade), 2-ethyl-2-oxazoline (TCI >98%), 2-methyl-2-oxazoline (TCI, >98%), 2-propyl-2-oxazoline (TCI >98%), CaH₂ (Sigma Aldrich), NaOH (Wako), piperidine (Sigma-Aldrich 99%, redistilled), ethylenediamine (TCI), aminoethanol (TCI), phenol (sigma, ≥99), 1-thio-β-D-glucose tetraacetate (Cayman Chemical), 1,8-diazabicyclo(5.4.0)undec-7-ene (DBU), potassium tert-butoxide, triethylamine(TCI), tosyl isocyanate (sigma), dimethylformamide (DMF), diethyl ether (nacalai), N-methylpyrrolidone (NMP) (Wako, super dry), sulfuric acid (Wako, 98%), magnesium sulfate (Sigma-Aldrich), dinitrofluorobenzene(TCI), cysteamine hydrochloride (TCI), triazabicyclodecene (TBD, 98%, TCI), BaO (90%, Acros Organics), ninhydrin (sigma, ACS reagent), sodium carbonate (Sigma Aldrich), ethyl acetate (Nacalai), sodium azide (≥99.5%, sigma). Deuterated solvents were purchased from Cambridge isotope Laboratories. CleanCap® 5moU FLuc mRNA (Trilink), DSPC and D-Lin-MC3-DMA (MedChemExpress), cholesterol (Sigma), DMG-PEG2000 (Avanti), DiD (Invitrogen), DOPC (NOF).

Sodium azide was dried at 180° C. before use. Sodium carbonate was ground with pestle and mortar and subsequently dried at 180° C. 2-methoxycarbonylethyl-2-oxazoline (C2MestOx) was synthesized following a previously reported protocol and was further purified by fractional distillation over BaO and ninhydrin and isolated as a white crystalline solid. Pentafluorobenzyl bromide was distilled in vacuo and stored at −30° C.

Acetonitrile purification deviated from literature, as we found that distillation over CaH₂ or acetonitrile obtained from solvent purification systems still contained amine impurities, which was confirmed by Sanger's reagent. Therefore DNA-synthesis grade acetonitrile was transferred to a distillation setup equipped with a fractional distillation column (40 cm), and the solution was refluxed for 2 hours over tosyl isocyanate (1 mL/100 ml of acetonitrile) under inert atmosphere to remove any nucleophilic impurities. Next, fractional distillation yielded purified acetonitrile. The purity was confirmed by the absence of color after adding 5 μL of Sanger's reagent to a 1 mL aliquot. Similar quality acetonitrile could be obtained by distillation over barium oxide and ninhydrin.

The purification of chlorobenzene was adapted from Monnery et al. (Angew. Chem. Int. Ed. 2018, 130 (47)). In short, chlorobenzene was purified in 500 mL batches by washing with 50 mL of sulfuric acid (98%) until no discoloration occurred after standing overnight (4 washes), followed by three 50 mL washes with water, saturated aqueous sodium carbonate and water. Next, chlorobenzene was dried with anhydrous magnesium sulfate (pre-dried at 180° C.). Subsequently, chlorobenzene was transferred to a distillation setup equipped with a fractional distillation column (40 cm), and the solution was refluxed for 2 hours over tosyl isocyanate (1 mL/100 ml of chlorobenzene) under inert atmosphere to remove any nucleophilic impurities. Finally, fractional distillation yielded the purified chlorobenzene.

Purification of 2-methyl-2-oxazoline, 2-ethyl-2-oxazoline and 2-propyl-2-oxazoline. The 2-oxazoline monomer was transferred to a distillation setup equipped with a fractional distillation column (40 cm), and the solution was refluxed over CaH₂ under inert atmosphere. After the evolution of H₂ gas had ceased (judged from a manometer, 2 hours), the monomer was fractionally distilled where the first 10% was discarded and the bulk was collected in a schlenck flask equipped with a three way tap and stir bar. Next, another distillation was performed in a similar setup, whereby 1 mL of distilled MeOTs was added to the monomer. The monomer was fractionally vacuum distilled, whereby the heating was carefully kept below 80° C. to prevent excessive polymerization.

DMF used for the NCA polymerization was distilled over tosyl isocyanate (1 mL/100 mL) and stored over molecular sieves and isocyanate beads (Biotage) at −30° C.

Cation exchange chromatography was performed on CM 50 sephadex.

Size-exclusion chromatography (SEC) at 40° C. using a Jasco HLC-8220 machine coupled with a refractive index (RI)-2031 detector and TSKgel HHR columns (G4000 and G3000) flowed with LiCl (10 mM) dissolved in DMF as an eluent solvent (flow rate 0.8 mL min⁻¹). The SEC analysis was performed with the JASCO ChromNav software and the analysis utilized PEG standards of known M_{w} (Agilent Technologies, Santa Clara, CA) as calibrants. NMR measurements were performed on a 400 MHz JEOL ECS 400; JEOL, Tokyo, Japan device. Chemical shifts of ¹H are reported relative tetramethylsilane. ¹⁹F chemical shifts are reported against the internal calibration of the machine. MALDI-TOF-MS measurements were performed a Bruker ultrafleXtreme, Bruker Daltonics, Bremen, Germany. Analyte solutions were prepared by mixing 10 μL of a 20 mg/mL solution of α-cyano-4-hydroxycinnamic acid (sigma Aldrich) with 4 μL of a 4 mg/mL polymer solution and 2 μL of a 2 mg/mL solution of NaTFA (Sigma Aldrich). Generally THF was utilized as the solvent, except for PMeOx samples, where MeOH had to be utilized as the solvent. Samples whereby the analyte contained a tert-butyl carbamate group made use of trans-2-[3-(4-tert-Butylphenyl)-2-methyl-2-propenylidene]malononitrile as a matrix. The analyte solutions were subsequently spotted on a stainless steel 96-well sample stage according to the dried droplet method. The same well was spotted at least twice with the analyte solution.

### Example 1-1

Procedure 1: Representative Polymerization of 2-Oxazoline Monomers Initiated with Pentafluorobenzyl Bromide

An oven dried (180° C.) schlenck flask equipped with a stir bar and three-neck tap was cooled down under vacuum (0.5 mbar) and subsequently filled with dry Argon. These vacuum-argon cycles were repeated 2 more times. Next, acetonitrile (9 mL) and EtOx (6 mL, 60 mmol) were added to the schlenck flask to obtain a 4 M monomer solution. Next, an appropriate amount pentafluorobenzyl bromide was added to the flask to obtain to a solution with the desired [monomer]:[initiator ratio]. This mixture was subsequently heated to temperatures ranging from 40-80° C., typically 70° C., and left to react until a conversion of 90% was reached as assessed by ¹H NMR. Next, the polymer was terminated with the appropriate nucleophile, which is described separately.

### Example 1-2

Procedure 2: Representative Polymerization of 2-Oxazoline Monomers Initiated with Pentafluorobenzyl Tosylate

An oven dried (180° C.) schlenck flask equipped with a stir bar and three-neck tap was cooled down under vacuum (0.5 mbar) and subsequently filled with dry Argon. These vacuum-argon cycles were repeated 2 more times. Next, chlorobenzene (9 mL) and EtOx (6 mL, 60 mmol) were added to the schlenck flask to obtain a 4 M monomer solution. Next, in a glove bag, an appropriate amount pentafluorobenzyl tosylate was added to the flask under inert atmosphere to obtain to a solution with the desired [monomer]:[initiator ratio]. This mixture was subsequently heated to temperatures ranging from 40-80° C., typically 70° C., and left to react until a conversion of 90% was reached as assessed by ¹H NMR. Next, the polymer was terminated with the appropriate nucleophile, which is described separately.

### Example 2-1

### Procedure 3: Azide Termination for Polymer Prepared by Procedure 1

Sodium azide (10 molar equivalents relative to initiator) was added to the colorless polymerization mixture, and the reaction mixture was left to stir 16 h at room temperature. Next, 10 mL of ethyl acetate was added, and the insoluble sodium azide was filtered off through a 0.44 μm pore-sized PTFE syringe filter. The filtrate was subsequently precipitated in 10-fold excess of ether, the precipitation was repeated once. Next, the ether was decanted and after most of the remaining ether had evaporated, hot water was added. Finally, the polymer was isolated as a white powder after freeze drying

### Example 2-2

### Procedure 4: Azide Termination for Polymer Prepared by Procedure 2

Tetrabutylammonium azide (2 molar equivalents relative to initiator) was added to the colorless polymerization mixture, and the reaction mixture was left to stir 16 h at room temperature. Next, 10 mL of ethyl acetate was added, and the polymer was subsequently precipitated in 10-fold excess of ether, the precipitation was repeated once. Next, the ether was decanted and after most of the remaining ether had evaporated, hot water was added. Finally, the polymer was isolated as a white powder after freeze drying.

### Example 2-3

### Procedure 5: Amine Termination for Polymer Prepared by Procedure 2

Piperidine (10 molar equivalents relative to initiator) was added to the colorless polymerization mixture, and the reaction mixture was left to stir 24 h at room temperature. Next, 10 mL of ethyl acetate was added. Next, the polymer was precipitated in 20-fold excess of ether, and the precipitation was repeated once after dissolving the polymer in ethyl acetate. Next, the ether was decanted and after most of the remaining ether had evaporated, hot water was added. Finally, the polymer was isolated as a white powder after freeze drying

### Example 2-4

### Procedure 6: Thiol Termination for Polymer Prepared by Procedure 2

Thiol and triethylamine (2 and 2.5 molar equivalents relative to initiator, respectively) were added to the colorless polymerization mixture, and the reaction mixture was left to stir 10 h at room temperature. Next, 10 mL of ethyl acetate was added and the polymer was precipitated in 10-fold excess of ether, and the precipitation was repeated once after dissolving the polymer in ethyl acetate. Next, the ether was decanted and after most of the remaining ether had evaporated, hot water was added. Finally, the polymer was isolated as a white powder after freeze drying

### Example 2-5

### Procedure 7: Carboxylic Acid Termination for Polymer Prepared by Procedure 1

Acetic acid and triethylamine (2 and 2.4 molar equivalents relative to initiator, respectively) were added to the colorless polymerization mixture, and the reaction mixture was left to stir 16 h at room temperature. Next, 10 mL of ethyl acetate was added. Next, the polymer was precipitated in 20-fold excess of ether, and the precipitation was repeated once after dissolving the polymer in ethyl acetate. Next, the ether was decanted and after most of the remaining ether had evaporated, hot water was added. Finally, the polymer was isolated as a white powder after freeze drying

### Example 3-1

### Procedure 8: Para-Fluoro Azide Substitution

In short, 0.2 g of PFP-PEtOx₄₅—N₃ (0.05 mmol of PFP groups) was dissolved in 2 mL N,N-dimethylformamide (DMF) together with 2.5 equivalents of sodium azide (8.2 mg, 0.125 mmol) relative to the PFP-moiety and heated for 2 hours at 80° C. Next, the reaction mixture was cooled down and the polymer was precipitated in 10-fold excess diethylether. Next, the polymer was dissolved in 2 mL ethyl acetate, and the excess of sodium azide was removed by filtering the mixture through a 0.2 μm pore syringe filter. The obtained filtrate was precipitated again in 20 mL diethylether. The supernatant was decanted and the polymer was subsequently dissolved in water and freeze dried.

### Example 3-2

### Procedure 9: Para-Fluoro Thiol Substitution

Example: In short, 0.2 g of PFP-PEtOx45-N₃ (0.05 mmol of PFP groups) was dissolved in 2 mL NNdimethylformamide (DMF) together with either 5 equivalents of thiol and 6 equivalents of triethylamine, 5 equivalents of thiol and 4 equivalents of KOtBu, or 2 equivalents of thiol and 1.8 equivalents of DBU relative to the PFP-moiety. In case triethylamine was employed as the base, the mixture was heated to 60° C. for at least 16 h. In case the other bases were employed, the reaction mixture was left to stir for 1 h at room temperature. After the reaction had reached completion, the polymer was precipitated in 10-fold excess diethylether. The supernatant was decanted and the polymer was subsequently dissolved in water and freeze dried.

### Example 3-3

### Procedure 10: Para-Fluoro Amine Substitution

Example: In short, 0.2 g of PFP-PEtOx₄₅—N₃ (0.05 mmol of PFP groups) was dissolved in 2 mL NMP together with either 7 equivalents of amine relative to PFP groups, whilst maintaining an argon atmosphere. In case ethylenediamine was selected as the amine, 15 equivalents were employed in order to prevent polymer coupling reactions. Next the reaction mixture was heated to 70° C. for at least 16 h. After the reaction had reached completion, the polymer was precipitated in 10-fold excess of diethylether. The supernatant was decanted and the polymer was subsequently dissolved in water and freeze dried.

### Example 3-4

### Procedure 11: POx-Lipid Synthesis

Example: In short, 0.2 g of 1-thioglycerol-TFP-PEtOx45-N₃ (0.10 mmol of hydroxyl groups, 1 equivalent) was dissolved in 2 mL DCM and 5 equivalents of the fatty acid and 5 equivalents of diisopropylcabrodiimide and 0.05 eq of DMAP were added. The reaction mixture was stirred at room temperature for at least 16 hours, after which the polymer was isolated by precipitation in a 10-fold excess of ether. Next the polymer was redissolved in DCM and passed over a neutral aluminum oxide plug, to remove excess of fatty acid. Next the polymer solution was added to benzene and subsequently freeze dried to afford a white powder. In the case PMeOx-lipids were synthesized, the polymer was dispersed in water after benzene freeze drying, and the solution was added to AMICON diafiltration tubes with a molecular weight cut-off of 10 kDa and centrifuged to remove proton-initiated impurities. The diafiltration by centrifugation was repeated three times by diluting the remaining solution by 10 times.

### Example 3-5

### Procedure 12: Para-Fluoro Alkoxide Substitution

Example: In short, 0.2 g of PFP-PEtOx₄₅—N₃ (0.05 mmol of PFP groups) was dissolved in 2 mL NMP together with 3 equivalents of phenol and 3 equivalents of TBD relative to PFP groups, whilst maintaining an argon atmosphere. Next the reaction mixture was stirred for 24 h at room temperature. After the reaction had reached completion, the polymer was precipitated in 10-fold excess of diethylether. The supernatant was decanted and the polymer was subsequently dissolved in water and freeze dried.

### Example 4

### POX Liposome Preparation and Characterization

POX-lipid containing liposomes were prepared using a thin film hydration method.⁶⁷ Briefly, DOPC, cholesterol and C18-MeOX or C18-PtOX were separately dissolved in chloroform and then mixed in a molar ratio of (62.5:32.5:5). The chloroform was evaporated and the lipid film was hydrated using 37° C. 10 mM HEPES buffer (pH 7.4) to give a final total lipid concentration 5 mM. The liposomes were then extruded multiple times using 200 and 100 nm NanoSizer MINI Extruder (T&T Scientific). Particle size was measured using dynamic light scattering (DLS) using a diode laser (λ=532 nm) with a scattering angle of 173° (Zetasizer Nano-ZS, Malvern Instruments, Worcestershire, UK). For blood circulation studies, the DiD fluorescent probe was added to the lipid mixture to give a final concentration 0.03 mM in the liposomal suspension. DiD-liposomes were diluted 10× in PBS and 200 μL were injected to Balb/C mice by a tail vain injection.

### Example 5

### IVCLSM Studies

Blood circulation of POX-lipid containing liposomes was measured using Intravital confocal laser scanning microscopy (IVCLSM). A system equipped with a 20×objective lens, a 640-nm diode laser, and a band-pass emission filter of 700/75 nm was used. The pinhole was set to produce a 10-μm diameter optical slice. 5-week-old female Balb/C mice ((The Jackson Laboratory Japan, Inc.) were placed on a microscope stage, and the DiD signal in earlobe dermis vessels was observed soon after tail vein injection of liposomes. All animal experimental protocols followed the guidelines of the Innovation Center of NanoMedicine (iCONM), Kawasaki Institute of Industrial Promotion. Fluorescence images were recorded every 4 sec for the first 3 min post injection, followed by snapshots every 1 min. The fluorescence intensity was calculated as the intensity of a region of interest (ROI) of the vein minus the background. The relative fluorescence intensity was calculated as a ratio of the fluorescence intensity value each time point relative to the maximum intensity value throughout the measurement. Elimination and distribution half-lives were calculated by compartmental analysis using the Prism GraphPad software.

### Example 6

mRNA Synthesis

DNA encoding full length SARS-CoV-2 spike protein with 120 adenine nucleotides added to the 3′ end and BsmBI cut sites was cloned into pSP73 plasmid vector by Genscript Japan (Tokyo, Japan). The plasmid was amplified in E. coli DH5a competent cells (Takara Bio Inc., Otsu, Japan), and then extracted and purified using a Nucleobond xtra maxi plus EF kit (Takara, Japan). Plasmids were linearized and then fragmented by incubation with BsmBI overnight at 55° C., and the desired DNA fragment was separated by gel electrophoresis and extracted using a gel extraction kit (Qiagen, Hilden, Germany). The extracted DNA was further treated with T4 DNA polymerase (Takara Bio Inc., Otsu, Japan) to obtain blunt-end DNA. Finally, in vitro transcription was carried out using the mMESSAGE mMACHINE™ kit (Thermo Fisher Scientific). The reaction was allowed to proceed at 37° C. for 14 h, and the transcribed mRNA was purified using RNeasy mini-Kit (Qiagen, Hilden, Germany). The quality of mRNA was checked using a Bioanalyzer (Agilent Technology, CA, USA).

### Example 7

mRNA-LNP Preparation and Characterization

An ionizable lipid mRNA-LNP was formulated by microfluidic mixing. One volume of ethanol containing cholesterol, DSPC, D-Lin-MC3-DMA and a polymer-conjugated lipid was mixed with 3 volumes of 50 mM sodium citrate buffer (pH=3) containing the mRNA using an Ignite microfluidic micromixer (Precision NanoSystems Inc., Vancouver, BC, Canada), at a 12 mL/min flow rate. The product was then diluted 40× in PBS and concentrated using 30 kD Amicon centrifugal filters (Millipore, MA, USA) to remove residual ethanol. Encapsulation efficiency was determined using Quant-it™ RiboGreen RNA Assay Kit (Thermo Fisher Scientific, Waltham, MA, USA). Particle size was measured as described above.

### Example 8

### IVIS Studies

Fluc mRNA-LNPs formulated using a conventional PEG-lipid or one of the new POX-lipids were intravenously injected at a dose of 5 μg mRNA per mouse (Balb/C, female, 5 weeks). 4 and 24 h post injection, mice were intraperitoneally injected with 200 μL of 15 mg/mL luciferin substrate (Promega) 10 min prior imaging. Mice were then placed on temperature-controlled stage in the in vivo imaging system (IVIS, PerkinElmer) equipped with O₂ and isoflurane for anesthesia. Imaging exposure time was set at 10 sec and the total flux of luminescence was measured by gating ROI at the abdomen to quantify protein expression in the liver. Following whole body imaging at 24 h, mice were sacrificed and important organs were harvested and imaged after submerging in luciferin in a petri dish. Each organ was gated by a separate ROI and used to calculate protein expression distribution.

### Example 9

### Immunization Studies

Balb/C mice (female, 5 weeks) were injected 5 μg spike mRNA-LNP in the thigh muscle, followed by an additional 5 μg booster dose 3 weeks after. Two weeks after the last dose, blood was collected in heparinized tubes and mice were sacrificed to harvest the spleens for the quantification of humoral and cellular immunity, respectively. Blood plasma was obtained by spinning the blood at 2000×g for 10 minutes in a refrigerated centrifuge and kept at −80 C until used.

Plasma anti-spike IgG were quantified using enzyme linked immunosorbent (ELISA). First, SARS-CoV-2 Spike S1+S2 recombinant protein (SinoBiological) was mounted onto a clear flat-bottom Immuno Nonsterile 96-Well Plates (Thermo) by incubation overnight at 4° C. After washing out excess protein, 50 μL of diluted plasma samples was added to each well and incubated overnight at 4° C. Next, goat anti-mouse IgG-HRP (R&D systems, 1:8000 dilution) was added to each well after washing and incubated for additional 2 hours at 23° C. Finally, 100 μL/well of the HRP substrate was added and incubated for 30 min at 23° C. away from light. The reaction was stopped by the addition of 2M sulfuric acid and absorbance at 492 nm was recorded using a plate reader (Tecan, Switzerland).

For the quantification of spike protein-specific INF γ-producing T cells in the spleens of immunized mice, spleens were first disintegrated using a steel grid mesh in the presence of 5 mL of RPMI-1640 medium containing 10% FBS, 1 mM sodium pyruvate, 10 mM HEPES, 50 μM mercaptoethanol and 1% penicillin/streptomycin. The suspension was then passed through a 40 μm nylon mesh (Cell strainer, Falcon) to form a single-cell suspension. Splenocytes were seeded at a density of 2.5×10⁵ cell/well in an anti-IFNγ ELISpot plate and stimulated by the addition of 0.025 μg/well whole spike epitope mixture (JPT Peptide Technologies). The plates were incubated at 37° C. and 5% CO₂ overnight, washed and treated according to the manufacturer's protocol. The developed spots were counted on an ELISpot plate reader (AID GmBH, Germany).

### Example 10

### Toxicity Studies

Biochemical markers in blood plasma were evaluated following 4 and 24 h post intravenous injection of 5 μg Fluc mRNA- LNP. Samples were measured using Model 7180 Automatic Analyzer Hitachi High-Technologies Corporation performed by ORIENTAL YEAST CO., LTD. (Tokyo, Japan) JCA-BM6050 automatic analyzer (JEOL, Tokyo, Japan). Aspartate aminotransferase (AST), alanine aminotransferase (ALT), alkaline phosphatase (ALP) and lactate dehydrogenase (LDH) levels were measured using the Japan Society of Clinical Chemistry (JSCC) Recommended method. Creatinine (CRE) was measured using the Creatininase-HMMPS method, and uric acid (UA) levels were determined by the Uricase-HMMPS method was measured using the creatinine amidohydrolase-creatinine amidinohyrolase-SOX-POD enzymatic method, and b. Blood urea nitrogen (BUN) was determined by the Urease-GLDH enzymatic method. Uric acid (UA) levels were determined by an enzymatic method.

### Example 11

### Statistical Significance

Statistical significance between two groups was analyzed using an unpaired, two-tailed Student's t test. For comparison against nontreated samples, non-repeated ANOVA followed by Dunette's test was used. A statistically significant difference was set at p<0.05.

### Results and Discussion

Polymers bearing pentafluorophenyl (PFP)-groups, such as poly(pentafluorostyrene) or poly(pentafluorobenzyl acrylates), have been shown to be reactive to a wide range of substrates, reacting with high efficiency with a variety of N, O and S-nucleophiles in a single reaction trajectory, i.e. a para-fluoro substitution. This chemistry, therefore, would be an attractive approach for the a-end-group diversification of 2-oxazolines, on the condition that the PFP-groups are unreactive towards the N, O and S-nucleophiles employed in the termination step of the cationic ring-opening polymerization (CROP). It should be noted, however, that comprehensive studies of the termination of the 2-oxazolinium species under conditions relevant to the polymerization have not been described, and that the relative reactivity of the PFP and 2-oxazolinium groups is presumably dependent on the employed nucleophile. An additional complication is that the termination step of the CROP typically requires the addition of excess of the N-, O- or S-nucleophiles to ensure quantitative reaction, as well as avoiding undesired side-reactions, such as overalkylation in the case of amines. So far, only hydroxide species, which can be applied in an equimolar ratio relative to the 2-oxazolinium species, have been employed as a termination agent for pentafluorobenzyl bromide initiated PEtOx, though selectivity was not confirmed by ¹⁹F NMR.

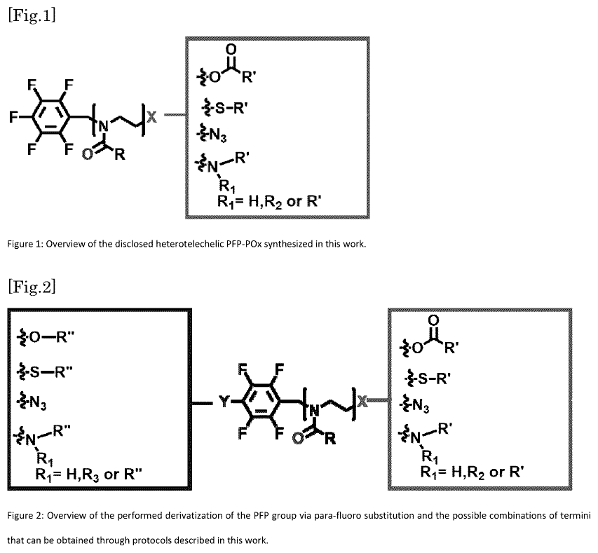

Hence we investigated the termination of pentafluorobenzyl bromide/tosylate initiated 2-oxazoline polymerizations under different experimental conditions (reaction time, temperature and solvent), to enable selective termination of the living 2-oxazolinium chain end with different N—, O-, and S-nucleophiles, consequently affording heterotelechelic POx bearing the PFP-moiety at the a-end, and azide, amine, ester or thioether groups at the ω-end as displayed in FIG. 1. In addition, we also describe the derivatization of these heterotelechelics via para-fluoro substitution to obtain novel heterotelechelic combinations, as depicted in FIG. 2.

### Selective Azide Termination

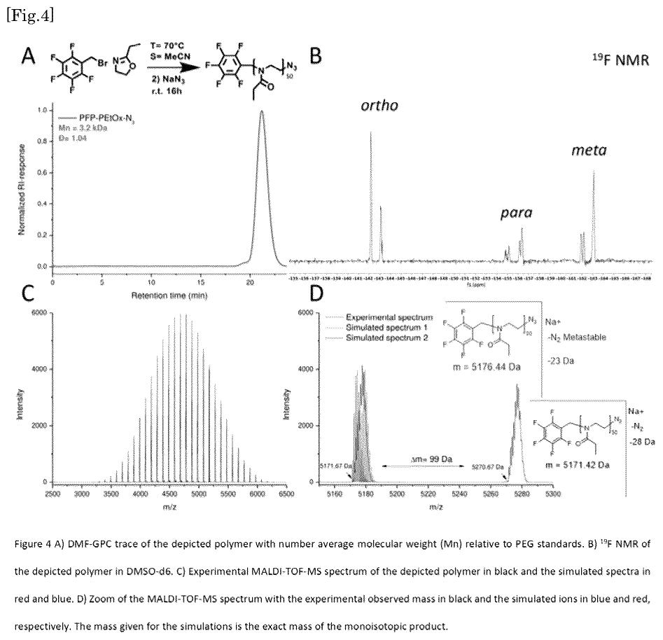

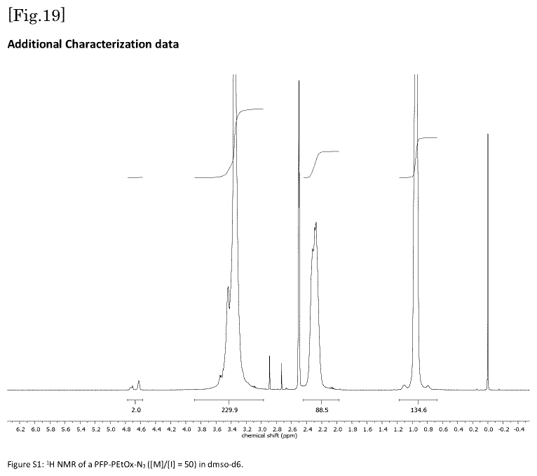

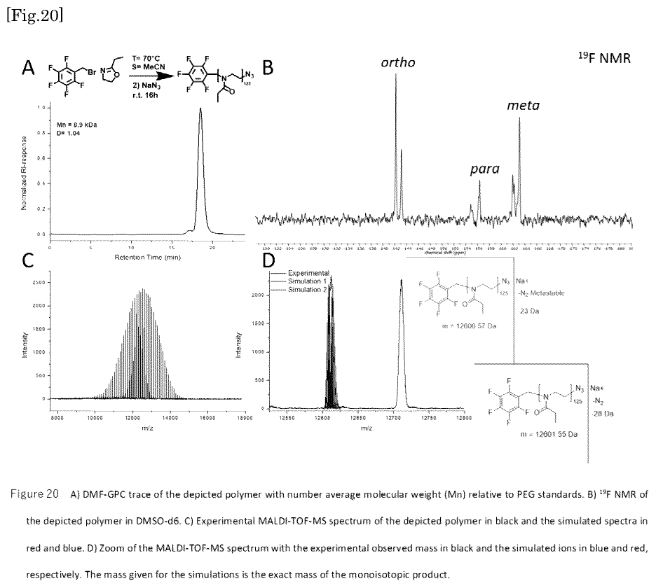

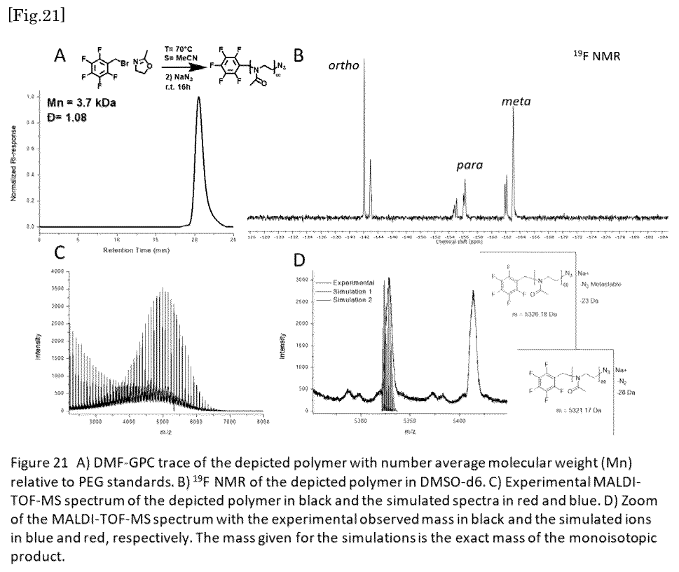

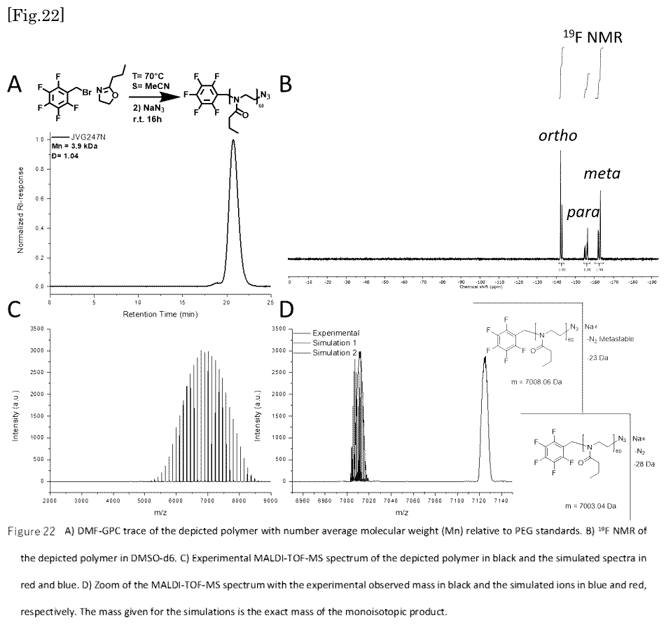

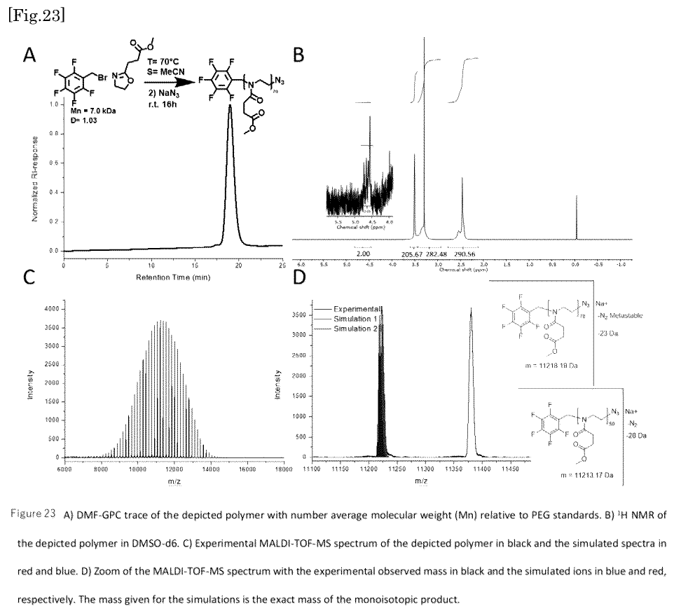

First we investigated the selective termination of the living polymer chains by sodium azide, as both the 2-oxazolinium and the PFP moieties have been shown to efficiently react with the azide anion upon mild heating in dipolar aprotic solvents, thus presenting an ideal starting point to probe the differential reactivity of both electrophiles. Towards this end, the polymerization of 2-ethyl-2-oxazoline (EtOx) was initiated with pentafluorobenzyl bromide in acetonitrile (MeCN), and the polymerization was allowed to proceed at 70° C. until a monomer conversion of >90% was reached. Next, 10 molar equivalents of sodium azide relative to the initiator were added to the polymerization mixture and the mixture was left to stir at room temperature and periodically sampled to monitor the conversion of the PFP moiety by ¹⁹F NMR. As can be seen from FIG. 3, after 16 h the characteristic ¹⁹F signals for the intact PFP moiety can be seen in the NMR, please note that splitting of the signal occurs due to slow rotation of the amide bond on NMR time scale, and as the reaction time increases, peaks corresponding to the tetrafluorophenyl azide appear. Nevertheless, these results indicate that the product with an intact PFP moiety can be isolated by controlling the reaction time. This was also confirmed by isolating polymers with different degrees of polymerization (DP) after stirring 16 h in the presence of NaN₃, as can be seen from FIG. 4 for a poly(2-ethyl-2-oxazoline) (PEtOx) prepared with an [M]/[I]=50. FIG. 4A shows that the obtained polymer possesses a narrow molecular weight distribution with)=1.04, which is in line with dispersity values expected for quasi-living ring-opening polymerizations, while FIG. 4B demonstrates that the terminal PFP-groups have remained intact. This is further supported by the MALDI-TOF-MS measurements in FIG. 4C-D, where a monomodal mass distribution is obtained, whereby the experimental spectrum closely matches the simulated spectra. It should be noted that the terminal azide end-group is prone to fragmentation in MALDI-TOF-MS, whereby the expulsion of N₂ leads to molecular ions with −23 Da and −28 Da difference relative to the intact molecular ion, whereas the latter was not observed. Also, note the absence of H-initiated species or other products in MALDI-TOF-MS, which indicates a high end-group fidelity of the synthesized PEtOx. The presence of the pentafluorobenzyl group was further confirmed via ¹H NMR (FIG. 19), due to the characteristic benzyl signals appearing at 4.55-4.75 ppm, whereby the relative ratio of the integral of polymer backbone CH₃ and pentafluoro benzyl protons (135/3=45) exactly matches the DP of the most intense signal observed in MALDI-TOF-MS, m=4682.12 Da, which again confirms the absence of side reactions, and the high purity of the obtained product. Similarly, this kinetic control could also be applied for the synthesis of heterotelechelics of EtOx with higher DPs, as well as 2-methyl-2-oxazoline (MeOx), 2-propyl-2-oxazoline (PrOx) and 2-methoxycarbonylethyl-2-oxazoline (C2MestOx) as can be seen from FIGS. 20-23. Though it should be noted that for MeOx, the presence of H-initiated species was observed in MALDI-TOF-MS and ¹H NMR, which can be attributed to the intrinsically higher chain transfer constants for this monomer.

### Para-Fluoro Azide Substitution

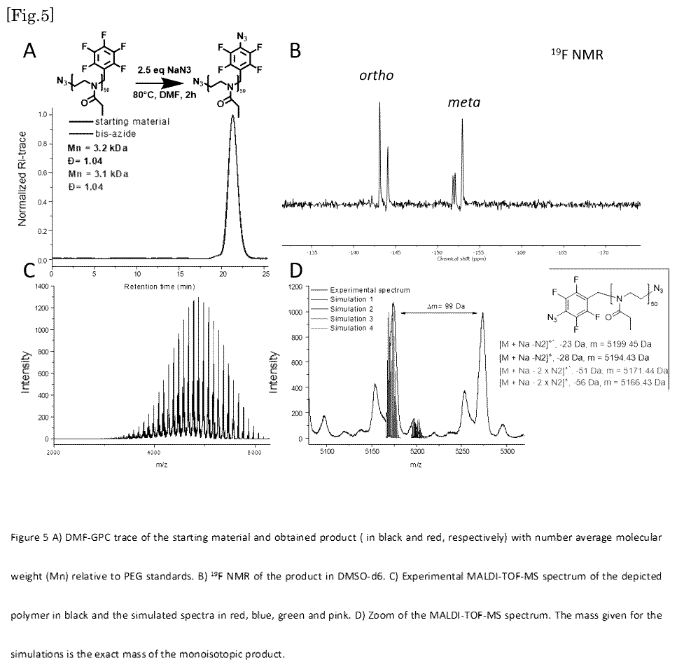

With these heterotelechelic polymers in hand, we subsequently investigated the para-fluoro substitution reaction on these substrates. First, the azide fluoro-substitution was performed in order to further prove the purity of the polymers synthesized in the previous section. Here, we adjusted the conditions reported by Noy et al. (Macromolecules 2019, 52(8), 3083-3091.) to obtain a PEtOx with two azide end-groups, viz. an alkyl azide and a tetrafluorophenylazide terminus. The transformation proceeded in a controlled fashion, as the DMF-GPC (FIG. 5A) showed a comparable molecular weight distribution to that of the starting material, with no evidence of chain coupling reactions. Furthermore, the ¹⁹F NMR clearly showed only two ¹⁹F populations, indicating that indeed the fluor in para-position was solely consumed, and that para-substituted tetrafluorphenyl azides were formed, which was also evident from the difference in chemical shift of the meta-fluorines (FIG. 5B). Finally, MALDI-TOF-MS further supported the formation of the product, as the peaks corresponding to the expected fragmentation products were present in the experimental spectrum(FIG. 5C-D). It should be noted however, that additional product peaks were present, which could not be identified. These peaks presumably arise from the photochemical transformation of the para-substituted tetrafluorphenyl azides, which is induced by the laser irradiation under the experimental conditions, thus yielding highly reactive nitrenium ions which can react with the matrix. Nevertheless, these results further confirm that selective azide termination is possible, as well as demonstrate that highly reactive para-substituted tetrafluorphenyl azides can be formed, which could be potentially employed in photochemical transformations or in azide-alkyne cycloadditions.

### Para-Fluoro Thiol Substitution

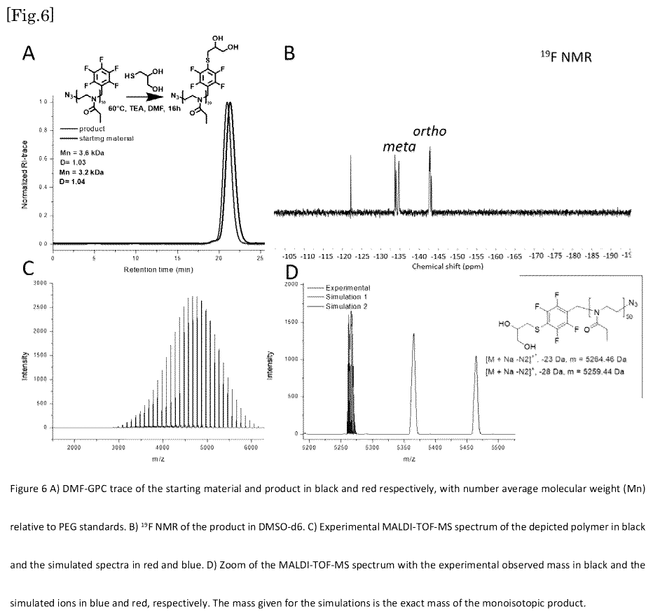

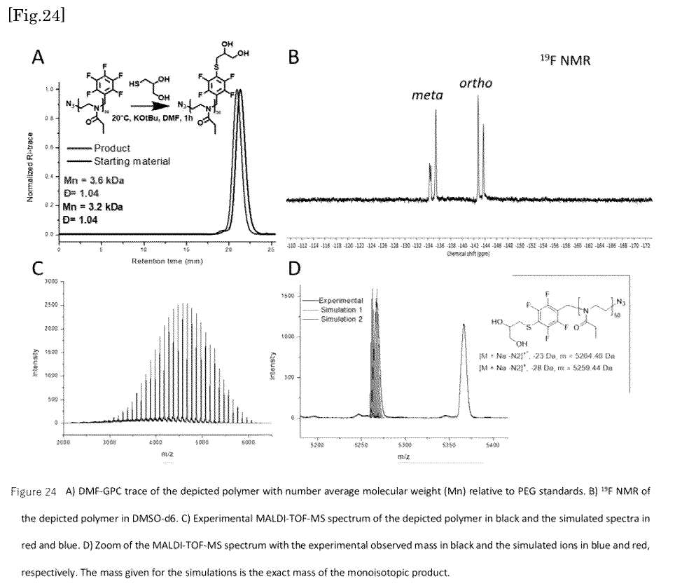

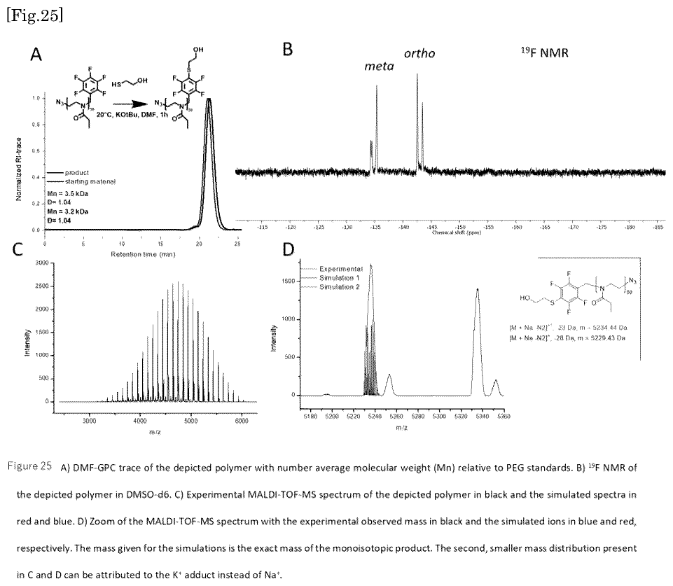

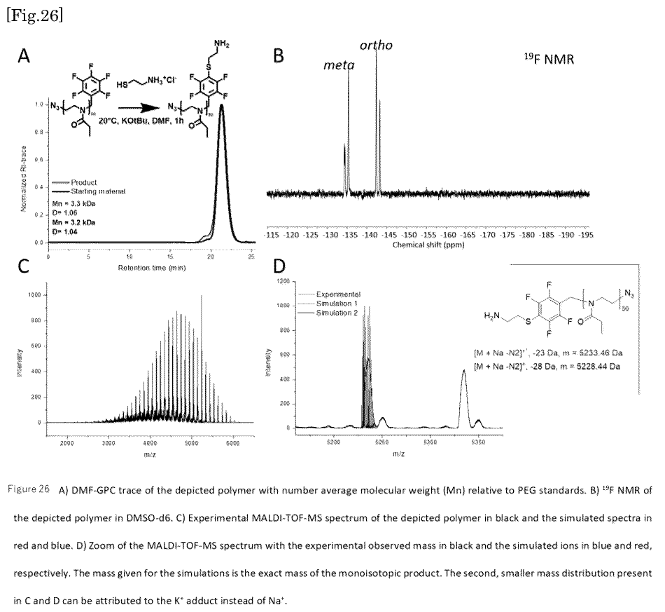

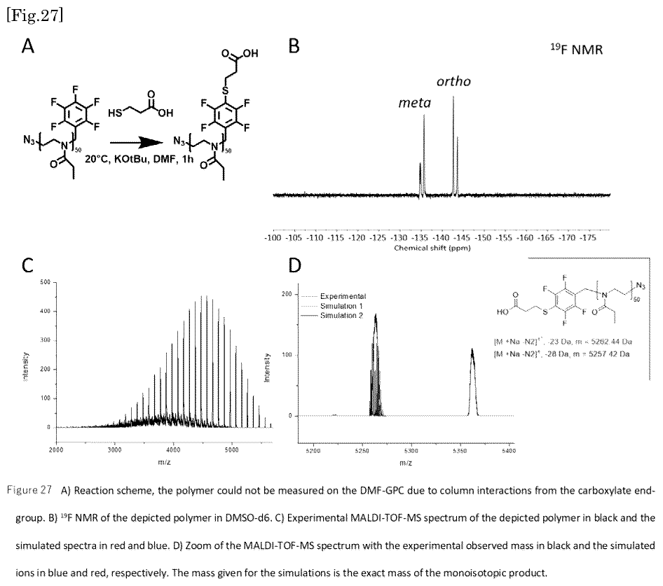

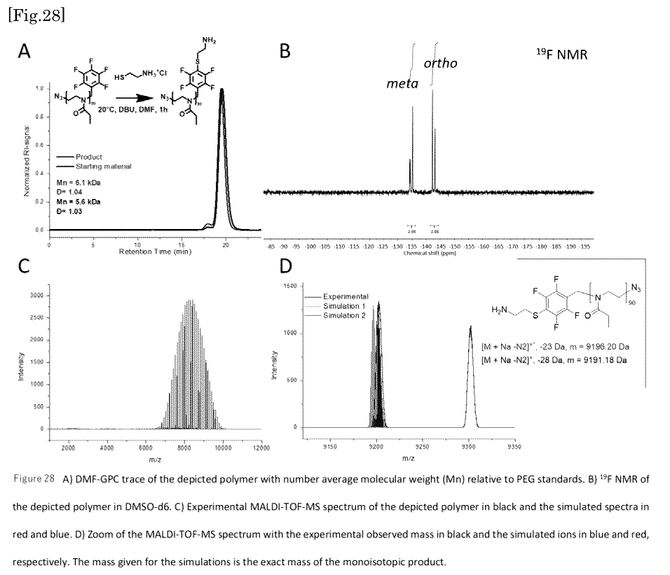

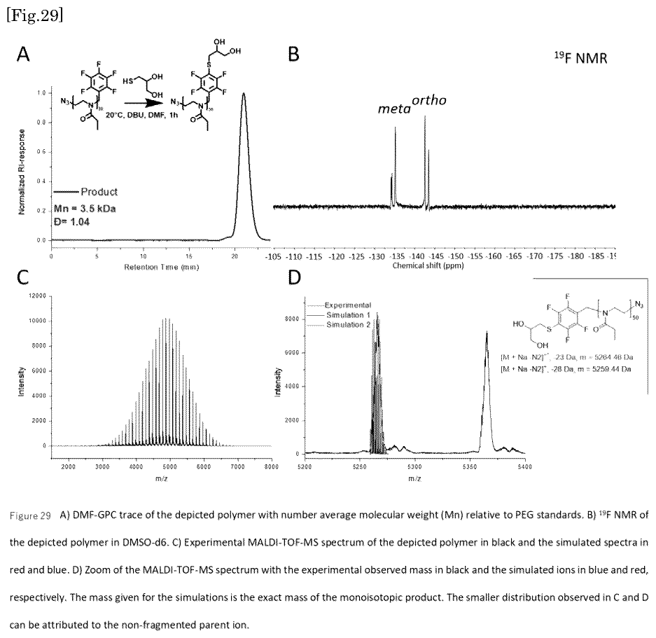

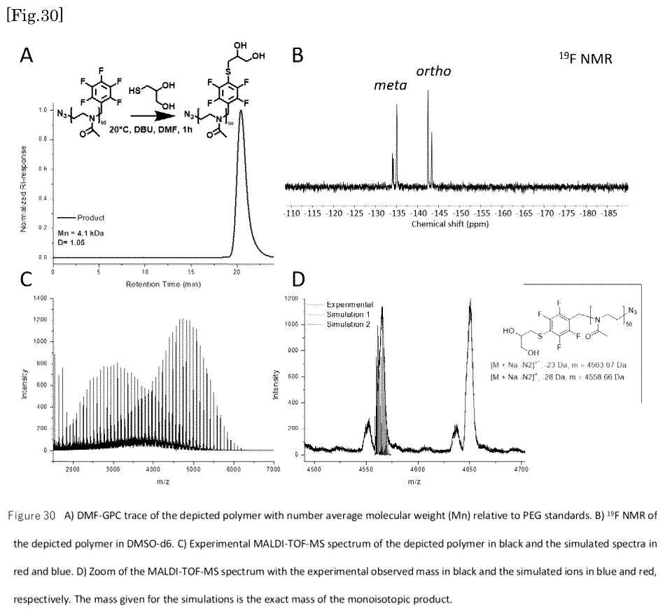

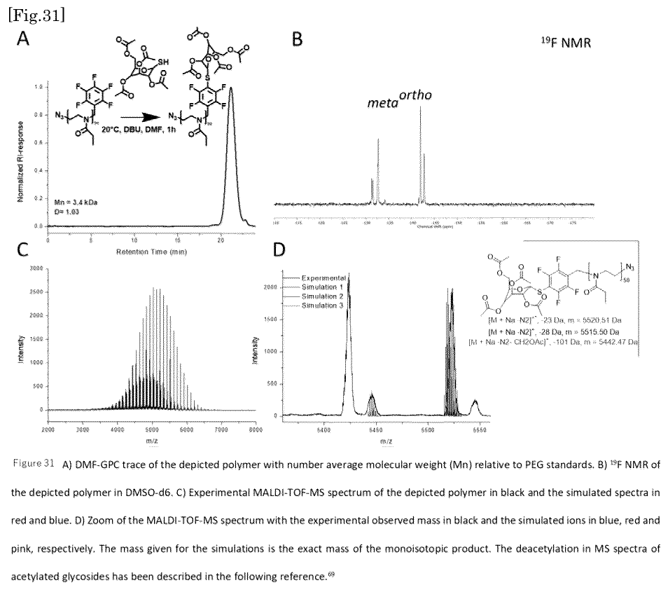

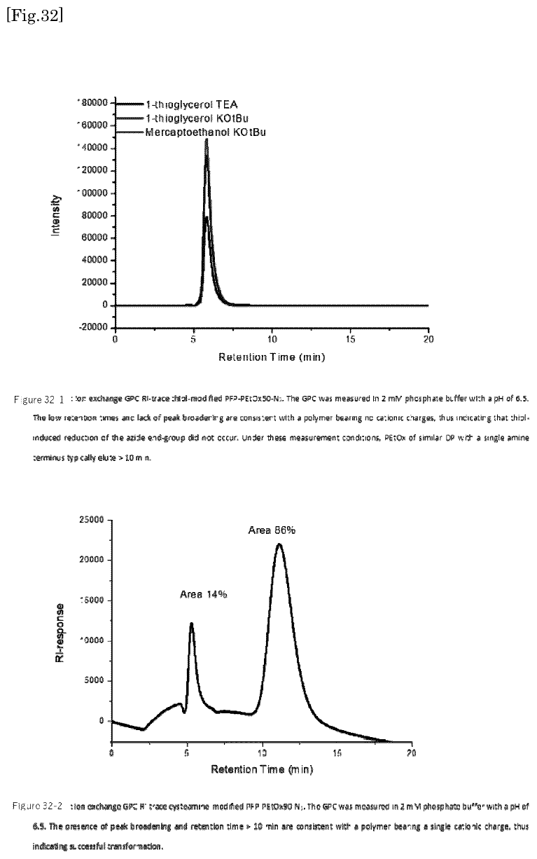

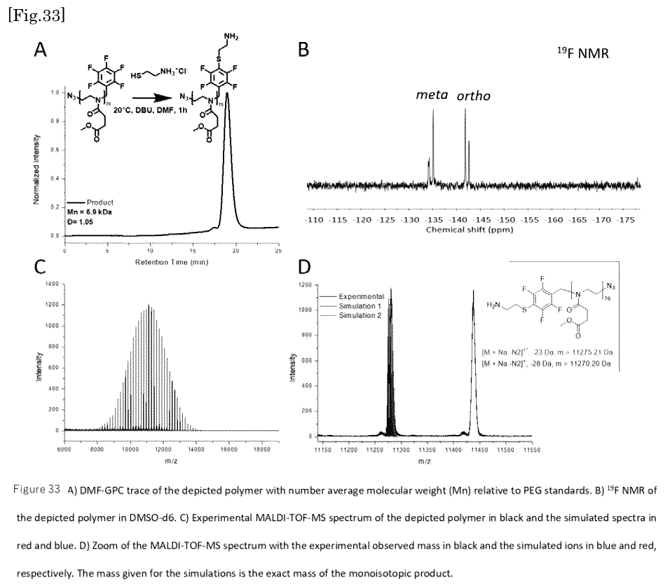

Next, we focused on the para-fluoro thiol substitution as a large variety of commercially available thiols could potentially be coupled under relatively mild conditions. This reaction is typically base catalyzed, whereby the formed thiolate attacks the PFP moiety in para-position. First, we explored the use of triethylamine as a base in this reaction for the coupling of aliphatic thiols. 1-thioglycerol was chosen as a model compound, as the obtained product could be used as a precursor for lipid-conjugates, vide infra. When triethylamine was employed as a base, significant excess of base and thiol were required (6 and 5 equivalents relative to PFP) and the reaction needed to be heated to 60° C. for 16 hours in DMF. Nonetheless, the reactions were successful as the characteristic product peaks could be seen in MALDI-TOF-MS and ¹H and ¹⁹F NMR, without noticeable changes in the molecular weight distribution besides the reduction in retention time in GPC as shown in FIG. 6A-D. Although successful, we attempted to reduce the reaction temperature and time by employing KOtBu as a base (Table 1). Under these conditions, effective transformation took place within 1 hour at room temperature by employing 5 equivalents of thiol and 4 equivalents of base. Unfortunately, attempts to reduce the molar excess of base and thiol were unsuccessful, as the reaction did not reach full conversion after 4 days. Hence, the use of DBU as a base was explored, which enabled full conversion of the PFP moiety within 1 hour at room temperature with as little as 1.35 equivalents of DBU and 1.5 equivalents of thiol. Nevertheless, the screening of different thiols revealed that 2 equivalents of thiol and 1.8 equivalents of DBU presented optimal conditions for a broad variety of thiols, including mercaptoethanol, cysteamine, mercaptopropionate and 1-thioglucose tetra acetate (FIGS. 24-31). Of note is that thiolates can also reduce alkyl azides, however under the employed reaction conditions no reduction towards amines was noted via ion exchange chromatography (FIG. 32-1), suggesting that the reduction is kinetically not relevant under these conditions. The polymers modified with cysteamine, however, did show retention profiles indicative of a cationic charge on the polymer, which confirms the successful transformation (FIG. 32-2). Furthermore, this transformation could also be applied on P(C2MestOx), thus demonstrating orthogonality to ester functional groups (FIG. 33).

TABLE 1
optimization of para-fluoro thiol substitution in terms
of excess of base, 1-thioglycerol and reaction time.

| | Thiol | Reaction time | | |
|---|---|---|---|---|
| Base equiv. | equiv. | to full conversion | | temperature |
| 6 equiv. TEA | 5 | <16 | h | 60° C. |
| 4 equiv. KOtBu | 5 | <1 | h | 20° C. |
| 1.4 equiv. KOtBu | 1.5 | >4 | days | 20° C. |
| 1.1 equiv DBU | 1.2 | >1 | h | 20° C. |
| 1.35 equiv DBU | 1.5 | <1 | h | 20° C. |
| 1.8 equiv DBU | 2 | <1 | h | 20° C. |

| | | | |
|---|---|---|---|
| 2.7 equiv DBU | 3 | <1 h, minor | 20° C. |
| | | presence of | |
| | | pluri-substituted | |
| | | products | |
| 3.6 equiv DBU | 4 | <1 h, minor | 20° C. |
| | | presence of | |
| | | pluri-substituted | |
| | | products | |

### Para-Fluoro Amine Substitution

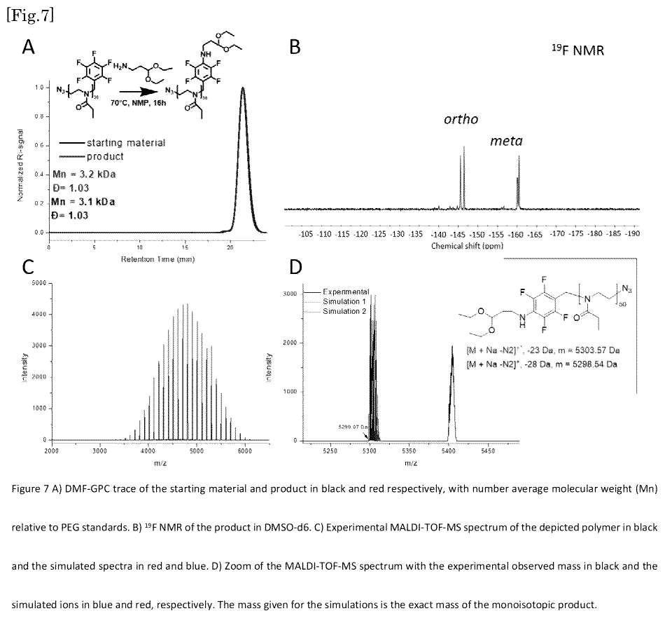

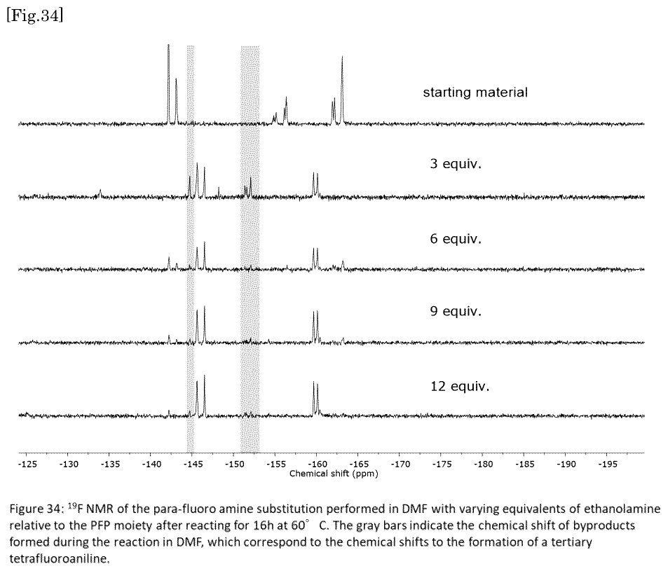

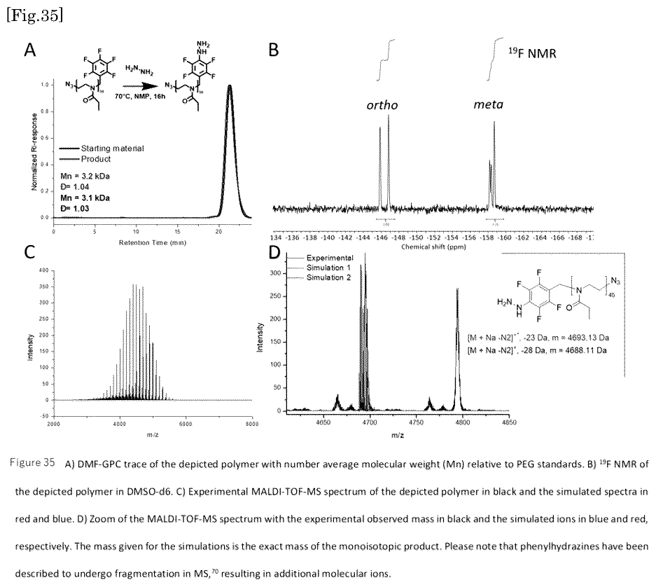

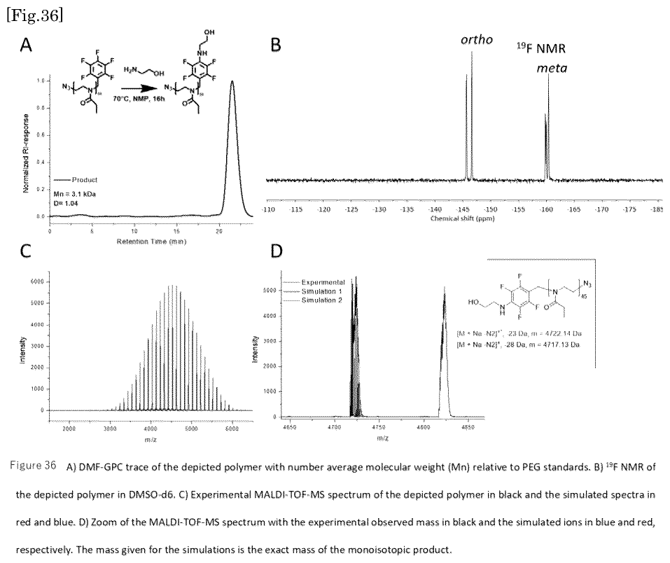

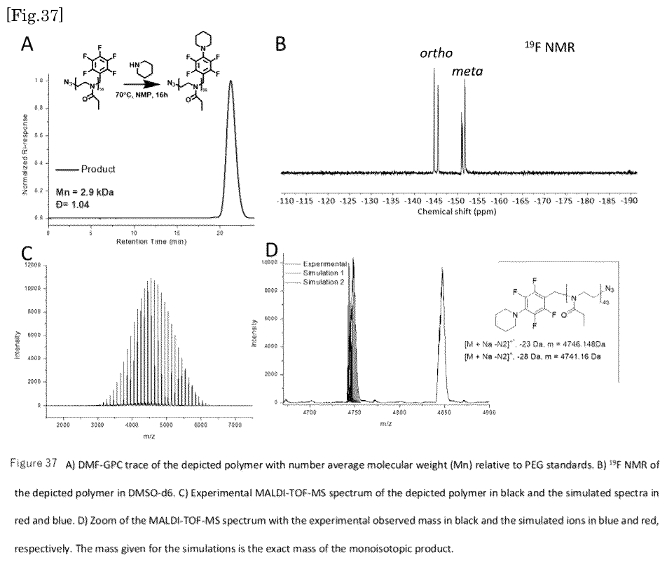

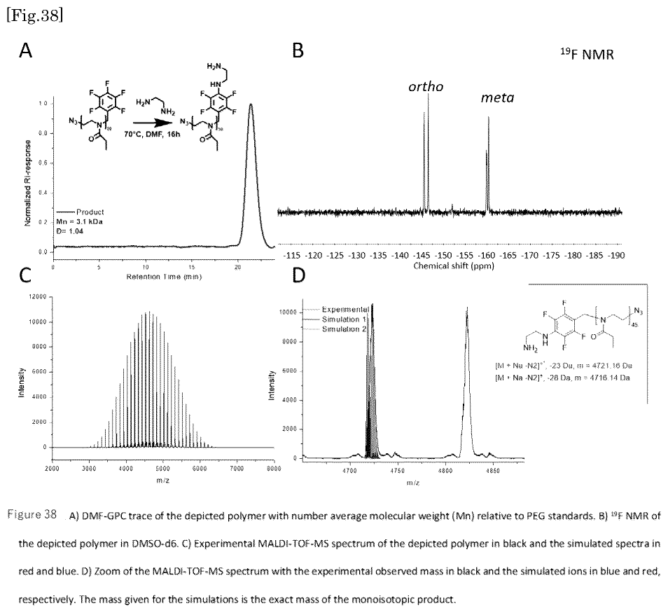

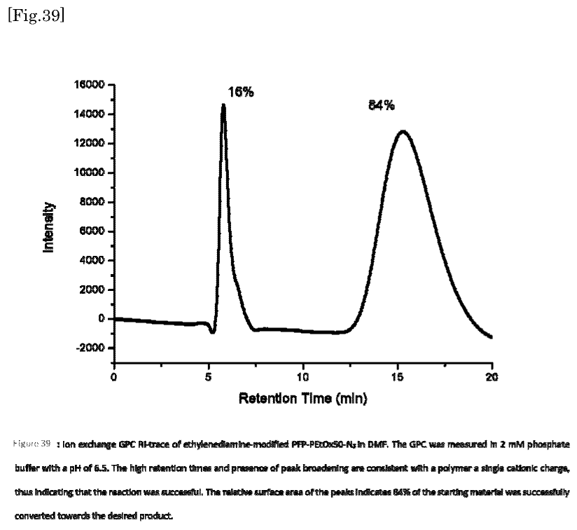

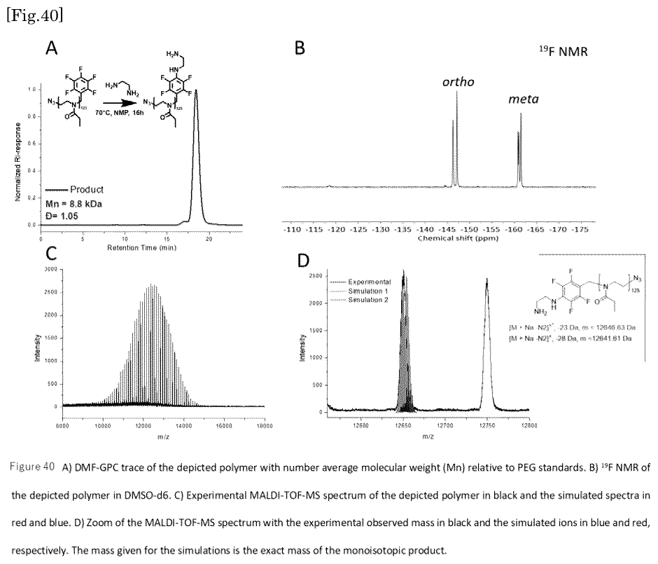

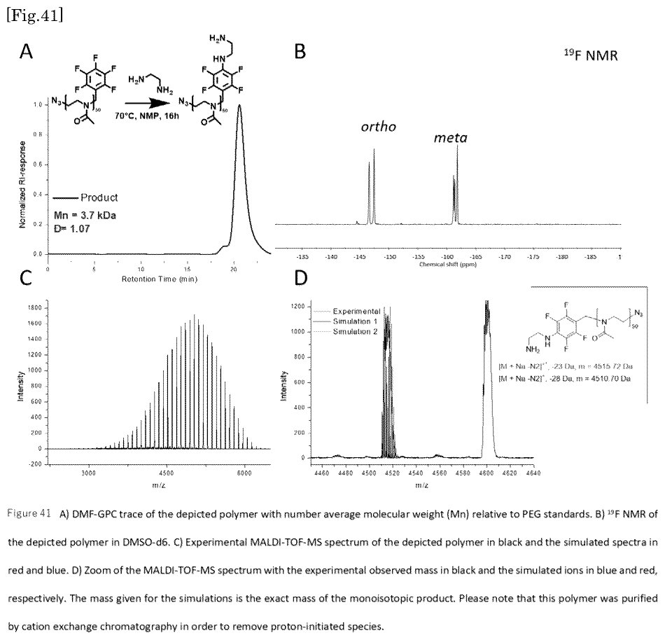

Having demonstrated the successful modification with thiols and azides, we subsequently explored the para-fluoro substitution with various amines. Initially, we adopted the conditions of Noy et al., however, it became apparent that these conditions went paired with significant formation of side-products. A screening experiment suggested that the side-products were formed as a result of the thermal and base induced composition of DMF, whereby the in situ generated N,N-dimethylamine effectively competed with the desired amine (FIG. 34). Hence, N-methylpyrrolidone (NMP) was selected as a more suitable solvent, thus enabling the efficient introduction of a large variety of amines, including piperidine, ethanolamine, ethylenediamine, 3-aminopropionaldehyde diethylacetal and hydrazine. In FIG. 7, the characterization for the 3-aminopropionaldehyde diethylacetal substituted polymer is shown as a representative example, while the other examples are shown in FIGS. 35-41.

### Para-Fluoro Alkoxide Substitution

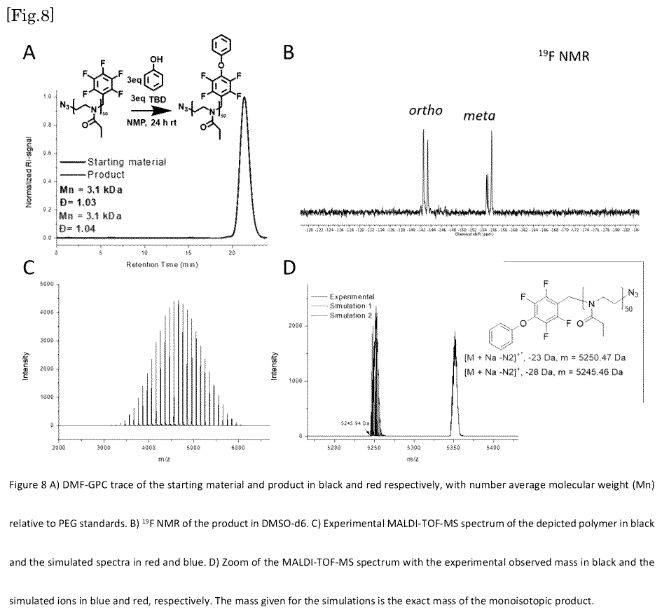

Finally, alkoxides were explored as a last set of substrates in the para-fluoro substitution. Here, phenol was chosen as a model compound, whereby the corresponding alkoxide was generated by in situ deprotonation with triazabicyclodecene (TBD) as a base. The reaction was shown to proceed with mild stoichiometric excess at room temperature in NMP for 24 hours. As is evident from the data in FIG. 8, the transformation proceeded smoothly without noticeable side-reactions, as the DMF-GPC still showed the characteristic narrow molecular weight distribution, while the¹⁹-F NMR showed the expect signals corresponding to the ortho- and meta-fluorines. Finally, the MALDI-TOF-MS spectra provide conclusive proof that the desired compound was obtained, further demonstrating the absence of side-reactions.

### Selective Carboxylic Acid Termination

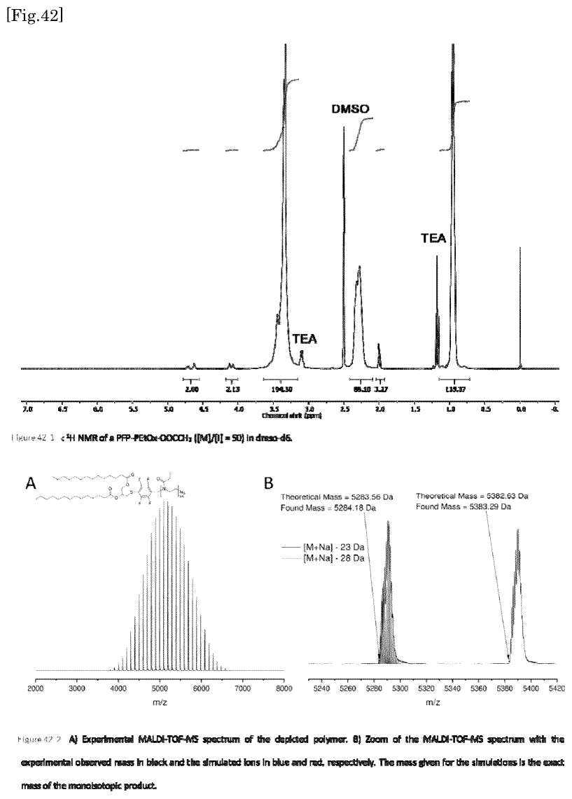

Besides azide anions as terminating agents for the CROP of 2-oxazolines, we also explored the termination with deprotonated carboxylic acids, to yield polymers with an esters at the ω-terminus. By simply adding 2 equivalents of acetic acid and 2.4 equivalents of triethylamine relative to the initiator to the polymerization mixture in MeCN, and stirring the mixture for 16 hours, the desired polymer could be isolated. As shown in FIG. 9, a polymer with a well-defined molecular weight distribution was obtained, whilst the ¹⁹F NMR showed the presence of the characteristic PFP fluorines, demonstrating that the PFP moiety did not undergo base catalyzed substitution reactions. Additionally, the MALDI-TOF-MS spectra shows a narrow molecular weight distribution, whereby the observed mass corresponds closely to the theoretical exact mass of the expected product with a sodium ion. Furthermore, the observed mass in MALDI-TOF-MS corresponds well to the average molecular weight calculated from ¹H NMR (FIG. 42-1).

### Selective Amine and Thiol Termination

As shown above, the PFP moiety displays a high reactivity towards thiols, amines and alkoxides. When the selective termination with thiols and amines was attempted for pentafluorobenzyl bromide initiated polymerization in MeCN, the high reactivity of the PFP moiety was shown to be problematic. In the case of amines, piperidine was chosen as a model compound, since it quickly terminates the polymerization due to its high nucleophilicity and low steric hindrance, whereas other amines have been reported to require considerably longer reaction times to achieve complete termination. Hence, we assessed the selectivity after incubating the polymerization with a 10-fold molar excess of piperidine for 24 hours as a representative example for less reactive amines. However, under these conditions it was noticed that more than 30% of the PFP moiety had already reacted with piperidine (FIG. 10).

Similarly, selective thiol termination also encountered problems, as experiments revealed that by cooling the reaction mixture, negligible conversion of the PFP-groups occurred within 3 hours at 0° C., and already by 4 hours noticeable conversion was observed in ¹⁹F NMR. However, upon isolation of the polymer after 3 hours, ¹H NMR revealed incomplete termination for the model thiol, tert-butyl N-(2-mercaptoethyl)carbamate. These results indicated that both the 2-oxazolinium and the PFP moieties possessed similar reactivity towards these nucleophiles under these conditions.

In order to promote selective reaction with the 2-oxazolinium species, we explored the use of the apolar chlorobenzene as a polymerization solvent, hypothesizing that the formation of the charged Meisenheimer intermediate in the para-fluoro substitution would be energetically unfavorable, whereas the 2-oxazolinium would increase in reactivity due to reduced solvation by the apolar chlorobenzene (PhCl). This hypothesis was experimentally confirmed by performing both the polymerization and the termination step chlorobenzene, whereby the isolated products for piperidine and tert-butyl N-(2-mercaptoethyl)carbamate termination yielded the desired products with the intact PFP moiety, as confirmed by the characterization data displayed in FIGS. 11-14. Please note that the molecular weight distribution observed in the DMF-GPC is slightly broader for the polymers prepared in PhCl than the polymers prepared in MeCN, which can be attributed to slow initiation by pentafluorobenzyl tosylate in PhCl. Furthermore, the MALDI-TOF-MS spectra observed in FIG. 13 shows the presence of different molecular ions, which can be attributed to the fragmentation of the boc-group under the MALDI-TOF-MS conditions. Boc-groups have been reported to be labile under MALDI-TOF-conditions, no matter the acidity of the matrix, trans-2-[3-(4-tert-butylphenyl)-2-methyl-2-propenylidene]malononitrile in this case, and give rise to populations closely resembling the potassium adduct, despite spiking the analyte solution with sodium trifluoroacetate. Although correct assignment of the MALDI-TOF-MS spectrum is not possible, the fit of simulated potassium adducts, as was reported on other polymers, points to the presence of a boc group, which was also confirmed by ¹H NMR.

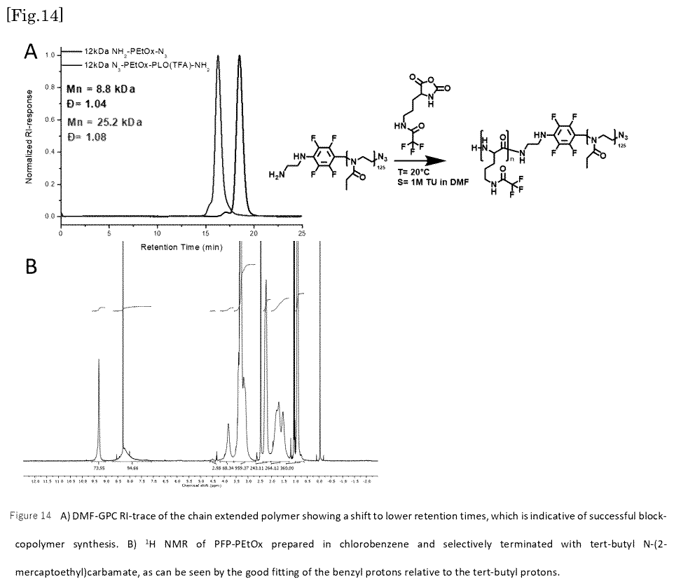

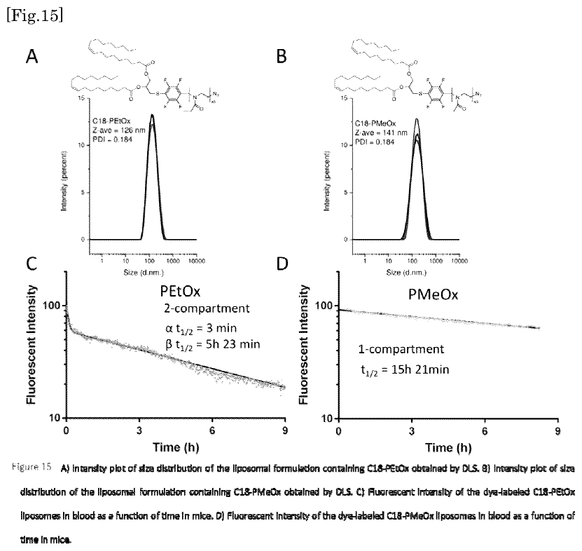

Heterotelechelic PFP-POx derivatives and their application in nanomedicine Next, we explored the developed methodology to synthesize heterotelechelic POx with relevant applications in nanomedicine. First, we synthesized an α-amine and ω-azide heterotelechelic POx through selective azide termination of the CROP and subsequent para-fluoro substitution with excess ethylenediamine. These polymers were subsequently utilized as macro-initiators for the ring-opening polymerization of N-carboxyanhydrides, thus yielding N₃-POx-polypeptide block-copolymers. These polymers would be well-suited for nanomedicine, since similar N₃-PEG-polypeptide block-copolymers have shown great promise in pre-clinical and clinical trials. For this purpose, the synthesis of POX-polypeptide block-copolymers was explored with trifluoroacetyl (TFA) L-onithine carboxyanhydride as a monomer, as this would yield after TFA-deprotection a polycationic block, which would be suitable for the condensation of polyanionic biomacromolecules such as DNA and RNA. The characterization data of the chain extension experiment is shown in FIG. 14. FIG. 14A shows that the chain extension experiment was successful, as the obtained block-copolymer displayed a narrow molecular weight distribution in GPC and no detectable macro-initiator remained, which indicates that the developed methodology is well-suited for this purpose. Furthermore, the well-controlled nature of the polymerization indicates that the tetrafluoroaniline group does not initiate the NCA polymerization, which was expected as it should be 10 orders of magnitude less nucleophilic than the primary amine. FIG. 14B confirms this, as the relative ratio of the protons of the respective blocks correspond well to the employed initiator:monomer ratio.

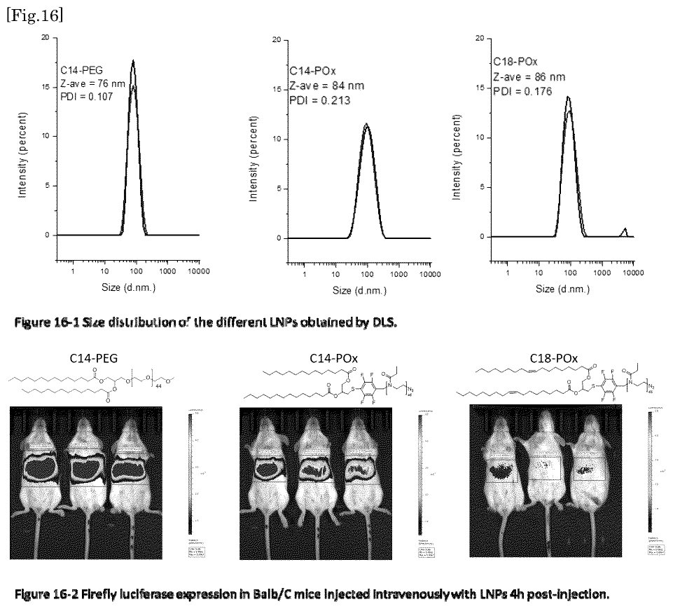

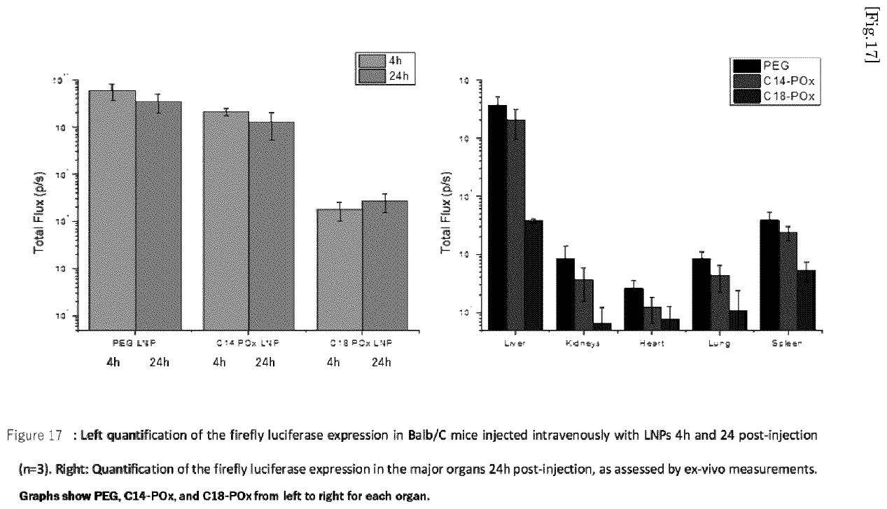

Besides synthesizing block-copolymers, we also explored the synthesis of lipid-polymer conjugates, utilizing the 1-thioglycerol modified PFP-POx-N₃ as a precursor. This precursor polymer could efficiently be utilized for the synthesis of a variety of lipid-conjugates through a simple Steglich esterification with the corresponding fatty acids. Three lipid conjugates were synthesized utilizing oleic acid (C18) and myristic acid (C14) as the lipid tail, yielding C18-PEtOx-N₃, C14-PEtOx-N₃ and C18-PMeOx-N₃ (characterization data of C14-PEtOx-N₃ is shown in FIGS. 42-2 as a representative example). These lipids were subsequently explored for the formulation of liposomes with 1,2-dioleoyl-sn-glycero-3-phosphocholine (DOPC)/Cholesterol/dioleate-POx in a 62.5/32.5/5 molar by a thin-film hydration method and subsequent extrusion of the obtained solution. Through this method, well-defined liposomes were obtained with a hydrodynamic radius of 126 nm and 141 nm for the C18-PEtOx and C18-PMeOx formulations, respectively (FIG. 16). Next, fluorescently labeled liposomes were prepared by incorporating the lipophilic dioctadecyl-3,3,3,3 tetramethylindodicarbocyanine dye (DiD) in the formulations. This was done to assess the effects of the tetrafluorophenyl (TFP) linker on the in vivo circulation of the obtained liposomes by intravital confocal laser scanning microscopy, since the presence of this hydrophobic linker might affect factors such as lipid-membrane fluidity or induce non-specific protein adsorption. However, the obtained blood circulation profiles of the lipids suggest that the TFP has a negligible influence on the pharmacokinetics of the liposome in mice, as the obtained half-lives corresponded well to earlier reported POx-liposomes that do not contain the TFP linker. Of note is that the earlier reported method relied on periodic blood sampling, whereby the relatively short α-phase of the C18-PEtOx could have been overlooked.

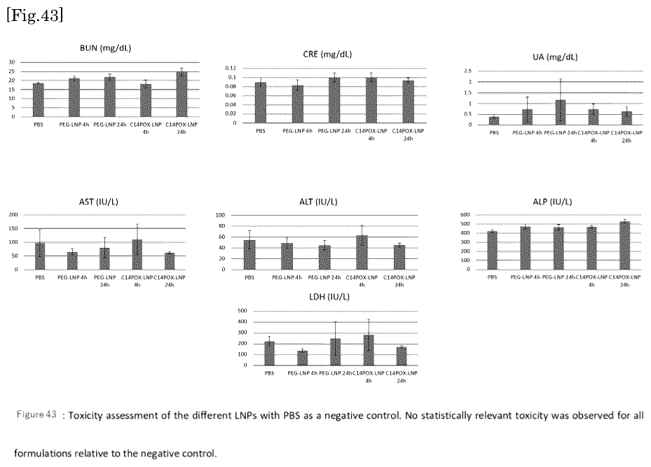

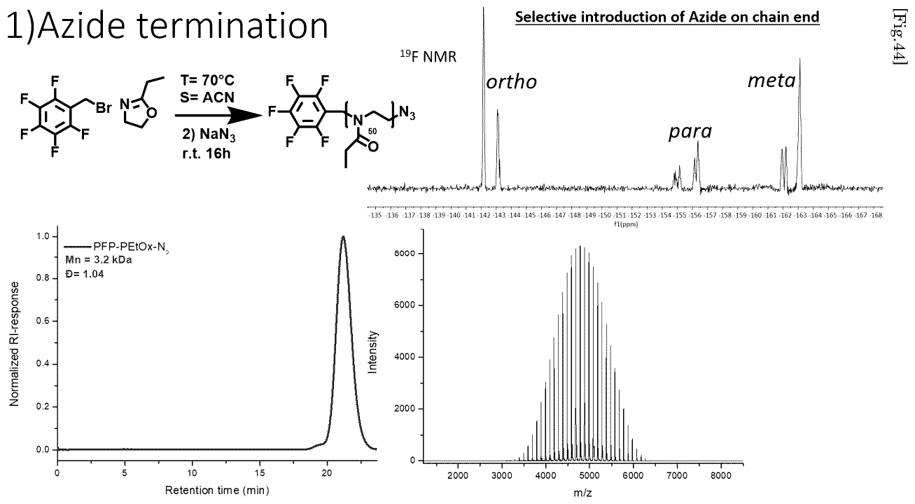

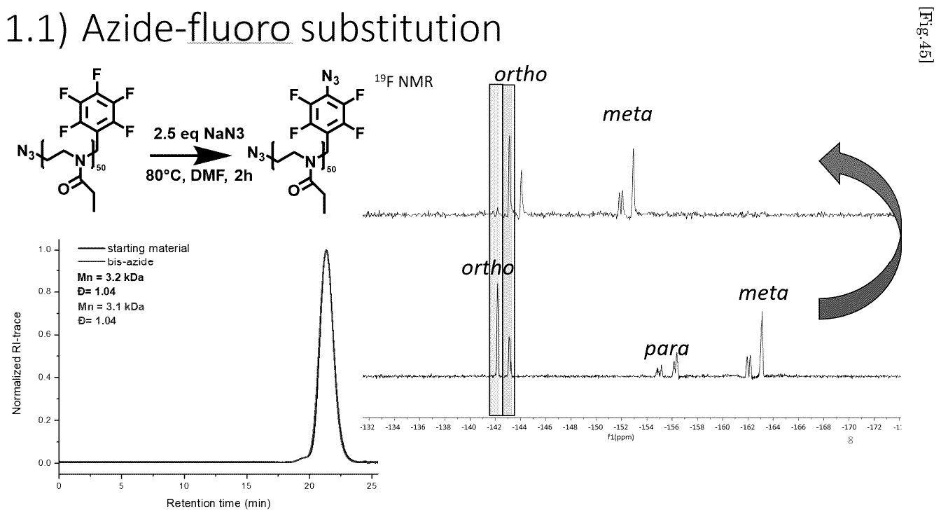

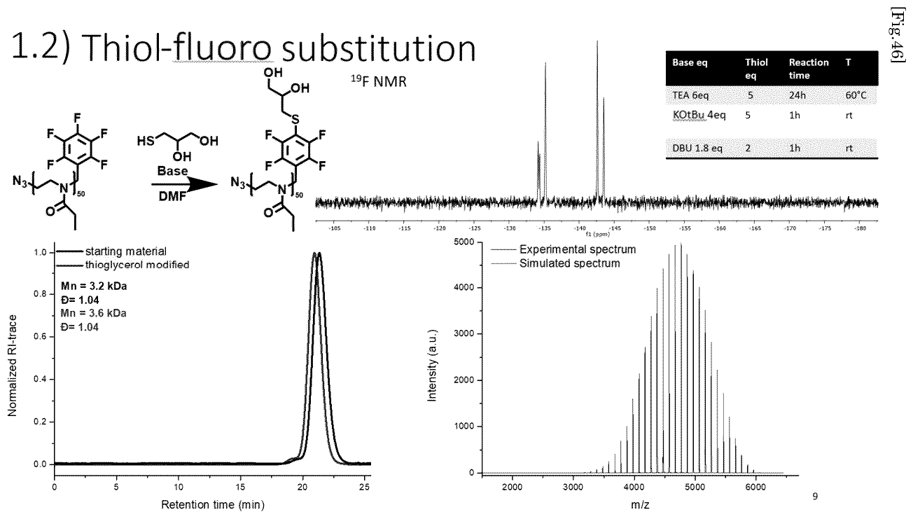

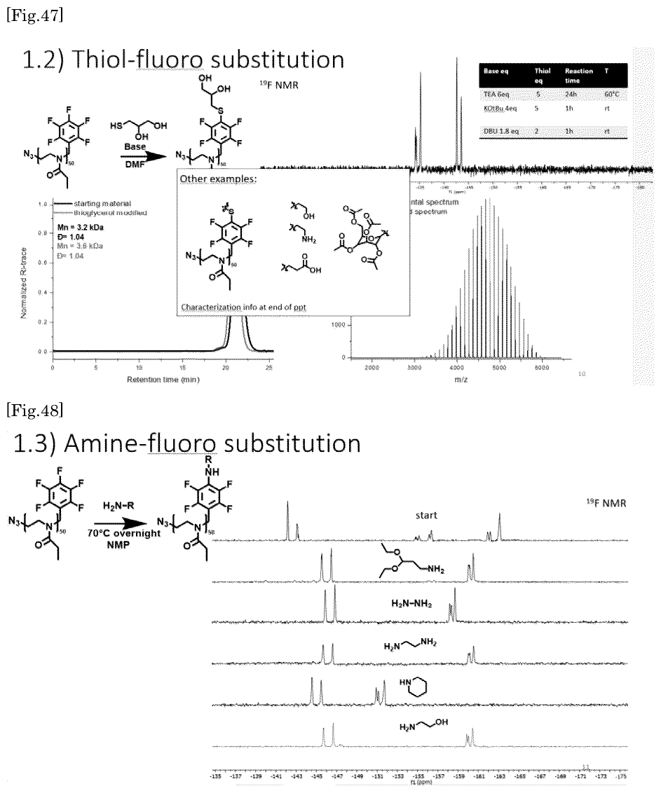

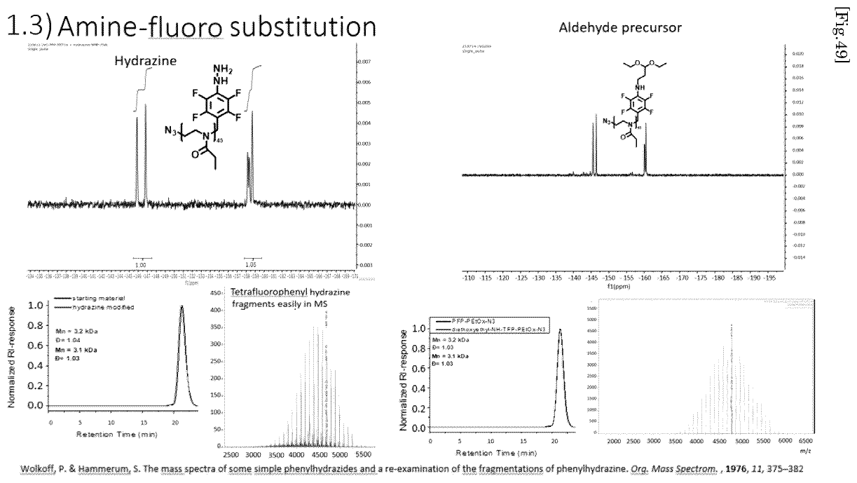

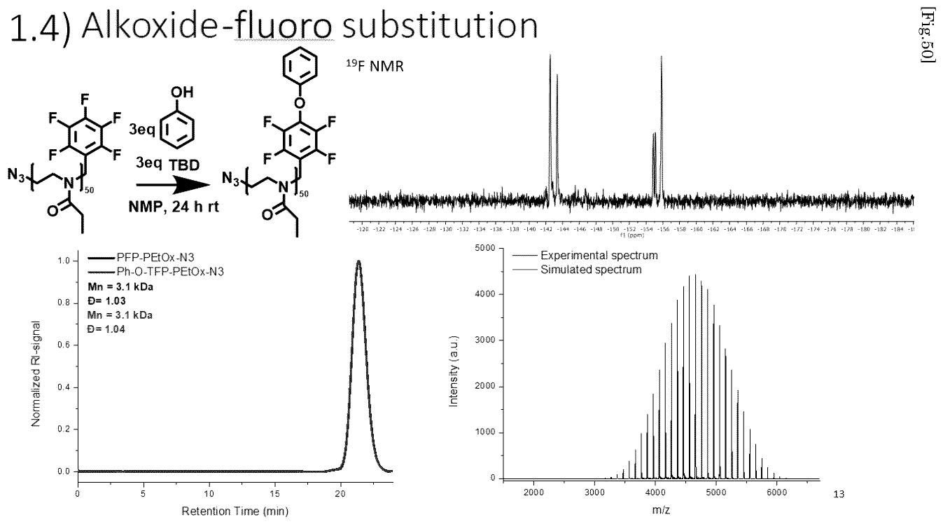

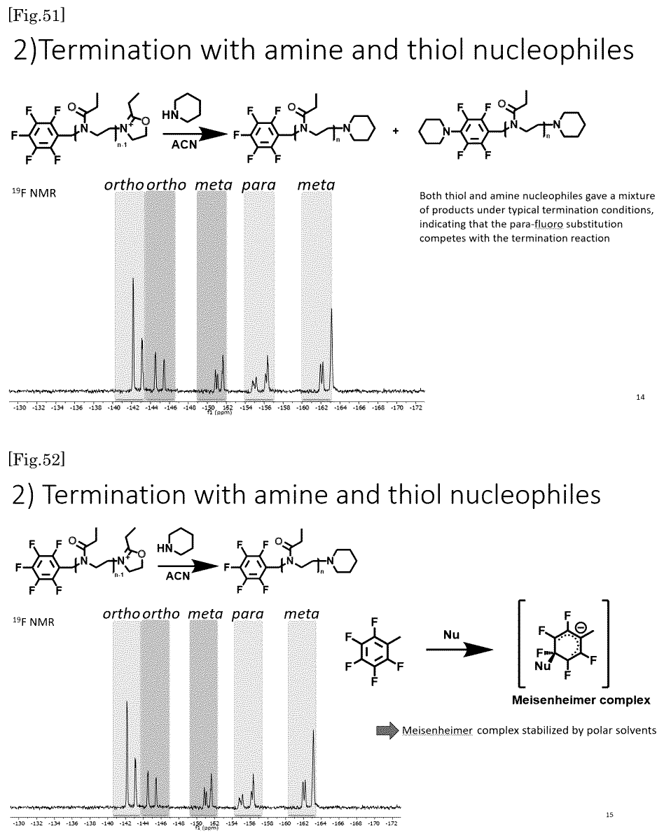

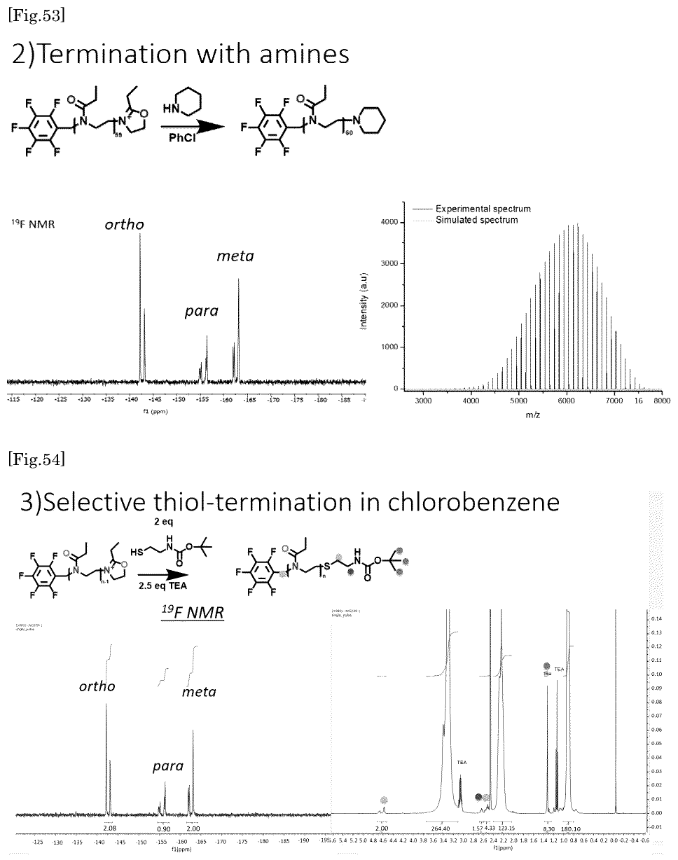

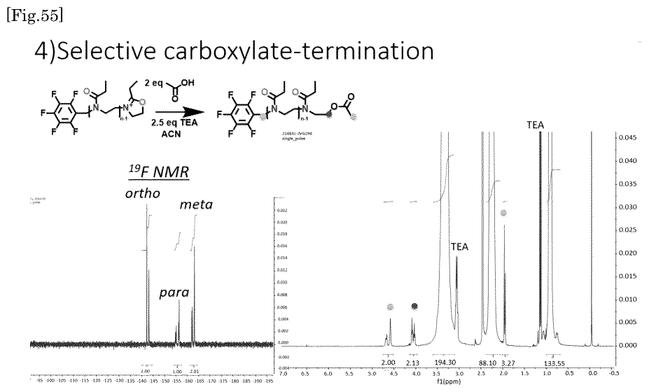

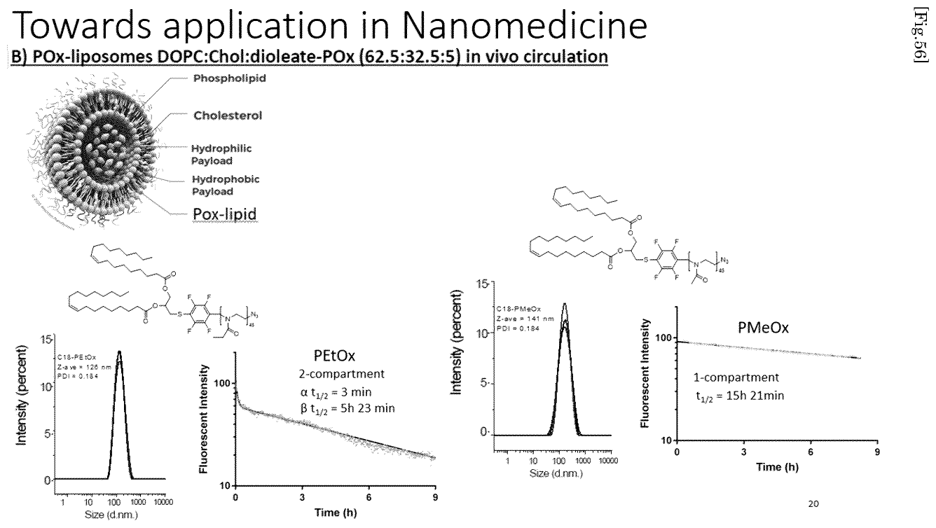

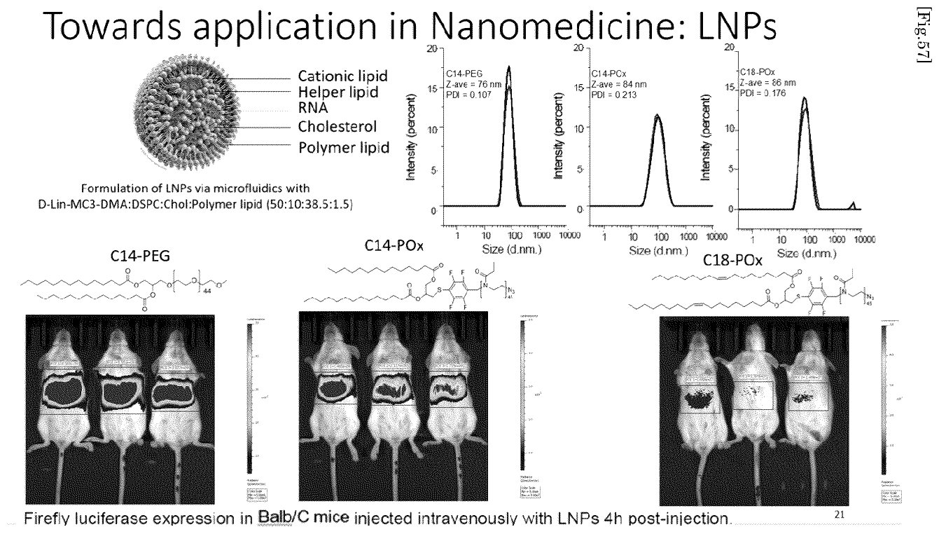

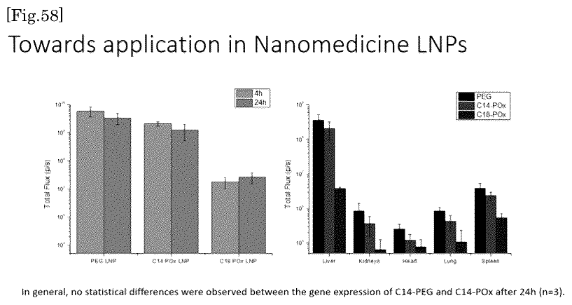

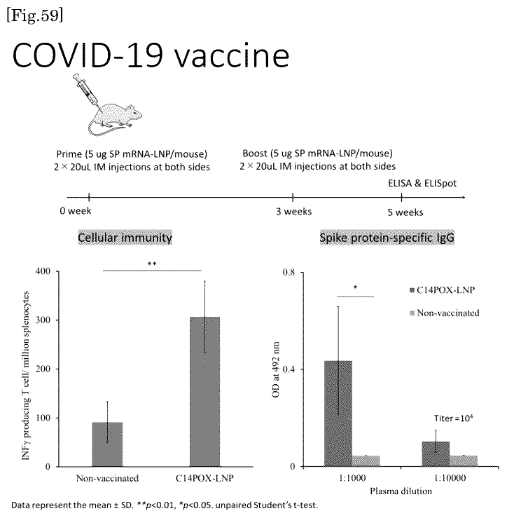

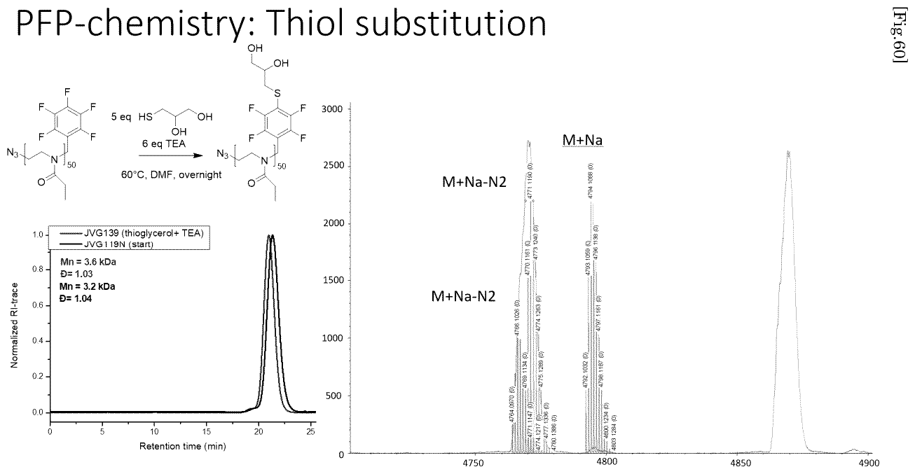

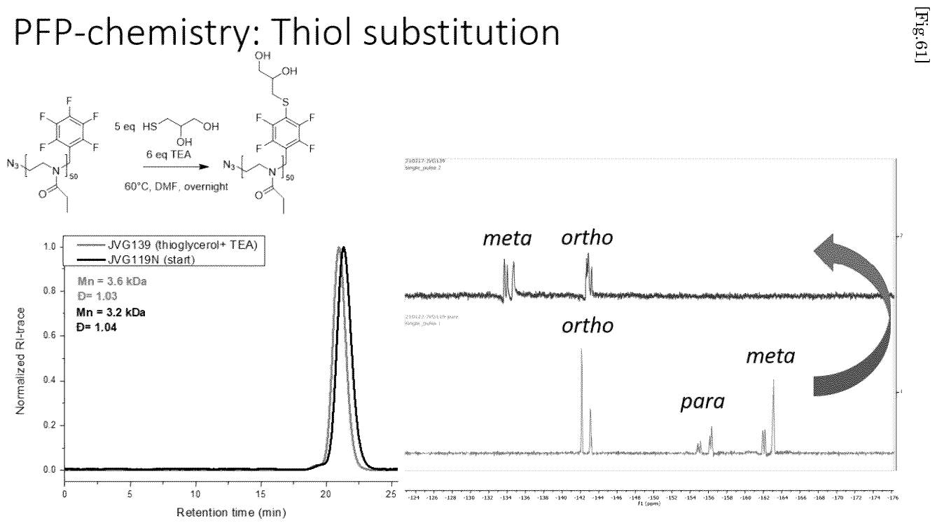

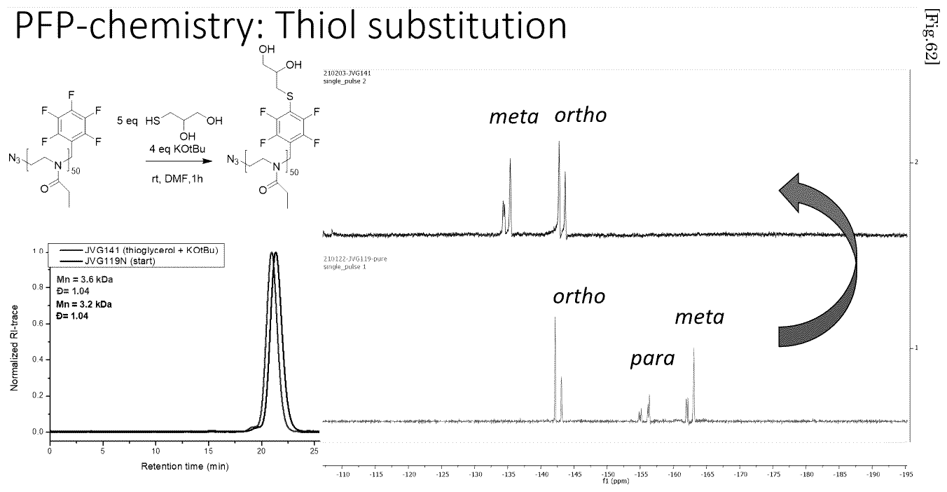

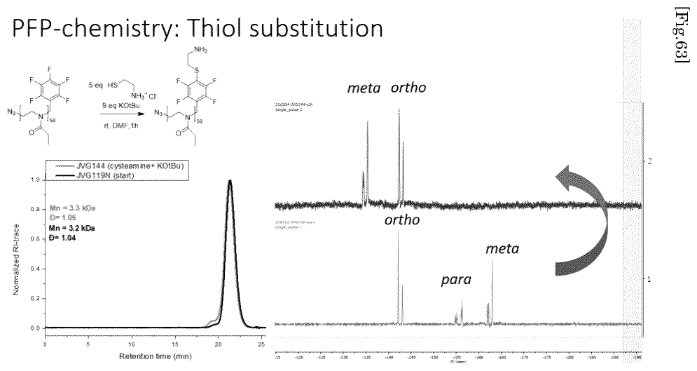

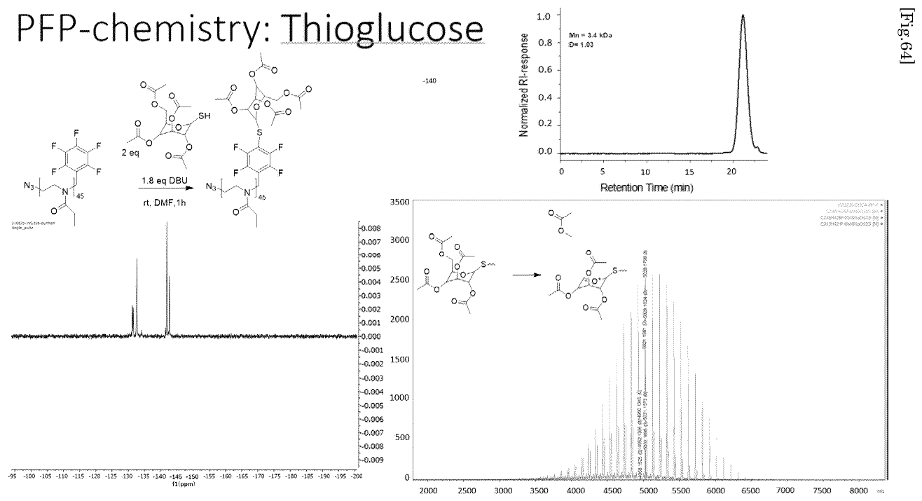

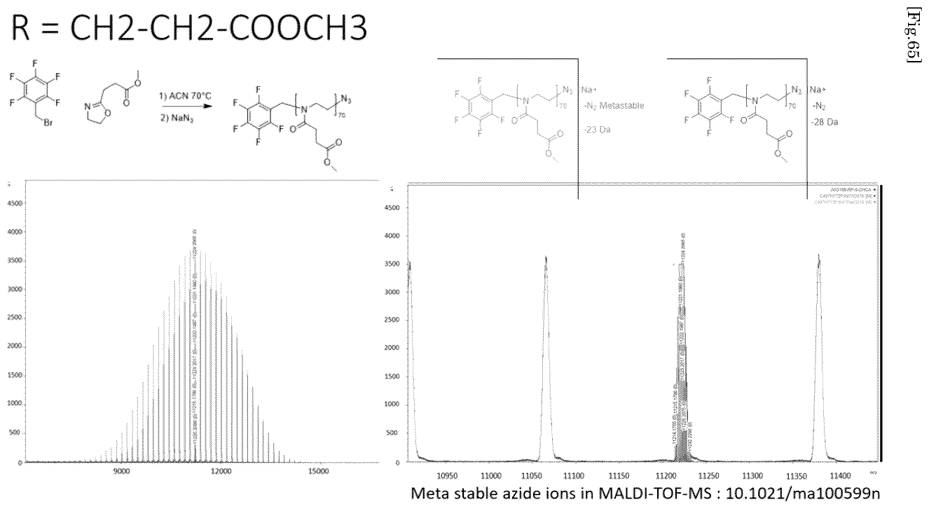

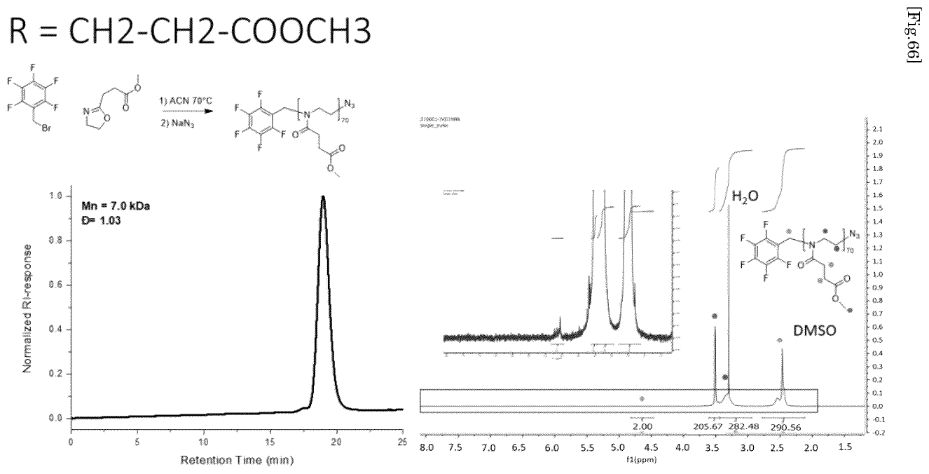

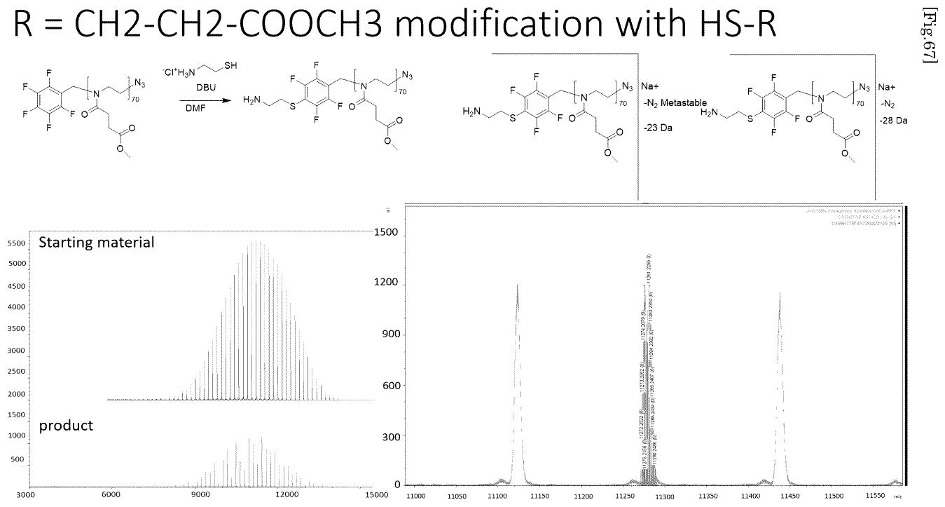

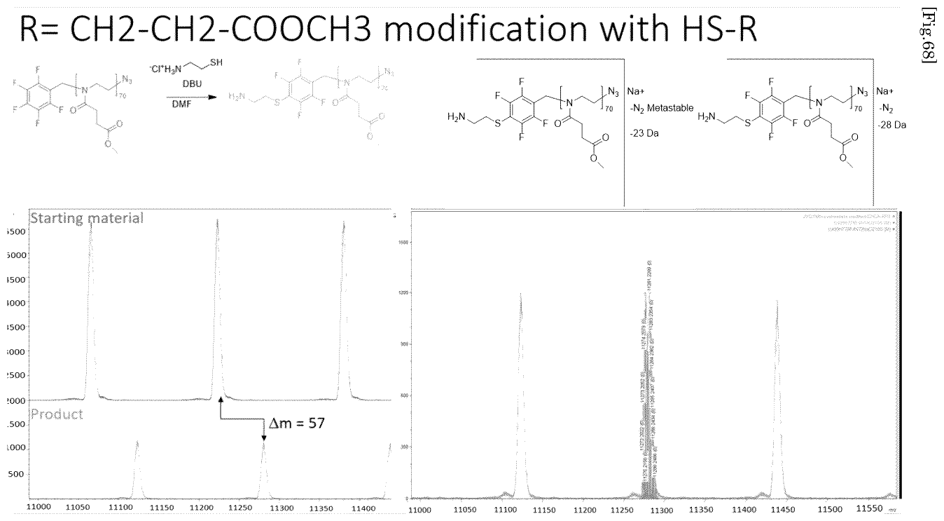

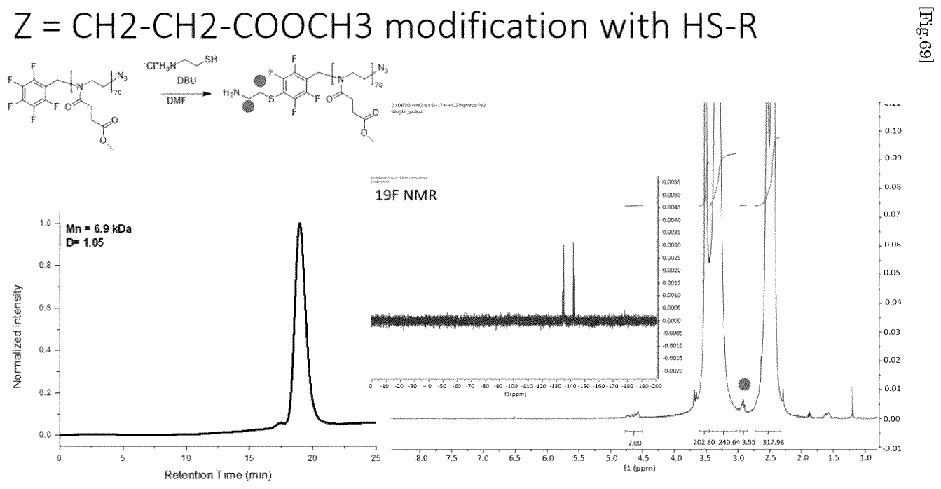

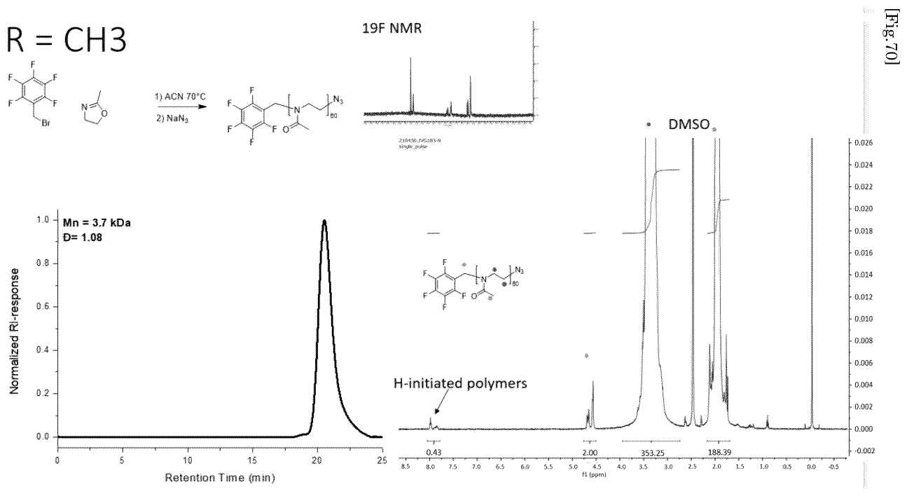

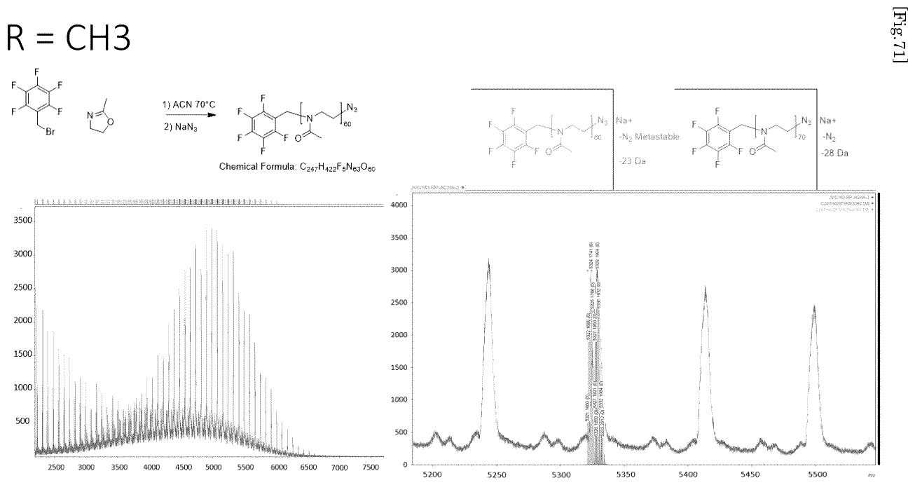

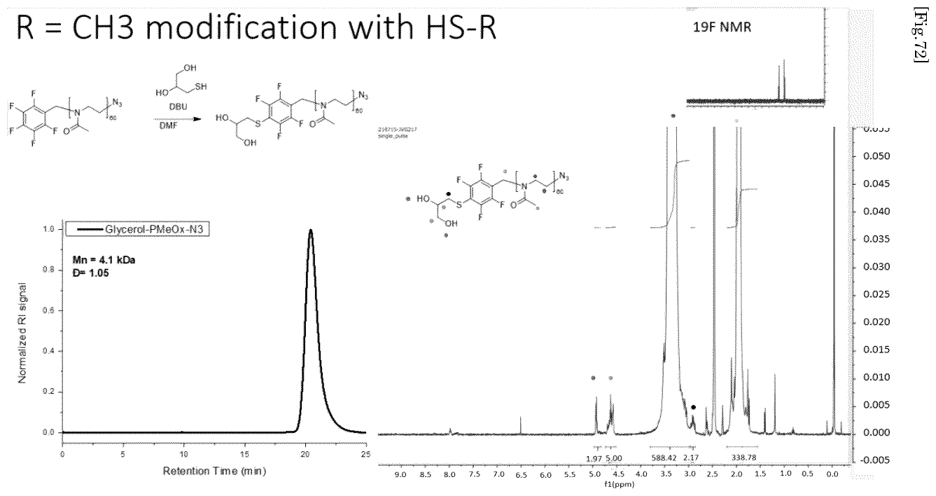

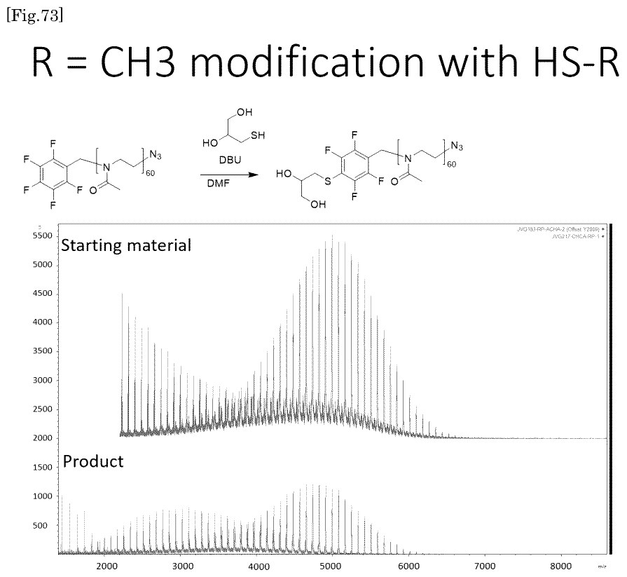

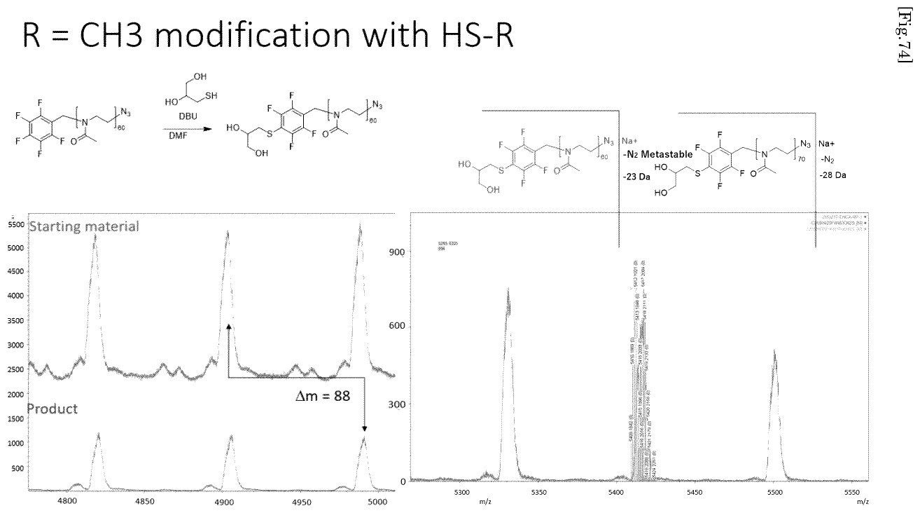

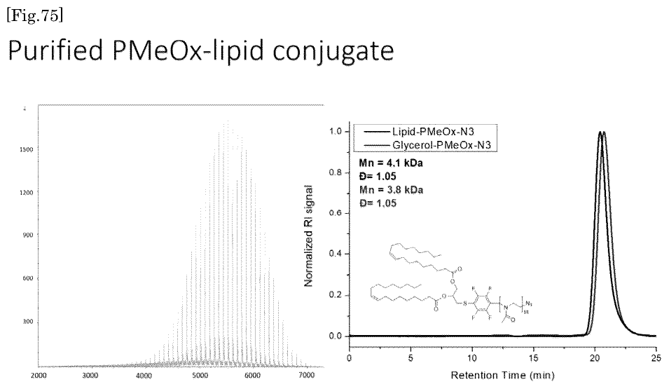

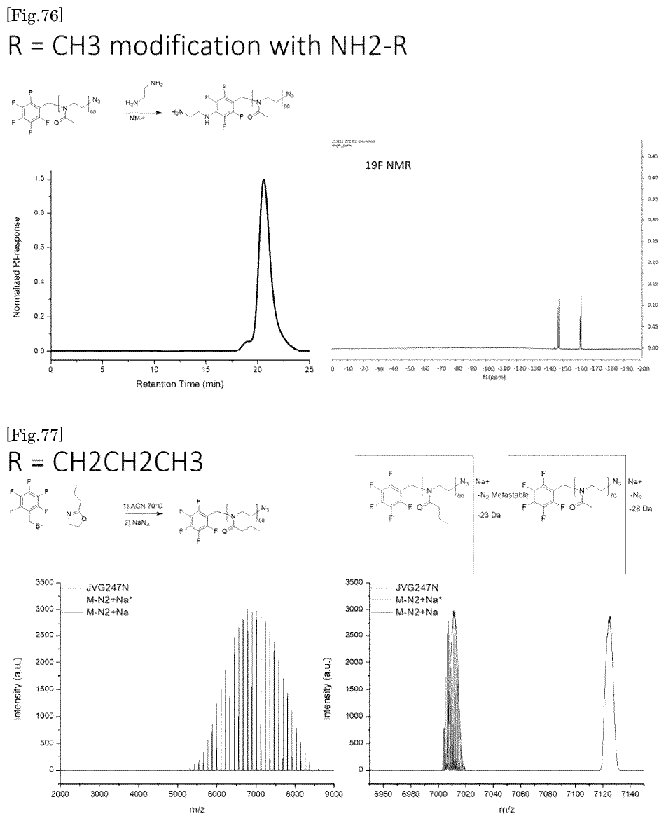

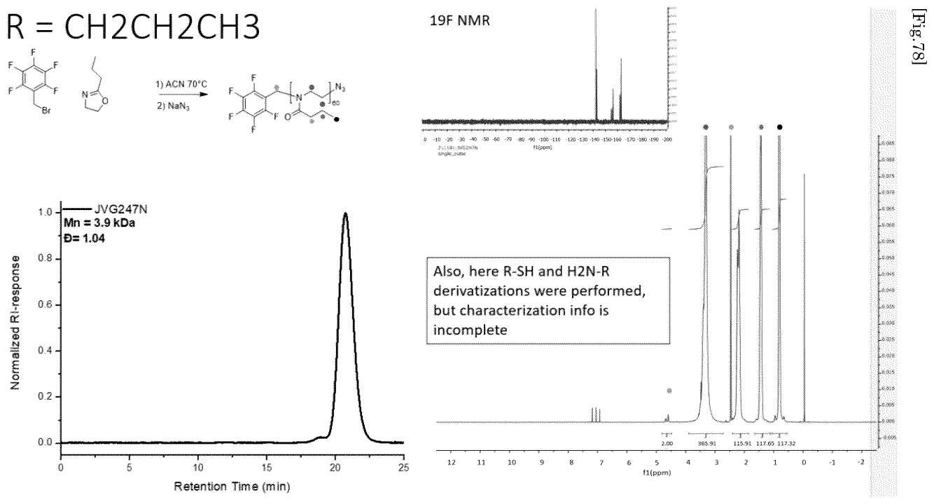

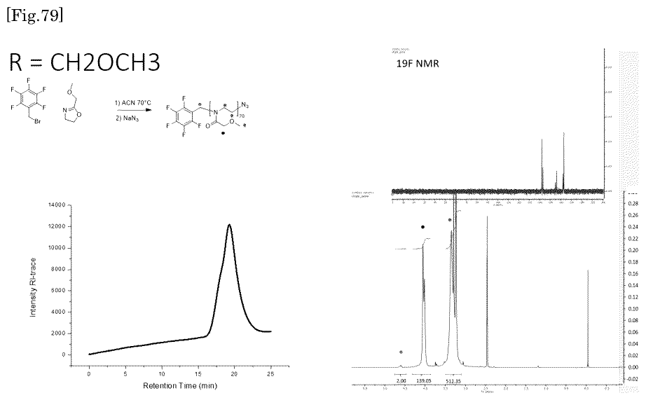

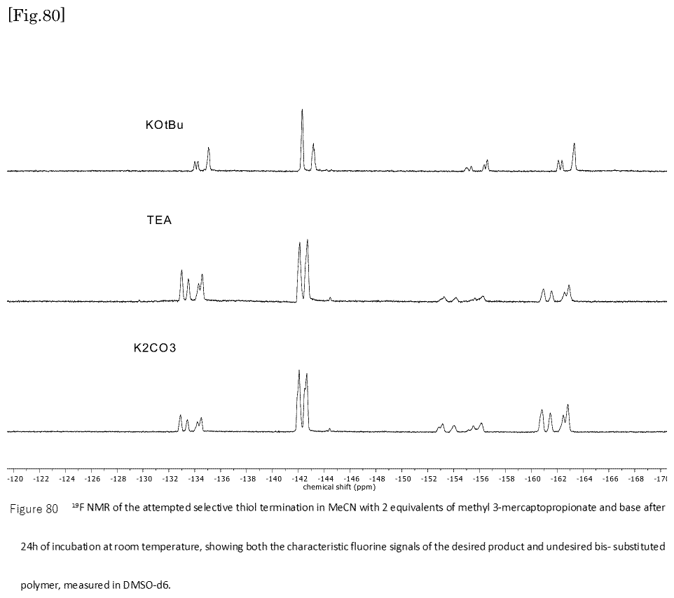

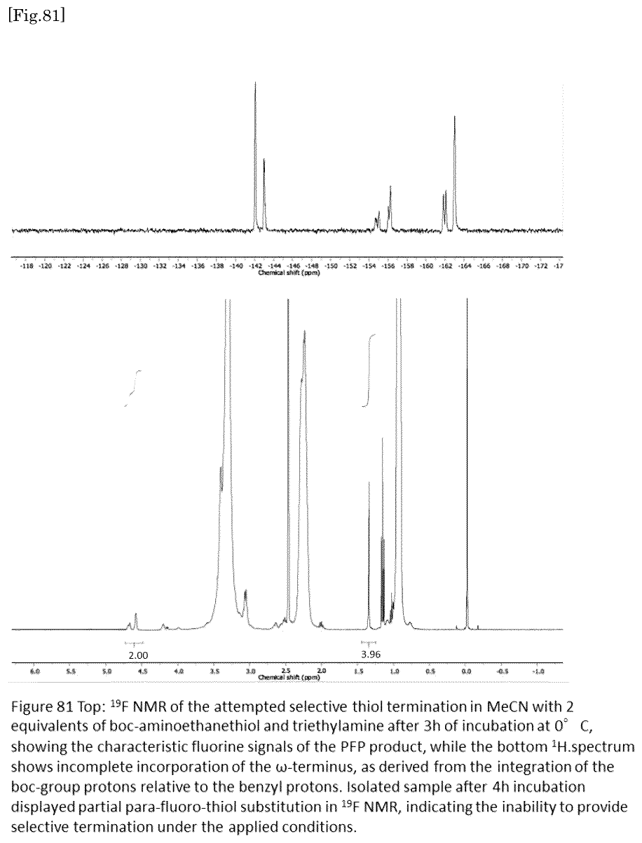

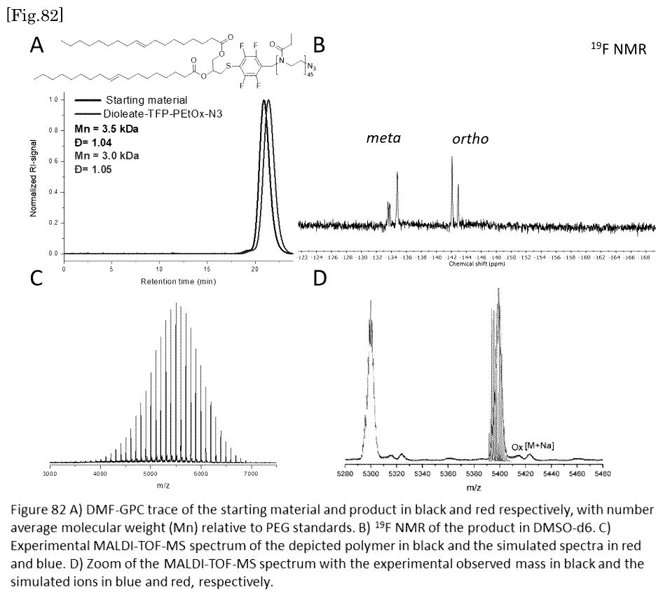

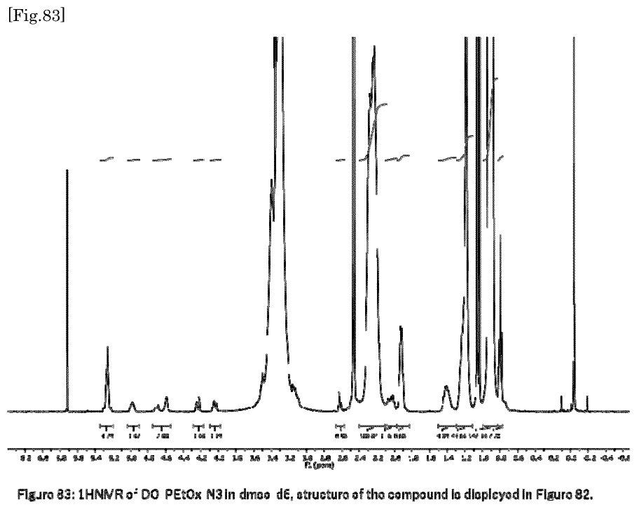

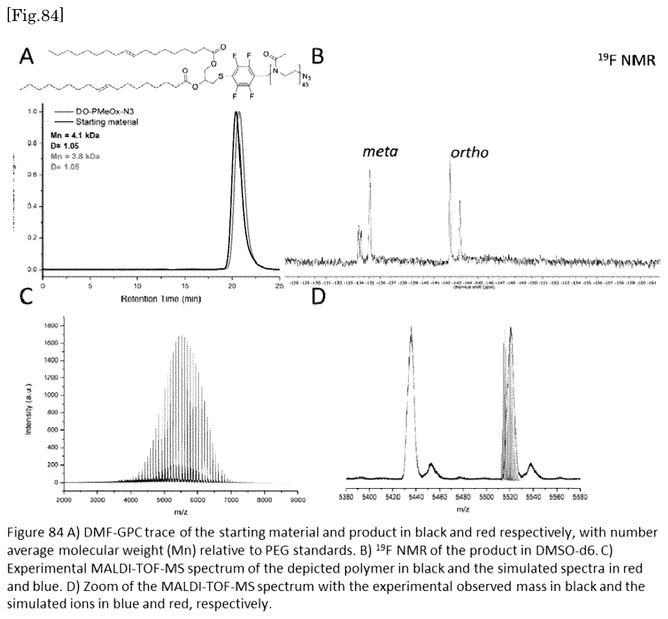

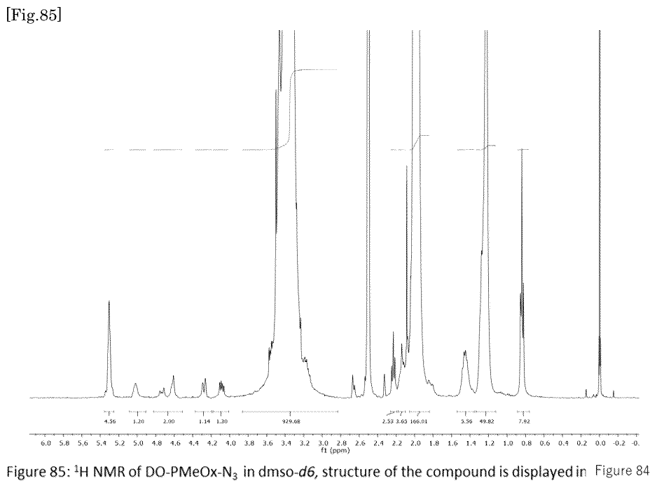

While the above results demonstrate that the TFP linker does not significantly affect the blood circulation of liposomes, the dynamic exchange of lipid formulations with lipoproteins in blood might be affected by the presence of a hydrophobic linker. In lipid nanoparticle (LNP) formulations with mRNA it has been reported that the length of the lipid tail of the PEG-lipid significantly affects the protein expression or knockdown of the encapsulated cargo. To assess the effect of the TFP linker, we prepared three LNP formulations, one containing DMG-PEG as the polymer lipid, a positive control capable of eliciting efficient hepatic protein expression, a C14-PEtOx and a C18-PEtOx, a negative control expected to provide little hepatic protein expression due to its limited desorption for the LNP. These lipids were used to encapsulate mRNA encoding for Firefly luciferase by hydrofluidic mixing, whereby the obtained LNPs displayed a narrow size-distribution in DLS (FIG. 16). Furthermore, all formulations displayed a comparable encapsulation efficiency ranging from 77-82% as assessed by a Ribogreen assay, indicating that the three investigated lipids could be utilized in a similar fashion for the formulation of mRNA. Next, the LNPs were intravenously injected in the tail vein of 5-week-old female Balb/C mice (n=3) and the protein expression was assessed 4 h and 24 h later by intraperitoneal injection of luciferin. After intraperitoneal administration of luciferin, the bioluminescence was measured by an in vivo imaging system, after which the mice were sacrificed and the protein expression was determined in the different organs ex vivo to measure the biodistribution (FIGS. 16-17). Overall, the synthesized C14-PEtOx lipid performed similarly to the positive control, which indicates that the TFP linker does not significantly affect the desorption of the polymer-lipid conjugate from the LNP. Similarly, the protein expression in the different organs was comparable to the positive control. This is in sharp contrast with the negative control, which showed negligible protein expression relative to the positive control, due to the inefficient desorption of the polymer-lipid conjugate, resulting in lower hepatic uptake and protein expression. In addition, all formulations displayed negligible toxicity, as assessed by measuring different toxicity markers in blood relative to mice injected with PBS (FIG. 43).

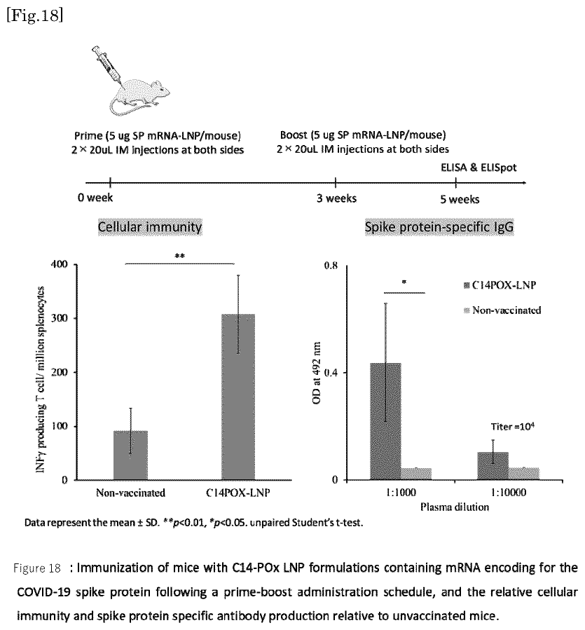

Since the newly developed POx-lipids displayed excellent potential for the delivery of mRNA, we assessed the potential of the C14-PEtOx formulation in the administration of mRNA encoding the COVID-19 spike protein, whereby the cellular immunity and antigen-specific antibody production was monitored. Here, mice were intramuscularly injected with 5 ug of mRNA in the tigh (prime), and three weeks later a second dose (boost) was administered. Two weeks after the last dose, blood was collected in heparinized tubes and mice were sacrificed to harvest the spleens for the quantification of humoral and cellular immunity, respectively. FIG. 18 shows that the C14-PEtOx formulation was able to induce both cellular immunity and antibody production relative to unvaccinated mice, demonstrating the potential of POx-lipids in LNP-vaccination technology.

### CONCLUSIONS

In conclusion, a modular synthetic approach was investigated to promote the facile diversification of POx α-termini. In this work we have shown that para-fluoro substitutions are well-suited to facilitate structural diversification of poly(2-oxazoline)s as a variety of O-, N-, S-nucleophiles were successfully introduced on the α-terminus. This was possible due to the careful tuning of the reaction conditions, thus first enabling selective reactions of the 2-oxazolinium species with O-, N-, and S-nucleophiles in the termination step of the CROP, and the subsequent derivatization of the pentafluorobenzyl group. The para-fluoro substitution therefore enables the synthesis of a wide range of telechelic POx in a single post-polymerization step. Next, we demonstrated the utility of this approach by synthesizing POx-lipid conjugates, and POx-based macro-initiators for NCA polymerization, thus yielding POx-polypeptide block-copolymers. Given the prominence of PEG-polypeptide systems in nanomedicine, the newly synthesized systems should ideally be suited for the development of drug delivery systems. Next, the in vivo circulation and in vivo mRNA expression was evaluated of POx-based liposomes and lipid nanoparticles, respectively. The circulation of the POx-based liposomes featuring the new tetrafluorobenzyl-linker corresponded well to earlier reports. In addition, mRNA expression of the POx-LNPs was comparable to commercial PEG-based formulations, and the formulations did not show any significant toxicological markers relative to PBS. These results suggest that the tetrafluorophenyl linker has little to no significant impact on the in vivo performance of POx-based lipid systems.

Precise control over the end-groups of biocompatible polymers has been proven key in enabling polymer-based therapeutics and nanomedicine, most notably in the form of PEGylation. However, the diversification of end-groups can be a synthetically exhaustive process, especially for polymers prepared via ionic polymerization mechanisms, due to the limited functional group tolerance associated with the polymerization mechanism. Within this contribution, a single step post-polymerization modification approach is presented for facile end-group diversification of poly(2-oxazoline)s (POx) with a wide array of nucleophiles. More specifically, the differential reactivity of a pentafluorophenyl chain-end and the living 2-oxazolinium chain-end was established via tuning of the reaction parameters, which enables the selective introduction of an array of nucleophiles via the termination of the CROP, and a subsequent nucleophilic para-fluoro substitution, thus facilitating end-group diversification of POx in a single post-polymerization modification step. This work therefore presents an attractive synthetic route to facilitate POxylation. The value of this approach is demonstrated through the synthesis of well-defined lipid-polymer conjugates and block-copolymers of POx and polypeptides, which are both well-suited for drug and gene-delivery. Finally, the application of a lipid-POx conjugate for the formulation and delivery of mRNA lipid nanoparticles was investigated, which underlines the value of POx as a biocompatible polymer platform.

Accurate control and modulation of polymer termini has proven to be key in the application of polymers in biomedicine, which is perhaps best exemplified by PEGylation, where the polymer termini are exploited for the synthesis of well-defined nano-assemblies and/or bioconjugation of proteins, nucleic acids, or targeting ligands. The conceptually simple conjugation of a non-immunogenic, biocompatible (i.e. “stealth”) polymer is therefore a widespread and attractive approach to modulate the biodistribution and circulation of therapeutics. Another class of polymers well-suited for this purpose are poly(2-oxazoline)s and their structural relatives, i.e. poly(cyclic imino ether)s, which feature a high degree of structural versatility by merit of the variable ring-size and substituents of the cyclic imino-ether monomers. In addition to monomer design, a plethora of efficient post-polymerization modification chemistries are available to facilitate further tailoring of the polymer structure to the needs of specific applications, such as tissue engineering and drug/protein/gene delivery. Although the structural variation along the polymer backbone presents ample opportunities in biomedicine, facile end-group diversification remains a key feature in bioconjugation and the synthesis of advanced drug delivery systems.

In this respect, considerable efforts have demonstrated that a plethora of functionalities can be introduced on POx termini, although the process of end-group diversification is typically synthetically exhaustive. This can be mainly attributed to the challenging diversification of the α-terminus. Typically, functional moieties featuring the preferred tosylate or triflate leaving groups have a rather limited commercial availability, presumably due to their poor shelf-life, and therefore need to be synthesized and thoroughly purified in-house. Also, the steric and electronic factors of the initiators play a crucial role in facilitating fast initiation of the polymerization reaction. Due to these factors, every iteration of the α-terminus is synthetically exhaustive, as accurate studies of the polymerization kinetics for different 2-oxazoline monomers need to be performed, in addition to end-group fidelity assessments. Due to these limitations, a strategy that focusses on the post-polymerization diversification of a single end-group is synthetically more attractive, enabling divergent, yet highly reproducible synthesis. So far, only a handful of functional groups (azides, alkynes, esters, alkenes) can be directly introduced on the α-terminus, i.e. without protecting groups, to facilitate end-group diversification via post-polymerization modification. These functional groups are, however, poorly suited for the introduction of relatively simple molecules with diverse functionalities. While highly efficient, azide-alkyne cycloadditions are not synthetically attractive for this purpose, as this chemistry is better suited for bioconjugation Ester groups are relative unreactive, typically requiring harsh conditions or long reaction times for their derivatization which is not ideal. Lastly, alkenes could be modified by thermally or photo-initiated thiol-ene chemistry, however, the radical nature of this process is not ideal for end-group diversification of POx, as intrinsic chain transfer and termination reactions result in impurities and this reaction requires substantial excess of the thiol. Other functionalities that require the use of protecting groups (e.g. aldehydes, ketones, carboxylates, amines, alcohols, or maleimides), require a deprotection step prior to subsequent reactions, whereby incomplete deprotection and/or incomplete reaction compromises the purity of the heterotelechelic polymers, thus requiring additional purification. Finally, the abovementioned substrates have a relatively narrow substrate scope and the determination of quantitative conversion for each synthetic step can be challenging.

This difficult end-group diversification of the α-terminus is contrasted by the facile end-group diversification of the α-terminus, whereby the electrophilic 2-oxazolinium can be ring-opened with a wide variety of commercially available O-, N-, or S-based nucleophiles. Inspired by the simplicity and broad scope of the nucleophilic termination reaction, we sought to initiate the cationic ring-opening polymerization with an electrophilic moiety, susceptible to nucleophilic substitution with a similar substrate scope. In this work, we demonstrate that differential reactivity between the living 2-oxazolinium α-terminus and an electrophilic pentafluorophenyl α-terminus can be established for a variety of nucleophiles via tuning of the reaction parameters. The intact pentafluorophenyl moiety underwent a selective para-fluor substitution with O-, N-, and S-nucleophiles, thus enabling facile end-group diversification. The modification of this moiety is in principle orthogonal with several functional groups that have been incorporated along the polymer chain, which we demonstrated for ester functional POx. Hence this approach is valuable for the synthesis of drug and gene-delivery vehicles, which is demonstrated through the synthesis of well-defined lipid-POx conjugates, as well as POx-polypeptide block-copolymers. Finally, we report the first application of a lipid-POx conjugate for the formulation and delivery of mRNA lipid nanoparticles, which underlines the value of POx as a viable candidate for gene-delivery applications.

### INDUSTRIAL AVAILABILITY

The polymers of the invention can be used in medical materials, e.g. Vaccines, drug delivery formulations, gene delivery formulations, protein conjugates, non-active pharmaceutical ingredients, excipients, surface coatings.

1. A compound of formula (I) 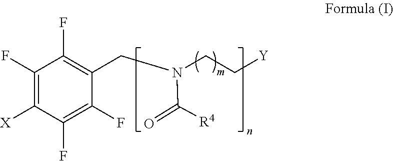
wherein X is halogen, —SR², —OR², —CN, —NHR², —NR² R³, —NHNHR², —N═NR², —N₃, —C≡C—R², —NHOR², —ONR²R³, heteroaryl which may be substituted, a group which can form lipid based nano-particle (e.g., a formulation containing a lipid nano-particle (LNP), liposome, or lipoplex) or micelle including polycationic group, polyanionic group, or lipid soluble groups such as groups derived from fatty acid, preferably F, —SR², —OR², —CN, —NHR², —N₃, 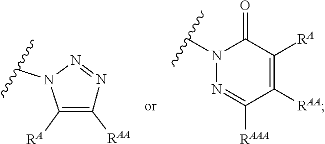
Y is SR², —OR² (provided that when X is F, it is not —OH), —CN, —NHR², —NR² R³, —NHNHR², —N═NR², —N₃, or heteroaryl which may be substituted, preferably —SR², —OR² (provided that when X is F, it is not —OH), —NHR², —NR² R³, —N₃, or 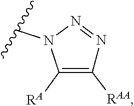
more preferably —SR², —OR² (provided that when X is F, it is not —OH), —NHR², or 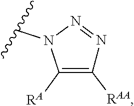
wherein repeating unit 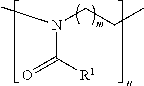
is independent of each occurrence and may be identical or different;
R¹ is selected from the group consisting of alkyl group having 1-40 carbon atoms which may be substituted, alkenyl group having 2-40 carbon atoms which may be substituted, alkynyl group having 2-40 carbon atoms which may be substituted, aryl which may be substituted, heteroaryl which may be substituted;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, sugar derivatives, and repeating unit or in the case of —NR² R³, R² and R³ may be linked together to form heterocyclyl which may be substituted, preferably —CH₂CH(OR^{C})CH₂OR^{C},
R^{A}, R^{AA} and R^{AAA} are, independent of each other, selected from the group consisting of hydrogen, alkyl group having 1-20 carbon atoms which may be substituted, alkenyl group having 2-20 carbon atoms which may be substituted, and alkynyl group having 2-20 carbon atoms which may be substituted, or R^{A} and R^{AA} may be linked together to form ring, such as carbocycle, heterocycle, aryl, heterocyclyl, lactone, or lactam, and R^{A}, R^{AA} and/or R^{AAA} may be further linked to functional molecules such as labels, or biofunctional molecules including proteins, nucleic acid, etc.;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, lipid soluble groups such as alkyl group having 1-20 carbon atoms which may be substituted, alkenyl group having 2-20 carbon atoms which may be substituted, and alkynyl group having 2-20 carbon atoms which may be substituted, and oxygen protective groups including acyl groups such as acetyl, ether groups such as methoxymethyl etc.;
said alkyl, alkenyl and alkynyl is straight chain, branched chain or cyclic chain;
m is integer from 1 to 3;
and
n is integer from 1 to 2000,
or a pharmaceutically acceptable salt thereof.

2. The compound according to claim 1,
wherein X is halogen, —SR², —OR², —NHR², —NR² R³, —NHNHR², —N═NR², —N₃, —NHOR², —ONR²R³, heteroaryl which may be substituted,
Y is SR², —OR², —NHR², —NR² R³, —NHNHR², —N═NR², —N₃, or 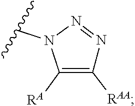
more preferably —SR², —OR², —NHR², or 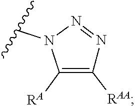
wherein
R¹ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, sugar derivatives, and repeating unit, or in the case of —NR² R³, R² and R³ may be linked together to form heterocyclyl which may be substituted, preferably —CH₂CH(OR^{C})CH₂OR^{C},
R^{A}, and R^{AA} are, independent of each other, selected from the group consisting of hydrogen, alkyl group having 1-20 carbon atoms which may be substituted, alkenyl group having 2-20 carbon atoms which may be substituted, and alkynyl group having 2-20 carbon atoms which may be substituted, or R^{A} and R^{AA} may be linked together to form carbocycle, heterocycle, aryl, heterocyclyl, lactone, or lactam, and R^{A} and/or R^{AA} may be further linked to labels, or biofunctional molecules including proteins, nucleic acid, etc.;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, lipid soluble groups such as alkyl group having 1-20 carbon atoms which may be substituted, alkenyl group having 2-20 carbon atoms which may be substituted, and alkynyl group having 2-20 carbon atoms which may be substituted, and oxygen protective groups;
said alkyl, alkenyl and alkynyl is straight chain, or branched chain;
substituent(s) of alkyl, alkenyl and alkynyl is (are), independent of each other, and also not limited to, but ester group, amino group, azide group, and
n is integer from 2 to 1000,
or a pharmaceutically acceptable salt thereof.

3. The compound according to claim 1,
wherein X is F, —SR², —OR², —NHR², —N₃, or 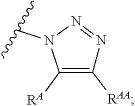
Y is —SR², —OR², —NHR², —N₃, or 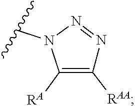
wherein
R¹ is selected from the group consisting of methyl, ethyl, propyl, butyl, —CH₂CH₂COOCH₃, —CH₂CH₂CH₂COOCH₃, —CH₂CH₂CH₂CH₂COOCH₃, butenyl, butynyl, or —CH₂OCH₃;
R² is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}—CH₂CH(OR^{C})CH₂OR^{C}, sugar derivatives, and the repeating unit,
R^{A} and R^{AA} are, independent of each other, selected from the group consisting of hydrogen, alkyl group having 1-20 carbon atoms which may be substituted, alkenyl group having 2-20 carbon atoms which may be substituted, and alkynyl group having 2-20 carbon atoms which may be substituted, or R^{A} and R^{AA} may be linked together to form carbocycle, heterocycle, aryl, heterocyclyl, lactone, or lactam, and R^{A} and/or R^{AA} may be further linked to functional molecules such as labels, or biofunctional molecules including proteins, nucleic acid, etc.;
R^{B} is selected from the group consisting of alkyl group having 1-6 carbon atoms which may be substituted;
R^{C} is selected from the group consisting of hydrogen, alkyl group having 1-20 carbon atoms which may be substituted, and alkenyl group having 2-20 carbon atoms which may be substituted;
said alkyl, and alkenyl are straight chain; and
n is an integer from 2 to 2000,
or a pharmaceutically acceptable salt thereof.

4. The compound according to claim 1,
wherein Y is 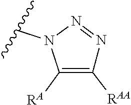
and R^{A} and/or R^{AA} may be linked to functional molecule selecting from the groups consisting of labels, or biofunctional molecules including proteins, and nucleic acid.

5. The compound according to claim 1,
wherein X is —OR² or —SR²,
Y is 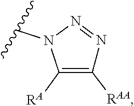
R² is —CH₂CH(OR^{C})CH₂OR^{C}, and
R^{C} is selected from the group consisting of hydrogen, alkyl group having 1-20 carbon atoms which may be substituted, and alkenyl group having 2-20 carbon atoms which may be substituted.

6. The compound according to claim 1,
wherein X is —OR² or —SR²,
R² is —CH₂CH(OR^{C})CH₂OR^{C}, and
R^{C} is selected from the group consisting of alkyl group having 1-20 carbon atoms which may be substituted, and alkenyl group having 2-20 carbon atoms which may be substituted.

7. The compound according to claim 1,
wherein X is —OR² SR², or —NHR²
R² is —CH₂CH(OR^{C})CH₂OR^{C}, and
R^{C} is H.

8. The compound according to claim 1,
wherein X is —OR², —SR², or —NHR²
R² is —CH₂CH(OR^{C})CH₂OR^{C}, and
R^{C} is oxygen protective group.

9. The compound according to claim 1, wherein X is a group which can form lipid based nano-particle (e.g., a formulation containing a lipid nano-particle (LNP), liposome, or lipoplex) liposome or micelle.

10. The compound according to claim 1, wherein sugar derivative is glucose derivative.

11. A composition comprising the compound according to claim 1.

12. A composition comprising the compound according to claim 1, wherein the composition is vaccine.

13. A composition comprising the compound according to claim 1, wherein the composition is used for the diagnostic probes.

14. A compound of formula (II) 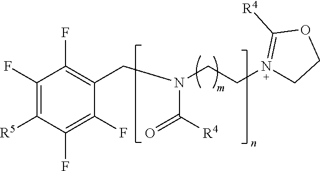
wherein repeating unit 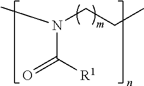
is independent of each occurrence and may be identical or different;
R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R⁵ is selected from the group consisting of —SR², —OR², —CN, —NHR², —NR² R³, —NHNHR², —N═NR², and —N₃, —NHOR², -and —ONR²R³;
R² and R³ are, independent of each other, selected from the group consisting of hydrogen, alkyl group having 1-6 carbon atoms which may be substituted, alkenyl group having 2-6 carbon atoms which may be substituted, alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case that R⁵ is —NR² R³, R² and R³ may be linked together to form heterocyclyl which may be substituted, preferably —CH₂CH(OR^{C})CH₂OR^{C};
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, alkyl group having 1-20 carbon atoms which may be substituted, or alkenyl group having 2-20 carbon atoms which may be substituted;
said alkyl, and alkenyl are straight chain;
m is integer from 1 to 3;
and
n is integer from 2 to 2000.

15. The compound according to claim 14,
R⁵ is —OR², —SR², or —NHR²,
R² is —CH₂CH(OR^{C})CH₂OR^{C}, and
R^{C} is selected from the group consisting of alkyl group having 1-20 carbon atoms which may be substituted, or alkenyl group having 2-20 carbon atoms which may be substituted.

16. The compound according to claim 14,
R⁵ is —OR², —SR², or —NHR²
R² is —CH₂CH(OR^{C})CH₂OR^{C}, and
R^{C} is H.

17. The compound according to claim 14,
R⁵ is —OR² or —SR², or —NHR²,
R² is —CH₂CH(OR^{C})CH₂OR^{C}, and
R^{C} is oxygen protective group.

18. A method for producing a compound of Formula (III), 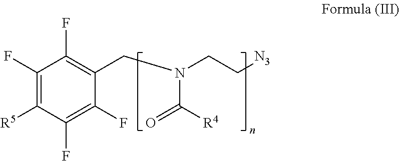
wherein repeating unit is independent of each occurrence and may be identical or different;
R⁴ is selected from the group consisting of alkyl group having 1-6 carbon atoms which may be substituted, alkenyl group having 2-6 carbon atoms which may be substituted, alkynyl group having 2-6 carbon atoms which may be substituted, aryl which may be substituted, heteroaryl which may be substituted;
R⁵ is selected from the group consisting of halogen, —SR², —OR², —CN, —NHR², —NR² R³, —NHNHR², —N═NR², —N₃, —NHOR², -and —ONR²R³;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, alkyl group having 1-20 carbon atoms which may be substituted, or alkenyl group having 2-20 carbon atoms which may be substituted;
said alkyl, and alkenyl are straight chain; and
n is integer from 2 to 2000,
comprising the following steps;
(a) a compound of Formula (IV) 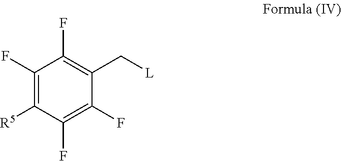
wherein L is a leaving group;
is reacted with a compound of Formula (V) 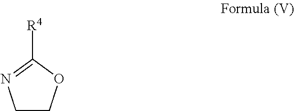
wherein R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
in the presence of base to obtain a compound of Formula (II) 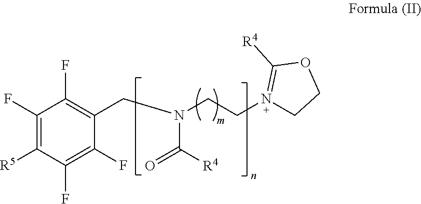
(b) a compound of Formula (II) is reacted with an azide reagent to obtain a compound of Formula (III).

19. The method for producing the compound of formula (III) according to claim 18, wherein the leaving group is selected from the group consisting of halogen, OTf, ONs, and OTs.

20. The method for producing the compound of formula (III) according to claim 18, wherein the base is selected from the group consisting of Et₃N, DBU, NMP, TBD, KOH, iPr₂NEt, and t-BuOK.

21. The method for producing the compound of formula (III) according to claim 18, wherein the azide reagent is selected from the group consisting of NaN₃, TMSN₃, TsN₃, tetrabutylammonium azide, and diphenyl phosphoryl azide.

22. A method for producing the compound of formula (III), 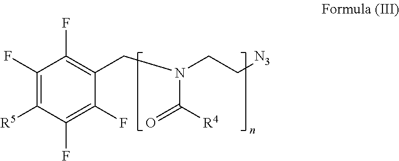
wherein repeating unit is independent of each occurrence and may be identical or different;
R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R⁵ is selected from the group consisting of halogen, —SR², —OR², —CN, —NHR², —NR² R³, —NHNHR², —N═NR², and —N₃, —NHOR², —ONR²R³;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, alkyl group having 1-20 carbon atoms which may be substituted, or alkenyl group having 2-20 carbon atoms which may be substituted;
said alkyl, and alkenyl are straight chain; and
n is integer from 2 to 2000,
comprising the following steps;
(a) a compound of Formula (IIIa) 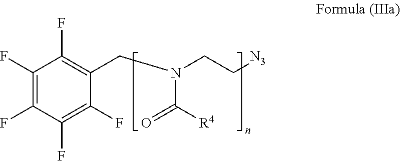
wherein R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
n is integer from 2 to 2000,
is reacted with a nucleophile to obtain a compound of Formula (III) 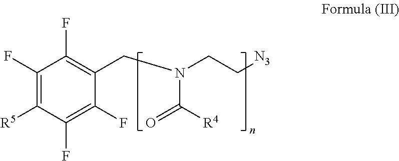
wherein R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R⁵ is selected from the group consisting of halogen, —SR², —OR², —CN, —NHR², —NR² R³, —NHNHR², —N═NR², and —N₃, —NHOR², and —ONR²R³;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted;
R^{C} is selected from the group consisting of hydrogen, alkyl group having 1-20 carbon atoms which may be substituted, or alkenyl group having 2-20 carbon atoms which may be substituted;
said alkyl, and alkenyl are straight chain; and
n is integer from 2 to 2000.

23. The method for producing the compound of formula (III) according to claim 22, wherein the nucleophile is alcohol, phenol, carboxylic acid, amine, azide, thiol, cyan, or alkyne type nucleophile.

24. The method for producing the compound of formula (VI), 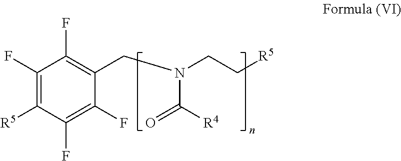
wherein repeating unit is independent of each occurrence and may be identical or different;
R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R⁵ is selected from the group consisting of halogen, —SR², —OR², —CN, —NHR², —NR² R³, —NHNHR², —N═NR², —N₃, —NHOR², and —ONR²R³;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, alkyl group having 1-20 carbon atoms which may be substituted, or alkenyl group having 2-20 carbon atoms which may be substituted;
said alkyl, and alkenyl are straight chain; and
n is integer from 2 to 100,
in which a compound of formula (IIa) 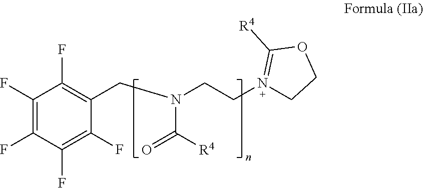
wherein R⁴ is selected from the group consisting of alkyl group having 1-6 carbon atoms which may be substituted, alkenyl group having 2-6 carbon atoms which may be substituted, and alkynyl group having 2-6 carbon atoms which may be substituted;
n is integer from 2 to 2000,
is reacted with nucleophile to obtain the compound of Formula (VI).

25. A method for producing the compound of Formula (Ia) 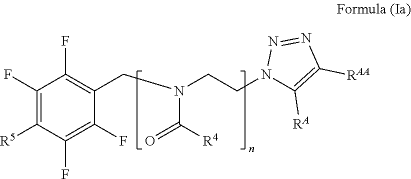
wherein repeating unit is independent of each occurrence and may be identical or different;
R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R⁵ is selected from the group consisting of —SR², —OR², —CN, —NHR², —NR²R³, —NHNHR², —N═NR², —N₃, —NHOR², and —ONR²R³;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted, preferably —CH₂CH(OR^{C})CH₂OR^{C};
R^{A} and R^{AA} are, independent of each other, selected from the group consisting of hydrogen, alkyl group having 1-20 carbon atoms which may be substituted, alkenyl group having 2-20 carbon atoms which may be substituted, and alkynyl group having 2-20 carbon atoms which may be substituted, or R^{A} and R^{AA} may be linked together to form ring, such as carbocycle, heterocycle, aryl, heterocyclyl, lactone, or lactam, and R^{A} and/or R^{AA} may be further linked to functional molecules such as labels, biofunctional molecules including proteins, nucleic acid, etc.;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, lipid soluble groups such as alkyl group having 1-20 carbon atoms which may be substituted, alkenyl group having 2-20 carbon atoms which may be substituted, and alkynyl group having 2-20 carbon atoms which may be substituted, or oxygen protective groups including acyl groups such as acetyl, ether groups such as methoxymethyl etc.;
said alkyl, alkenyl and alkynyl is straight chain, branched chain or cyclic chain; and
n is integer from 1 to 2000,
wherein the compound of Formula (III) 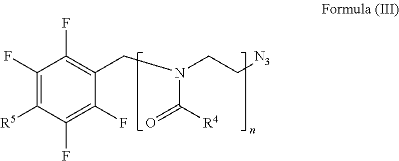
wherein repeating unit is independent of each occurrence and may be identical or different;
R⁴ is selected from the group consisting of alkyl group having 1-6 carbon atoms which may be substituted, alkenyl group having 2-6 carbon atoms which may be substituted, and alkynyl group having 2-6 carbon atoms which may be substituted, aryl which may be substituted, heteroaryl which may be substituted;
R⁵ is selected from the group consisting of halogen, —SR², —OR², —CN, —NHR², —NR² R³, —NHNHR², —N═NR², and —N₃, —NHOR², —ONR²R³;
R² and R³ are, independent of each other, selected from the group consisting of hydrogen, alkyl group having 1-6 carbon atoms which may be substituted, alkenyl group having 2-6 carbon atoms which may be substituted, and alkynyl group having 2-6 carbon atoms which may be substituted, carbocyclyl which may be substituted, heterocyclyl which may be substituted, aryl which may be substituted, heteroaryl which may be substituted, —C(═O)R^{B}, —C(═O)OR^{B}, —C(═O)N(R^{B})₂, —S(═O)R^{B}, —S(═O)₂R^{B}, —CH₂CH(OR^{C})CH₂OR^{C}, and sugar derivatives, or in the case of —NR² R³, R² and R³ may be linked together to form heterocyclyl which may be substituted;
R^{B} is selected from the group consisting of hydrogen, alkyl group having 1-6 carbon atoms which may be substituted, alkenyl group having 2-6 carbon atoms which may be substituted, and alkynyl group having 2-6 carbon atoms which may be substituted;
R^{C} is selected from the group consisting of hydrogen, alkyl group having 1-20 carbon atoms which may be substituted, or alkenyl group having 2-20 carbon atoms which may be substituted;
said alkyl, and alkenyl are straight chain; and
n is integer from 2 to 2000
is reacted with formula (VII) 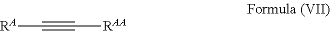
wherein R^{A} and R^{AA} are as shown above,
to obtain the compound of Formula (Ia).

26. The method for producing the compound of formula (Ia) according to claim 25, wherein the reaction carried out with metal catalyst such as Cu catalyst.

27. The method for producing the compound of formula (Ia) according to claim 25, wherein the reaction is carried out under the condition of metal catalyst free.

28. The method for producing the compound of formula (Ia) according to claim 25, wherein the reaction is carried out in water.
